(19) United States
(12) Patent Application Publication
(10) Pub. No.: US 2015/0018404 A1
(43) Pub. Date: Jan. 15, 2015

(54) DOUBLE-STRANDED OLIGONUCLEOTIDE COMPOUNDS FOR TREATING HEARING AND BALANCE DISORDERS

(71) Applicants: Elena Feinstein, Rahovot (IL); Sharon Avkin-Nachum, Nes Ziona (IL); Igor Mett, Rehovot (IL); Hagar Kalinski, Rishon-le-Zion (IL)

(72) Inventors: Elena Feinstein, Rahovot (IL); Sharon Avkin-Nachum, Nes Ziona (IL); Igor Mett, Rehovot (IL); Hagar Kalinski, Rishon-le-Zion (IL)

(73) Assignee: QUARK PHARMACEUTICALS, INC., Fremont, CA (US)

(21) Appl. No.: 14/235,780

(22) PCT Filed: Aug. 3, 2012
(86) PCT No.: PCT/US12/49616

§ 371 Date: Sep. 28, 2014

Related U.S. Application Data
(60) Provisional application No. 61/514,541, filed on Aug. 3, 2011.
(60) Provisional application No. 61/585,672, filed on Jan. 12, 2012.

(51) Int. Cl. C12N 15/113

(52) U.S. Cl. CPC C12N 15/113; C12N 2310/50; C12N 2310/11
USPC 514/44 A; 536/24.5

(57) ABSTRACT

The present application relates to double stranded nucleic acid compounds, compositions comprising same and methods of use thereof for the treatment of hearing loss in a subject in need thereof. The compounds are preferably chemically synthesized and modified dsRNA molecules which inhibit expression of a gene expressed selected from the group consisting of HES1, HES5, HEY1, HEY2, ID1, ID2, ID3, CDKN1B, and NOTCH1.

### RELATED APPLICATIONS

This application claims the benefit of U.S. Provisional Application Ser. No. 61/514,541 filed Aug. 3, 2011 entitled “Compounds, Compositions and Methods For Treating Hearing Loss” and of U.S. Provisional Application Ser. No. 61/585,672 filed Jan. 12, 2012 entitled “Compounds, Compositions and Methods For Treating Hearing Loss” which are incorporated herein by reference in their entirety and for all purposes.

### SEQUENCE LISTING

This application incorporates-by-reference nucleotide and/or amino acid sequences which are present in the file named “120803_{—}2094_{—}84407_PCT_Sequence_Listing_BI”, which is 5.097 megabytes in size, and which was created on Aug. 3, 2012 in the IBM-PC machine format, having an operating system compatibility with MS-Windows, and is submitted herewith.

### FIELD OF THE INVENTION

The present invention relates to compounds, pharmaceutical compositions comprising same and methods of use thereof for the down-regulation of genes associated with hearing loss including HEST, HES5, HEY1, HEY2, ID1, ID2, ID3, CDKN1B, or NOTCH1, the inhibition of which is useful for treating hearing loss, treating balance impairment, promoting the replacement, regeneration, or protection of otic hair (sensory) cells of the inner ear, or effecting hearing restoration/regeneration.

### BACKGROUND OF THE INVENTION

### siRNA and RNA Interference

RNA interference (RNAi) is a phenomenon involving double-stranded (ds) RNA-dependent gene specific posttranscriptional silencing.

### Hearing Disorders/Hearing Loss

Acquired hearing disorders/hearing loss can be caused by several factors including for example, exposure to harmful noise levels, mechanical inner ear trauma, aging processes, exposure to ototoxic drugs such as, without being limited to, cisplatin and aminoglycoside antibiotics and aging.

PCT application publication nos. WO2007084684 and WO 2009/147684 to the assignee of the present application relate to compounds and compositions useful in treating hearing disorders and diseases.

U.S. Pat. No. 7,825,099 to the assignee of the present application relates to use of p53 oligonucleotide inhibitors for treating hearing disorders.

Molecules, compositions, methods and kits useful in treating or attenuating hearing loss treating balance impairment, promoting the replacement, regeneration, or protection of otic (sensory) hair cells of the inner ear, and or effecting hearing restoration/regeneration are needed.

### SUMMARY OF THE INVENTION

Nucleic acid molecules for down-regulating expression of HES1, HES5, HEY1, HEY2, ID1, ID2, ID3, CDKN1B, or NOTCH1, compositions and kits comprising same and methods of use thereof are provided herein. The compositions, methods and kits may involve use of nucleic acid molecules (for example, short interfering nucleic acid (siNA), short interfering RNA (siRNA), double-stranded RNA (dsRNA), micro-RNA (miRNA) or short hairpin RNA (shRNA)) that bind a nucleotide sequence (such as an mRNA sequence) or portion thereof, encoding HES1, HES5, HEY1, HEY2, ID1, ID2, ID3, CDKN1B, or NOTCH1, for example, the mRNA coding sequence (SEQ ID NO:1-11) for human HES1, HES5, HEY1, HEY2, ID1, ID2, ID3 or CDKN1B, encoding one or more proteins or protein subunits exemplified by SEQ ID NO:12-22. In certain preferred embodiments, the molecules, compositions, methods and kits disclosed herein down-regulate or inhibit expression of HES1, HES5, HEY1, HEY2, ID1, ID2, ID3, CDKN1B genes. In various embodiments the nucleic acid molecule is selected from the group consisting of unmodified or chemically modified dsRNA compound such as a siRNA or shRNA that down-regulates HES1, HES5, HEY1, HEY2, ID1, ID2, ID3, CDKN1B, or NOTCH1 expression.

In some preferred embodiments the inhibitor is a synthetic, chemically modified double stranded RNA (dsRNA) compound that down-regulates HES1 expression. In certain preferred embodiments, “HES1” refers to human HES1. In some preferred embodiments the inhibitor is a synthetic, chemically modified double stranded RNA (dsRNA) compound that down-regulates HES5 expression. In certain preferred embodiments, “HES5” refers to human HES5. In some preferred embodiments the inhibitor is a synthetic, chemically modified double stranded RNA (dsRNA) compound that down-regulates HEY1 expression. In certain preferred embodiments, “HEY1” refers to human HEY1. In some preferred embodiments the inhibitor is a synthetic, chemically modified double stranded RNA (dsRNA) compound that down-regulates HEY2 expression. In certain preferred embodiments, “HEY2” refers to human HEY2. In some preferred embodiments the inhibitor is a synthetic, chemically modified double stranded RNA (dsRNA) compound that down-regulates ID1 expression. In certain preferred embodiments, “ID1” refers to human ID1. In some preferred embodiments the inhibitor is a synthetic, chemically modified double stranded RNA (dsRNA) compound that down-regulates ID2 expression. In certain preferred embodiments, “ID2” refers to human ID2. In some preferred embodiments the inhibitor is a synthetic, chemically modified double stranded RNA (dsRNA) compound that down-regulates ID3 expression. In certain preferred embodiments, “ID3” refers to human ID3. In some preferred embodiments the inhibitor is a synthetic, chemically modified double stranded RNA (dsRNA) compound that down-regulates CDKN1B expression. In certain preferred embodiments, “CDKN1B” refers to human CDKN1B. In some preferred embodiments the inhibitor is a synthetic, chemically modified double stranded RNA (dsRNA) compound that down-regulates NOTCH1 expression. In certain preferred embodiments, “NOTCH1” refers to human NOTCH1. In various embodiments a combination of dsRNA to two or more target genes is preferred. In certain preferred embodiments, “target genes” refers to human genes HES1, HES5, HEY1, HEY2, ID1, ID2, ID3, CDKN1B, or NOTCH1. In some embodiments the preferred target genes are selected from the group consisting of HES1, HES5, HEY2, CDKN1B and NOTCH1.

The chemically modified nucleic acid molecules and compositions provided herein exhibit beneficial properties, including at least one of increased serum stability, improved cellular uptake, reduced off-target activity, reduced immunogenicity, improved endosomal release, improved specific delivery to target tissue or cell and increased knock down/down-regulation activity when compared to corresponding unmodified nucleic acid molecules.

Further disclosed herein are methods for treating or preventing the incidence or severity of a disorder, disease, injury or condition in a subject in need thereof wherein the disease or condition and/or a symptom or pathology associated therewith is associated with expression of the HES1, HES5, HEY1, HEY2, ID1, ID2, ID3, CDKN1B, or NOTCH1 gene, such as a disorder, disease, injury, condition or pathology of the inner ear. In some embodiments the subject is a mammal. In a preferred embodiment the subject is a human subject.

In particular embodiments, chemically modified dsRNA compounds that target HES1, HES5, HEY1, HEY2, ID1, ID2, ID3, CDKN1B, or NOTCH1, compositions and kits comprising same and methods of use thereof in the treatment of an ear (otic, aural) condition or pathology, particularly pathologies involving death of otic (sensory) hair cells if the inner ear, are provided herein. Other conditions to be treated include any condition in which HES1, HES5, HEY1, HEY2, ID1, ID2, ID3, CDKN1B, or NOTCH1 expression is detrimental, and are treated with the compounds of the present invention.

In one aspect, provided are nucleic acid molecules (e.g., dsRNA molecules) in which (a) the nucleic acid molecule is a duplex which includes a sense strand and a complementary antisense strand; (b) each strand of the nucleic acid molecule is independently 18 to 49 nucleotides in length; (c) an 18 to 49 nucleotide sequence of the antisense strand is complementary to a consecutive sequence of a mRNA encoding mammalian HES1, HES5, HEY1, HEY2, ID1, ID2, ID3, CDKN1B, or NOTCH1 (e.g., SEQ ID NO: 1-11) or portion thereof; and (d) the sense strand and antisense strand comprise sequence pairs set forth in any of SEQ ID NOS:23-26,666. In some embodiments the sense strand and antisense strand comprise sequence pairs set forth in any of SEQ ID NOS:26,667-26,912.

In another aspect provided are methods for treating, including preventing, the incidence or severity of hearing loss in which expression of one or more of the HES1, HES5, HEY1, HEY2, ID1, ID2, ID3, CDKN1B and NOTCH1 genes is associated with the etiology or progression of the hearing disorder/hearing loss.

In yet another aspect, provided are methods for treating, including preventing, the incidence or severity of balance impairment in which expression of one or more of the HES1, HES5, HEY1, HEY2, ID1, ID2, ID3, CDKN1B and NOTCH1 genes is associated with the etiology or progression of the balance impairment.

In another aspect, provided are methods for treating, including preventing, the incidence or severity of loss of otic (sensory) hair cells of the inner ear, in which expression of one or more of the HES1, HES5, HEY1, HEY2, ID1, ID2, ID3, CDKN1B and NOTCH1 genes is associated with the etiology or progression of the otic (sensory) hair cell loss.

The inhibitory nucleic acids provided herein are preferably dsRNA molecules that possess modifications which may increase activity, increase stability, and/or minimize toxicity when compared to the corresponding unmodified dsRNA compound. These molecules, when admixed with a pharmaceutical vehicle that effects delivery of the nucleic acid to the middle and inner ear, provide effective, safe and patient compliant therapeutic compounds useful in treating a variety of inner ear disorders. The dsRNA molecules are designed to down-regulate target gene expression and attenuate target gene function. In certain embodiment the target gene is transcribed into any one of the mRNA polynucleotides listed in Table 1A, set forth in SE ID NOS:1-11.

The dsRNA molecules provided herein are double-stranded chemically modified oligonucleotides. In some embodiments the sense oligonucleotide and the antisense oligonucleotide useful in generating the chemically modified dsRNA molecules RNAs are selected from sense strand oligonucleotide and corresponding antisense strand oligonucleotide set forth in SEQ ID NOS:23-26912.

In various embodiments of a nucleic acid molecule (e.g., dsRNA molecule) as disclosed herein, the antisense strand may be 18 to 49 nucleotides in length (e.g., 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 nucleotides in length); or 18-35 nucleotides in length; or 18-30 nucleotides in length; or 18-25 nucleotides in length; or 18-23 nucleotides in length; or 19-21 nucleotides in length; or 25-30 nucleotides in length; or 26-28 nucleotides in length. In some embodiments of a nucleic acid molecule (e.g., dsRNA molecule) as disclosed herein, the antisense strand is 19 nucleotides in length. Similarly the sense strand of a nucleic acid molecule (e.g., dsRNA molecule) as disclosed herein may be 18 to 49 nucleotides in length (e.g., 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 nucleotides in length); or 18-35 nucleotides in length; or 18-30 nucleotides in length; or 18-25 nucleotides in length; or 18-23 nucleotides in length; or 19-21 nucleotides in length; or 25-30 nucleotides in length; or 26-28 nucleotides in length. In some embodiments of a nucleic acid molecule (e.g., dsRNA molecule) as disclosed herein, the sense strand is 19 nucleotides in length. In some embodiments of a nucleic acid molecule (e.g., dsRNA molecule) as disclosed herein, each of the antisense strand and the sense strand are 19 nucleotides in length. The duplex region of a nucleic acid molecule (e.g., dsRNA molecule) as disclosed herein may be 18-49 nucleotides in length (e.g., about 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 nucleotides in length), 18-35 nucleotides in length; or 18-30 nucleotides in length; or 18-25 nucleotides in length; or 18-23 nucleotides in length; or 18-21 nucleotides in length; or 25-30 nucleotides in length; or 25-28 nucleotides in length. In various embodiments of a nucleic acid molecule (e.g., dsRNA molecule) as disclosed herein, the duplex region is 19 nucleotides in length.

In certain embodiments, the sense strand and the antisense strand of a nucleic acid (e.g., an dsRNA nucleic acid molecule) as provided herein are separate oligonucleotide strands. In some embodiments, the separate sense strand and antisense strand form a double stranded structure, also known as a duplex, via hydrogen bonding, for example, Watson-Crick base pairing. In some embodiments one or more nucleotide pairs form non-Watson-Crick base pairing. In some embodiments the sense strand and the antisense strand are two separate strands that are covalently linked to each other. In other embodiments, the sense strand and the antisense strands are part of a single oligonucleotide having both a sense and antisense region; in some preferred embodiments the oligonucleotide has a hairpin structure.

In certain embodiments, the nucleic acid molecule is a double stranded nucleic acid (dsRNA) molecule that is symmetrical with regard to overhangs, and has a blunt end on both ends. In other embodiments the nucleic acid molecule is a dsRNA molecule that is symmetrical with regard to overhangs, and has a nucleotide or a non-nucleotide or a combination of a nucleotide and non-nucleotide overhang on both ends of the dsRNA molecule. In certain preferred embodiments, the nucleic acid molecule is a dsRNA molecule that is asymmetrical with regard to overhangs, and has a blunt end on one end of the molecule and an overhang on the other end of the molecule. In some embodiments an asymmetrical dsRNA molecule has a 3′-overhang on one side of a duplex occurring on the sense strand; and a blunt end on the other side of the molecule occurring on both the 5′-end of the sense strand and the 5′-end of the antisense strand. In some embodiments an asymmetrical dsRNA molecule has a 5′-overhang on one side of a duplex occurring on the sense strand; and a blunt end on the other side of the molecule occurring on both the 3′-end of the sense strand and the 3′-end of the antisense strand. In other embodiments an asymmetrical dsRNA molecule has a 3′-overhang on one side of a duplex occurring on the antisense strand; and a blunt end on the other side of the molecule occurring on both the 5′-end of the sense strand and the 5′-end of the antisense strand. In some embodiments an asymmetrical dsRNA molecule has a 5′-overhang on one side of a duplex occurring on the antisense strand; and a blunt end on the other side of the molecule occurring on both the 3′-end of the sense strand and the 3′-end of the antisense strand. In some embodiments the overhangs are nucleotide overhangs, in other embodiments the overhangs are non-nucleotide overhangs. In some embodiments the overhangs are 5′ overhangs; in alternative embodiments the overhangs are 3′ overhangs.

In some embodiments, the nucleic acid molecule has a hairpin structure (having the sense strand and antisense strand on one oligonucleotide), with a loop structure on one end and a blunt end on the other end. In some embodiments, the nucleic acid molecule has a hairpin structure, with a loop structure on one end and an overhang end on the other end; in certain embodiments, the overhang is a 3′-overhang; in certain embodiments the overhang is a 5′-overhang; in certain embodiments the overhang is on the sense strand; in certain embodiments the overhang is on the antisense strand.

The nucleic acid molecule (e.g., dsRNA molecule) disclosed herein may include one or more modifications or modified nucleotides such as described herein. For example, a nucleic acid molecule (e.g., dsRNA molecule) as provided herein may include a modified nucleotide having a modified sugar; a modified nucleotide having a modified nucleobase; or a modified nucleotide having a modified phosphate group. Similarly, a nucleic acid molecule (e.g., dsRNA molecule) as provided herein may include a modified phosphodiester backbone and/or may include a modified terminal phosphate group.

A nucleic acid molecule (e.g., dsRNA molecules) as provided herein may have one or more ribonucleotides that include a modified sugar moiety, for example as described herein. A non-limiting example of a modified sugar moiety is a 2′ alkoxy modified sugar moiety. In some preferred embodiments the nucleic acid comprises at least one 2′-O-methyl sugar modified ribonucleotide.

A nucleic acid molecule (e.g., dsRNA molecule) as provided herein may have one or more modified nucleobase(s), for example as described herein.

A nucleic acid molecule (e.g., dsRNA molecule) as provided herein may have one or more modifications to the phosphodiester backbone, for example as described herein.

A nucleic acid molecule (e.g., dsRNA molecule) as provided herein may have one or more modified phosphate group(s), for example as described herein.

In various embodiments, the provided nucleic acid molecule (e.g., dsRNA molecule) may include an unmodified antisense strand and a sense strand having one or more modifications. In some embodiments the provided nucleic acid molecule (e.g., dsRNA molecule) may include an unmodified sense strand and an antisense strand having one or more modifications. In preferred embodiments the provided nucleic acid molecule (e.g., dsRNA molecule) may include one or more modified nucleotides in the both the sense strand and the antisense strand.

A nucleic acid molecule (e.g., dsRNA molecules) as provided herein may include a phosphate group at the 5′ end of the sense and/or the antisense strand (i.e. a 5′-terminal phosphate group). In some embodiments a dsRNA molecule disclosed herein may include a phosphate group at the 5′ terminus of the antisense strand.

A nucleic acid molecule (e.g., dsRNA molecules) as provided herein may include a phosphate group at the 3′ end of the sense and/or the antisense strand (i.e. a 3′-terminal phosphate group). In some embodiments a dsRNA molecule disclosed herein may include a phosphate group at the 3′ terminus of the antisense strand.

In some embodiments a nucleic acid molecule (e.g., dsRNA molecules) disclosed herein may include a phosphate group at the 3′ terminus of the antisense strand and the sense strand.

In some embodiments a nucleic acid molecule (e.g., dsRNA molecules) disclosed herein the antisense strand and the sense strand of the nucleic acid molecule are non-phosphorylated at both the 3′ terminus and at the 5′ terminus.

In some embodiments provided are double stranded nucleic acid compounds useful for down-regulating expression of HES1, HES5, HEY1, HEY2, ID1, ID2, ID3. CDKN1B, or NOTCH1 gene. In some embodiments provided herein is a double stranded RNA (dsRNA) molecule having the structure (A1):

| | | |
|---|---|---|
| (A1) | 5′ (N)x-Z 3′ | (antisense strand) |
| | 3′ Z′-(N′)y-z″ 5′ | (sense strand) |

wherein each N and N′ is a ribonucleotide which may be unmodified or modified, or an unconventional moiety; wherein each of (N)x and (N′)y is an oligonucleotide in which each consecutive N or N′ is joined to the next N or N′ by a covalent bond;
wherein each of Z and Z′ is independently present or absent, but if present independently comprises 1-5 consecutive nucleotides, 1-5 consecutive non-nucleotide moieties or a combination thereof covalently attached at the 3′ terminus of the strand in which it is present;
wherein z″ may be present or absent, but if present is a capping moiety covalently attached at the 5′ terminus of (N′)y;
each of x and y is independently an integer from 18 to 40;
wherein the sequence of (N′)y is complementary to the sequence of (N)x; and wherein (N)x comprises an antisense sequence and (N′)y comprises a sense sequence set forth in any one of SEQ ID NOS:23-693 and 26691-26706 (HES1); SEQ ID NOS:1496-2029 and 26725-26732 (HES5); SEQ ID NOS:2704-3025 and 26809-26816 (ID1); SEQ ID NOS:3634-5053 and 26825-26832 (ID2); SEQ ID NOS:6206-6671 and 26851-26866 (ID3); SEQ ID NOS:7444-9007 and 26887-26900 (CDKN1B); SEQ ID NOS:10534-11549 and 26761-26778 (HEY1); SEQ ID NOS:13004-14801 and 26785-26788 (HEY2); SEQ ID NOS:16622-18643 and 26922-26912 (NOTCH1).

In some embodiments preferred (N)x and (N′)y are set forth in any one of SEQ ID NOS:26691-26706 (HES1); SEQ ID NOS:26725-26732 (HES5); SEQ ID NOS: 26809-26816 (ID1); SEQ ID NOS:26825-26832 (ID2); SEQ ID NOS:26851-26866 (ID3); SEQ ID NOS:26887-26900 (CDKN1B); SEQ ID NOS:26761-26778 (HEY1); SEQ ID NOS:26785-26788 (HEY2); SEQ ID NOS:26922-26912 (NOTCH1).

In some embodiments the covalent bond joining each consecutive N and/or N′ is a phosphodiester bond.

In some embodiments x=y and each of x and y is 19, 20, 21, 22 or 23. In preferred embodiments x=y=19.

In some embodiments the double-stranded nucleic acid molecule comprises an (N)x and (N′)y selected from the nucleic acid described as HES5_{—}8 (SEQ ID NOS:26732 and 26728). In some embodiments (N)x comprises at least one 2′OMe sugar modified pyrimidine or purine ribonucleotide; optionally a 2′5′ ribonucleotide in position 7 and a nucleotide or non-nucleotide moiety (Z) covalently attached to the 3′ terminus; and wherein (N′)y comprises at least one 2′OMe sugar modified ribonucleotide and/or 3-5 consecutive 2′5′ ribonucleotides in the 3′ terminal positions (5′>3′); z′ is present and wherein a nucleotide or non-nucleotide moiety (Z″) is covalently attached to the 3′ terminus. In some embodiments in (N)x 1-8 pyrimidines are 2′OMe sugar modified ribonucleotides, a 2′5′ ribonucleotide is present is in position 7; and Z is present; and wherein (N′)y comprises five 2′-5′ ribonucleotides in positions 15-19; wherein z″ is present and wherein Z′ is present. In preferred embodiments z″ comprises an inverted abasic moiety; wherein Z′ comprises a C3Pi moiety; and wherein Z comprises a C3Pi-C3OH moiety.

In some embodiments the double-stranded nucleic acid molecule comprises an (N)x and (N′)y selected from the nucleic acid described as CDKN1B_{—}4 (SEQ ID NOS:26894 and 26887). In some embodiments (N)x comprises at least one 2′OMe sugar modified pyrimidine or purine ribonucleotide; optionally a 2′5′ ribonucleotide in position 7 and a nucleotide or non-nucleotide moiety (Z) covalently attached to the 3′ terminus; and wherein (N′)y comprises at least one 2′OMe sugar modified ribonucleotide and/or 3-5 consecutive 2′5′ ribonucleotides in the 3′ terminal positions (5′>3′); z′ is present and wherein a nucleotide or non-nucleotide moiety (Z″) is covalently attached to the 3′ terminus. In some embodiments in (N)x 2′OMe sugar modified ribonucleotides are present in positions 1, 13 and 17, optionally a 2′5′ ribonucleotide is present is in position 7; and Z is present; and wherein (N′)y comprises 2′OMe sugar modified ribonucleotides in positions 2, 11, 15, and 18, wherein z″ is present and wherein Z′ is present. In preferred embodiments z″ comprises an inverted abasic moiety or an amino C3 moiety; wherein Z′ comprises a C3Pi moiety; and wherein Z comprises a C3Pi-C3OH moiety.

In some embodiments of nucleic acid molecules (e.g., dsRNA molecules) as disclosed in structure (A1) herein, the double stranded nucleic acid molecule is a siRNA, siNA or a miRNA. In some embodiments of nucleic acid molecules (e.g., dsRNA molecules) of Structure (A1) as disclosed herein, the double stranded nucleic acid molecule is a chemically modified siRNA.

In some embodiments the double stranded nucleic acid molecules comprise a DNA moiety or a mismatch to the target at position 1 of the antisense strand (5′ terminus). Such a duplex structure is described herein. According to one embodiment provided herein is a double stranded dsRNA molecule having a structure (A2) set forth below:

| | | |
|---|---|---|
| (A2) | 5′ N1-(N)x-Z 3′ | (antisense strand) |
| | 3′ Z′-N2-(N′)y-z″ 5′ | (sense strand) |

wherein each N1, N2, N and N′ is independently an unmodified or modified nucleotide, or an unconventional moiety;
wherein each of (N)x and (N′)y is an oligonucleotide in which each consecutive N or N′ is joined to the adjacent N or N′ by a covalent bond;
wherein each of x and y is independently an integer between 17 and 39;
wherein N2 is covalently bound to (N′)y;
wherein N1 is covalently bound to (N)x and is mismatched to the target mRNA (SEQ ID NO:1-11) or is a complementary DNA moiety to the target mRNA;
wherein N1 is a moiety selected from the group consisting of natural or modified: uridine, deoxyribouridine, ribothymidine, deoxyribothymidine, adenosine or deoxyadenosine, an abasic ribose moiety and an abasic deoxyribose moiety;
wherein z″ may be present or absent, but if present is a capping moiety covalently attached at the 5′ terminus of N2-(N′)y;
wherein each of Z and Z′ is independently present or absent, but if present is independently 1-5 consecutive nucleotides, 1-5 consecutive non-nucleotide moieties or a combination thereof covalently attached at the 3′ terminus of the strand in which it is present; and
wherein the sequence of (N′)y is complementary to the sequence of (N)x; and wherein the sequence of (N)x comprises an antisense sequence and (N′)y comprises a sense sequence set forth in any one of SEQ ID NOS:694-1495 and 26667-26690 (HES1); SEQ ID NOS:2030-2703 and 26707-26724 (HES5); SEQ ID NOS:3026-3633 and 26789-26808 (ID1); SEQ ID NOS:5054-6205 and 26817-26824 (ID2); SEQ ID NOS:6672-7443 and 26833-26850 (ID3); SEQ ID NOS:9008-10533 and 26867-26886 (CDKN1B); SEQ ID NOS:11550-13003 and 26733-26760 (HEY1); SEQ ID NOS:14802-16389 and 26779-26784 (HEY2); SEQ ID NOS:18644-26666 and 2601-26910 (NOTCH1). Preferred (N)x and (N′)y are set forth in any one of SEQ ID NOS:26667-26690 (HES1); SEQ ID NOS:26707-26724 (HES5); SEQ ID NOS:26789-26808 (ID1); SEQ ID NOS:26817-26824 (ID2); SEQ ID NOS: 26833-26850 (ID3); SEQ ID NOS:26867-26886 (CDKN1B); SEQ ID NOS:26733-26760 (HEY1); SEQ ID NOS:26779-26784 (HEY2); SEQ ID NOS:2601-26910 (NOTCH1). Molecules covered by the description of Structure (A2) are also referred to herein as “18+1” or “18+1 mer”.

In some embodiments the N2-(N′)y and N1-(N)x useful in generating dsRNA compounds are presented in Tables I-IX, particularly the sequences designated as “18+1” type.

In certain embodiments of Structure (A2), (N)x of a nucleic acid molecule (e.g., a dsRNA molecule) as disclosed herein includes a sequence corresponding to any one of the antisense sequences SEQ ID NO:23-26912. In certain preferred embodiments (N)x and (N′)y are selected from the sequence pairs shown in Tables I-IX (SEQ ID NOS:26667-26912).

In some embodiments of Structure (A2), the sequence of (N′)y is fully complementary to the sequence of (N)x. In various embodiments sequence of N2-(N′)y is complementary to the sequence of N1-(N)x. In some embodiments (N)x comprises an antisense that is fully complementary to about 17 to about 39 consecutive nucleotides in a target mRNA set forth in SEQ ID NO:1-11. In other embodiments (N)x comprises an antisense that is substantially complementary to about 17 to about 39 consecutive nucleotides in a target mRNA set forth in SEQ ID NO:1-11.

In some embodiments of Structure (A2), N1 and N2 form a Watson-Crick base pair. In other embodiments N1 and N2 form a non-Watson-Crick base pair. In some embodiments a base pair is formed between a ribonucleotide and a deoxyribonucleotide.

In some embodiments of Structure (A2), x=y=18, x=y=19 or x=y=20. In preferred embodiments x=y=18. When x=18 in N1-(N)x, N1 refers to position 1 and positions 2-19 are included in (N)18. When y=18 in N2-(N′)y, N2 refers to position 19 and positions 1-18 are included in (N′)18.

In some embodiments of Structure (A2), N1 is covalently bound to (N)x and is mismatched to the target mRNA set forth in SEQ ID NO:1-11. In various embodiments N1 is covalently bound to (N)x and is a DNA moiety complementary to the target mRNA set forth in SEQ ID NO:1-11.

In some embodiments of Structure (A2), a uridine in position 1 of the antisense strand is substituted with an N1 selected from natural or modified: adenosine, deoxyadenosine, uridine, deoxyuridine (dU), ribothymidine or deoxythymidine. In various embodiments N1 is selected from natural or modified: adenosine, deoxyadenosine or deoxyuridine. For example, in some embodiments a cytidine in position 1 is replaced with an adenine or a uridine; a guanosine in position 1 is replaced with an adenine or a uridine; or an adenine is replaced with a uridine.

In some embodiments of Structure (A2), guanosine in position 1 (N1) of the antisense strand is substituted with a natural or modified: adenosine, deoxyadenosine, uridine, deoxyuridine, ribothymidine or deoxythymidine. In various embodiments N1 is selected from a natural or modified: adenosine, deoxyadenosine, uridine or deoxyuridine.

In some embodiments of Structure (A2), cytidine in position 1 (N1) of the antisense strand is substituted with a natural or modified: adenosine, deoxyadenosine, uridine, deoxyuridine, ribothymidine or deoxythymidine. In various embodiments N1 is selected from a natural or modified: adenosine, deoxyadenosine, uridine or deoxyuridine.

In some embodiments of Structure (A2), adenosine in position 1 (N1) of the antisense strand is substituted with a natural or modified: deoxyadenosine, deoxyuridine, ribothymidine or deoxythymidine.

In some embodiments of Structure (A2), N1 and N2 form a base pair between natural or modified: uridine or deoxyuridine, and adenosine or deoxyadenosine. In other embodiments N1 and N2 form a base pair between natural or modified: deoxyuridine and adenosine.

In some embodiments of Structure (A2), the double stranded nucleic acid molecule is a siRNA, siNA or a miRNA. The double stranded nucleic acid molecules as provided herein are also referred to as “duplexes”. In some embodiments of nucleic acid molecules (e.g., dsRNA molecules) according to Structure (A2) as disclosed herein, the double stranded nucleic acid molecule is a chemically modified siRNA.

In certain preferred embodiments of Structure (A2), x=y=18. In some embodiments x=y=18 and (N)x consists of an antisense oligonucleotide present in SEQ ID NOS:694-1495 and 26667-26690 (HEST); SEQ ID NOS:2030-2703 and 26707-26724 (HES5); SEQ ID NOS:3026-3633 and 26789-26808 (ID1); SEQ ID NOS:5054-6205 and 26817-26824 (ID2); SEQ ID NOS:6672-7443 and 26833-26850 (ID3); SEQ ID NOS:9008-10533 and 26867-26886 (CDKN1B); SEQ ID NOS:11550-13003 and 26733-26760 (HEY1); SEQ ID NOS:14802-16389 and 26779-26784 (HEY2); SEQ ID NOS:18644-26666 and 2601-26910 (NOTCH1).

In some embodiments, N1 is selected from a natural uridine and a modified uridine. In some embodiments, N1 is a natural uridine. In some embodiments, (N)x comprises an antisense oligonucleotide and (N′)y comprises a sense oligonucleotide present in sequence pairs set forth in SEQ ID NOS:694-1495 (HES1); SEQ ID NOS:2030-2703 (HES5); SEQ ID NOS:3026-3633 (ID1); SEQ ID NOS:5054-6205 (ID2); SEQ ID NOS:6672-7443 (ID3); SEQ ID NOS:9008-10533 (CDKN1B); SEQ ID NOS:11550-13003 (HEY1); SEQ ID NOS:14802-16389 (HEY2); SEQ ID NOS:18644-26666 (NOTCH1).

In some embodiments x=y=18 and N1-(N)x comprises an antisense oligonucleotide and N2-(N′)y comprises a sense oligonucleotide present in sequence pairs set forth in SEQ ID 26667-26690 (HES1); SEQ ID NOS: 26707-26724 (HES5); SEQ ID NOS: 26789-26808 (ID1); SEQ ID NOS: 26817-26824 (ID2); SEQ ID NOS: 26833-26850 (ID3); SEQ ID NOS: 26867-26886 (CDKN1B); SEQ ID NOS: 26733-26760 (HEY1); SEQ ID NOS: 26779-26784 (HEY2); SEQ ID NOS: 2601-26910 (NOTCH1).

In some embodiments, x=y=18 and N1 is selected from a natural or modified uridine, a natural or modified adenine, and a natural or modified thymidine.

In some embodiments of Structure (A2), N1 is a 2′OMe sugar-modified uridine or a 2′OMe sugar-modified adenosine. In certain embodiments of structure (A2), N2 is a 2′OMe sugar modified ribonucleotide or deoxyribonucleotide.

In some embodiments the double-stranded nucleic acid molecule comprises an (N)x and (N′)y selected from the nucleic acid described as HES1_{—}36 (SEQ ID NOS:26690 and 26678). In some embodiments (N)x comprises 2′OMe sugar modified ribonucleotides, and optionally a 2′-5′ ribonucleotide in at least one of positions 5, 6, or 7; wherein (N′)y comprises at least one 2′5′ ribonucleotide or 2′OMe modified ribonucleotide; wherein z″ is present; and wherein each of Z and Z′ is present and consists of a non-nucleotide moiety covalently attached to the 3′ terminus of the strand in which it is present. In preferred embodiments (N)x comprises 2′OMe sugar modified ribonucleotides at positions 3, 9, 11 and 15; and wherein (N′)y comprises five 2′5′ ribonucleotides at the 3′ terminal positions 15, 16, 17, 18, and 19. In preferred embodiments z″ comprises an inverted abasic moiety; wherein Z′ comprises a C3Pi moiety; and wherein Z comprises a C3Pi-C3OH moiety.

In some embodiments the double-stranded nucleic acid molecule comprises an (N)x and (N′)y selected from the nucleic acid described as HES1_{—}14 (SEQ ID NOS:26681 and 26669). In some embodiments (N)x comprises 2′OMe sugar modified ribonucleotides, and optionally a 2′-5′ ribonucleotide in at least one of positions 5, 6, or 7; wherein (N′)y comprises at least one 2′5′ ribonucleotide or 2′OMe modified ribonucleotide; wherein z″ is present; and wherein each of Z and Z′ is present and consists of a non-nucleotide moiety covalently attached to the 3′ terminus of the strand in which it is present. In some embodiments (N)x comprises 2′OMe sugar modified ribonucleotides at positions 1, 3, 9, 11, 15 and 18 (5′>3′); and wherein (N′)y comprises a 2′OMe sugar modified ribonucleotide at position 1, and 5 consecutive 2′5′ ribonucleotides in the 3′ terminal positions 15, 16, 17, 18, and 19 (5′>3′). In some embodiments (N)x comprises 2′OMe sugar modified ribonucleotides at positions 1, 3, 9, 11, 14, 15 and 18 (5′>3′), and a 2′5′ ribonucleotide in position 7; and wherein (N′)y comprises a 2′OMe sugar modified ribonucleotide at position 1, and 5 consecutive 2′5′ ribonucleotides in the 3′ terminal positions 15, 16, 17, 18, and 19 (5′>3′). In some embodiments (N)x comprises 2′OMe sugar modified ribonucleotides at positions 1, 3, 9, 11, 14, 15 and 18 (5′>3′), a 2′5′ ribonucleotide present in position 7, and wherein Z is present; and wherein (N′)y comprises 2′OMe sugar modified ribonucleotides at positions 1, 4, 8, 10, 12 and 16 and wherein z″ and Z′ are present. In preferred embodiments z″ comprises an inverted abasic moiety; wherein Z′ comprises a C3Pi moiety; and wherein Z comprises a C3Pi-C3OH moiety.

In some embodiments the double-stranded nucleic acid molecule comprises an (N)x and (N′)y selected from the nucleic acid described as HEY2_{—}1 (SEQ ID NOS:26747 and 26733). In some embodiments (N)x comprises at least one 2′OMe sugar modified pyrimidine ribonucleotide; optionally a 2′5′ ribonucleotide in position 7 and a nucleotide or non-nucleotide moiety covalently attached to the 3′ terminus; and wherein (N′)y comprises at least one 2′OMe sugar modified ribonucleotides and/or 3-5 consecutive 2′5′ ribonucleotides in the 3′ terminal positions (5′>3′); z′ is present and wherein a nucleotide or non-nucleotide moiety is covalently attached to the 3′ terminus. In some embodiments in (N)x 1-8 of the pyrimidine ribonucleotides are 2′OMe sugar modified ribonucleotides, a 2′5′ ribonucleotide is present is in position 7; and Z is present; and wherein (N′)y comprises 1-8 of the pyrimidine ribonucleotides are 2′OMe sugar modified ribonucleotides; wherein z″ is present and wherein Z′ is present. In preferred embodiments z″ comprises an inverted abasic moiety; wherein Z′ comprises a C3Pi moiety; and wherein Z comprises a C3Pi-C3OH moiety.

In some embodiments the double-stranded nucleic acid molecule comprises an (N)x and (N′)y selected from the nucleic acid described as HEY2_{—}2 (SEQ ID NOS:26748 and 26734). In some embodiments the double-stranded nucleic acid molecule comprises an (N)x and (N′)y selected from the nucleic acid described as HEY2_{—}1 (SEQ ID NOS:26747 and 26733). In some embodiments (N)x comprises at least one 2′OMe sugar modified pyrimidine ribonucleotide; optionally a 2′5′ ribonucleotide in position 7 and a nucleotide or non-nucleotide moiety covalently attached to the 3′ terminus; and wherein (N′)y comprises at least one 2′OMe sugar modified ribonucleotides and/or 3-5 consecutive 2′5′ ribonucleotides in the 3′ terminal positions (5′>3′); z′ is present and wherein a nucleotide or non-nucleotide moiety is covalently attached to the 3′ terminus. In some embodiments in (N)x 1-8 of the pyrimidine ribonucleotides are 2′OMe sugar modified ribonucleotides, a 2′5′ ribonucleotide is present is in position 7; and Z is present; and wherein (N′)y comprises 1-8 of the pyrimidine ribonucleotides are 2′OMe sugar modified ribonucleotides; wherein z″ is present and wherein Z′ is present. In preferred embodiments z″ comprises an inverted abasic moiety; wherein Z′ comprises a C3Pi moiety; and wherein Z comprises a C3Pi-C3OH moiety.

In some embodiments the double-stranded nucleic acid molecule comprises an (N)x and (N′)y selected from the nucleic acid described as CDKN1B_{—}31 (SEQ ID NOS:26879 and 26869). In some embodiments the double-stranded nucleic acid molecule comprises an (N)x and (N′)y selected from the nucleic acid described as HEY2_{—}1 (SEQ ID NOS:26747 and 26733). In some embodiments (N)x comprises at least one 2′OMe sugar modified pyrimidine ribonucleotide; optionally a 2′5′ ribonucleotide in position 7 and a nucleotide or non-nucleotide moiety covalently attached to the 3′ terminus; and wherein (N′)y comprises at least one 2′OMe sugar modified ribonucleotides and/or 3-5 consecutive 2′5′ ribonucleotides in the 3′ terminal positions (5′>3′); z′ is present and wherein a nucleotide or non-nucleotide moiety is covalently attached to the 3′ terminus. In some embodiments in (N)x 2′OMe sugar modified ribonucleotides are present in positions 1, 13 and 17 or in positions 1, 9, 13, 15 and 18, optionally a 2′5′ ribonucleotide is present is in position 7; and Z is present; and wherein (N′)y comprises 2′OMe sugar modified ribonucleotides in positions 2, 6, 11, 12, 13, 16 and 18; wherein z″ is present and wherein Z′ is present. In some embodiments in (N)x 2′OMe sugar modified ribonucleotides are present in positions 1, 13 and 17 or in positions 1, 9, 13, 15 and 18, optionally a 2′5′ ribonucleotide is present is in position 7; and Z is present; and wherein (N′)y comprises 2′ 5′ ribonucleotides in positions 15-19; wherein z″ is present and wherein Z′ is present. In preferred embodiments z″ comprises an inverted abasic moiety; wherein Z′ comprises a C3Pi moiety; and wherein Z comprises a C3Pi-C3OH moiety.

In some embodiments the double-stranded nucleic acid molecule comprises an (N)x and (N′)y selected from the nucleic acid described as NOTCH1_{—}2 (SEQ ID NOS:26907 and 26902). In some embodiments (N)x comprises at least one 2′OMe sugar modified pyrimidine or purine ribonucleotide; optionally a 2′5′ ribonucleotide in position 7 and a nucleotide or non-nucleotide moiety (Z) covalently attached to the 3′ terminus; and wherein (N′)y comprises at least one 2′OMe sugar modified ribonucleotide and/or 3-5 consecutive 2′5′ ribonucleotides in the 3′ terminal positions (5′>3′); z′ is present and wherein a nucleotide or non-nucleotide moiety (Z″) is covalently attached to the 3′ terminus. In some embodiments in (N)x 2′OMe sugar modified ribonucleotides are present in positions 1, 13 and 17 or in positions 1, 3, 5, 9, 11, and 17, optionally a 2′5′ ribonucleotide is present is in position 7; and Z is present; and wherein (N′)y comprises 2′OMe sugar modified ribonucleotides in positions 15 and 17, wherein z″ is present and wherein Z′ is present. In some embodiments in (N)x 2′OMe sugar modified ribonucleotides are present in positions 1, 13 and 17 or in positions 1, 3, 5, 9, 11, and 17, optionally a 2′5′ ribonucleotide is present is in position 7; and Z is present; and wherein (N′)y comprises five 2′-5′ ribonucleotides in positions 15-19; wherein z″ is present and wherein Z′ is present. In preferred embodiments z″ comprises an inverted abasic moiety; wherein Z′ comprises a C3Pi moiety; and wherein Z comprises a C3Pi-C3OH moiety.

In some embodiments of Structure (A1) and/or Structure (A2), each N consists of an unmodified ribonucleotide. In some embodiments of Structure (A1) and/or Structure (A2) each N′ consists of an unmodified ribonucleotide. In preferred embodiments at least one of N and/or N′ comprises a chemically modified ribonucleotide, an unmodified deoxyribonucleotide, a chemically modified deoxyribonucleotide or an unconventional moiety. In some embodiments the unconventional moiety is selected from a mirror nucleotide, an abasic ribose moiety and an abasic deoxyribose moiety. In some embodiments the unconventional moiety is a mirror nucleotide, preferably an L-DNA moiety. In some embodiments at least one of N or N′ comprises a 2′OMe sugar-modified ribonucleotide.

In some embodiments of Structure (A1) and/or Structure (A2) the sequence of (N′)y is fully complementary to the sequence of (N)x. In other embodiments of Structure (A1) and/or Structure (A2) the sequence of (N′)y is substantially complementary to the sequence of (N)x.

In some embodiments of Structure (A1) and/or Structure (A2) (N)x includes an antisense sequence that is fully complementary to about 17 to about 39 consecutive nucleotides in a target mRNA set forth in any one of SEQ ID NO:1-11. In other embodiments of Structure

A1 and/or Structure A2 (N)x includes an antisense that is substantially complementary to about 17 to about 39 consecutive nucleotides in a target mRNA set forth in any one of SEQ ID NO:1-11. In some embodiments of Structure (A1) and/or Structure (A2), the dsRNA compound is blunt ended, for example, wherein each of z″, Z and Z′ is absent. In an alternative embodiment, at least one of z″, Z or Z′ is present.

In various embodiments Z and Z′ independently include one or more covalently linked modified and or unmodified nucleotides, including deoxyribonucleotides and ribonucleotides, or one or more unconventional moieties for example inverted abasic deoxyribose moiety or abasic ribose moiety or a mirror nucleotide; one or more non-nucleotide C3 moiety or a derivative thereof, non-nucleotide C4 moiety or a derivative thereof or non-nucleotide C5 moiety or a derivative thereof, an non-nucleotide amino-C6 moiety or a derivative thereof, as defined herein, and the like. In some embodiments Z′ is absent and Z is present and includes one or more non-nucleotide C3 moieties. In some embodiments Z is absent and Z′ is present and includes one or more non-nucleotide C3 moieties. In some embodiments each of Z and Z′ independently comprises one or more non-nucleotide C3 moieties or one or more non-nucleotide amino-C6 moieties. In some embodiments z″ is present and is selected from a mirror nucleotide, an abasic moiety and an inverted abasic moiety. In some embodiments of Structures (A1) and/or (A2) each of Z and Z′ includes an abasic moiety, for example a deoxyriboabasic moiety (referred to herein as “dAb”) or riboabasic moiety (referred to herein as “rAb”). In some embodiments each of Z and/or Z′ comprises two covalently linked abasic moieties and is for example dAb-dAb or rAb-rAb or dAb-rAb or rAb-dAb, wherein each moiety is covalently attached to an adjacent moiety, preferably via a phospho-based bond. In some embodiments the phospho-based bond includes a phosphorothioate, a phosphonoacetate or a phosphodiester bond. In preferred embodiments the phospho-based bond is a phosphodiester bond.

In some embodiments each of Z and/or Z′ independently includes an alkyl moiety, optionally propane [(CH2)₃] moiety (C3) or a derivative thereof including propanol (C3OH) and phospho derivative of propanediol (“C3Pi”). In some embodiments each of Z and/or Z′ includes two alkyl moieties and in some examples is C3Pi-C3OH. In the example of C3Pi-C3OH, the 3′ terminus of the antisense strand and/or the 3′ terminus of the sense strand is covalently attached to a C3 moiety via a phospho-based bond and the C3 moiety is covalently bound to a C3OH moiety via a phospho-based bond. In some embodiments the phospho-based bonds include a phosphorothioate, a phosphonoacetate or a phosphodiester bond. In preferred embodiments the phospho-based bond is a phosphodiester bond.

In specific embodiments of Structures (A1) and (A2), Z comprises C3Pi-C3OH. In specific embodiments of Structures (A1) and (A2), Z′ comprises C3Pi or C3OH. In some embodiments of Structures (A1) and (A2), a double stranded nucleic acid molecule includes a C3Pi-C3OH moiety covalently attached to the 3′ terminus of the antisense strand and a C3Pi or C3OH moiety covalently attached to the 3′ terminus of the sense strand.

In some embodiments of Structure (A1) and/or Structure (A2) each N consists of an unmodified ribonucleotide. In some embodiments of Structure (A1) and/or Structure (A2) each N′ consists of an unmodified ribonucleotide. In preferred embodiments, at least one of N and/or N′ is a chemically modified ribonucleotide, an unmodified deoxyribonucleotide, a chemically modified deoxyribonucleotide or an unconventional moiety.

In other embodiments a compound of Structure (A1) and/or (A2) includes at least one ribonucleotide modified in its sugar residue. In some embodiments the compound comprises a modification at the 2′ position of the sugar residue. In some embodiments the modification in the 2′ position comprises the presence of an amino, a fluoro, an alkoxy or an alkyl moiety. In certain embodiments the 2′ modification includes an alkoxy moiety. In preferred embodiments the alkoxy moiety is a methoxy moiety (also referred to as 2′-O-methyl; 2′OMe; 2′OMe; 2′-OCH₃). In some embodiments a nucleic acid compound includes 2′OMe sugar modified alternating ribonucleotides in one or both of the antisense strand and the sense strand. In other embodiments a compound includes 2′OMe sugar modified ribonucleotides in the antisense strand, (N)x or N¹—(N)x, only. In some embodiments, the 2′OMe sugar modified ribonucleotides alternate with unmodified nucleotides. In certain embodiments the middle ribonucleotide of the antisense strand; e.g. ribonucleotide in position 10 in a 19-mer strand, is unmodified. In various embodiments the nucleic acid compound includes at least 5 alternating 2′OMe sugar modified ribonucleotides and unmodified ribonucleotides. In additional embodiments a compound of Structure (A1) and/or (A2) includes modified ribonucleotides in alternating positions wherein each ribonucleotide at the 5′ terminus and at the 3′ terminus of (N)x or N¹—(N)x is modified in its sugar residue, and each ribonucleotide at the 5′ terminus and at the 3′ terminus of (N′)y or N²—(N)y is unmodified in its sugar residue. In various embodiments the ribonucleotides in alternating positions are modified at the 2′ position of the sugar residue.

In some embodiments the nucleic acid compound includes at least 5 alternating 2′OMe sugar modified ribonucleotides and unmodified ribonucleotides, for example at positions 1, 3, 5, 7 and 9 or at positions 11, 13, 15, 17, 19 (5′>3′). In some embodiments, (N)x of Structure (A1) or N1-(N)x of Structure (A2) includes 2′OMe sugar modified ribonucleotides in positions 2, 4, 6, 8, 11, 13, 15, 17 and 19. In some embodiments, (N)x of Structure (A1) or N1-(N)x of Structure (A2) includes 2′OMe sugar modified ribonucleotides in positions 1, 3, 5, 7, 9, 11, 13, 15, 17 and 19. In some embodiments, (N)x of Structure (A1) or N1-(N)x of Structure (A2) includes 2′OMe sugar modified ribonucleotides in one or more pyrimidines.

In some embodiments of Structure (A1) and/or (A2), neither of the sense strand nor the antisense strand is phosphorylated at the 3′ terminus and at the 5′ terminus. In other embodiments one or both of the sense strand and/or the antisense strand are phosphorylated at the 3′ termini. In other embodiments one or both of the sense strand and/or the antisense strand are phosphorylated at the 5′ terminus.

In some embodiments the double stranded molecule disclosed herein includes one or more of the following modifications:
N in at least one of positions 5, 6, 7, 8, or 9 from the 5′ terminus of the antisense strand is selected from a DNA, TNA, a 2′5′ nucleotide or a mirror nucleotide;
N′ in at least one of positions 9 or 10 from the 5′ terminus of the sense strand is selected from a TNA, 2′5′ nucleotide and a pseudoUridine;
N′ in 4, 5, or 6 consecutive positions at the 3′ terminus of (N′)y comprises a 2′5′ ribonucleotide;
one or more pyrimidine ribonucleotides are 2′ sugar modified in the sense strand, the antisense strand or both the sense strand and the antisense strand.

In some embodiments the double stranded molecule disclosed herein includes a combination of the following modifications
the antisense strand includes a DNA, TNA, a 2′5′ nucleotide or a mirror nucleotide in at least one of positions 5, 6, 7, 8, or 9 from the 5′ terminus;
the sense strand includes at least one of a TNA, a 2′5′ nucleotide and a pseudoUridine in positions 9 or 10 from the 5′ terminus; and
one or more pyrimidine ribonucleotides are 2′ modified in the sense strand, the antisense strand or both the sense strand and the antisense strand.

In some embodiments the double stranded molecule disclosed herein includes a combination of the following modifications
the antisense strand includes a DNA, 2′5′ nucleotide or a mirror nucleotide in at least one of positions 5, 6, 7, 8, or 9 from the 5′ terminus;
the sense strand includes 4, 5, or 6 consecutive 2′5′ nucleotides at the 3′ penultimate or 3′ terminal positions; and
one or more pyrimidine ribonucleotides are 2′ sugar modified in the sense strand, the antisense strand or both the sense strand and the antisense strand.

In some embodiments of Structure (A1) and/or (A2) (N)y includes at least one unconventional moiety selected from a mirror nucleotide, a 2′5′ ribonucleotide and a TNA. In some embodiments the unconventional moiety is a mirror nucleotide. In various embodiments the mirror nucleotide is selected from an L-ribonucleotide (L-RNA) and an L-deoxyribonucleotide (L-DNA). In preferred embodiments the mirror nucleotide is L-DNA. In certain embodiments the sense strand comprises an unconventional moiety in position 9 or 10 (from the 5′ terminus). In preferred embodiments the sense strand includes an unconventional moiety in position 9 (from the 5′ terminus). In some embodiments the sense strand is 19 nucleotides in length and comprises 4, 5, or 6 consecutive unconventional moieties in positions 15 (from the 5′ terminus). In some embodiments the sense strand includes 4 consecutive 2′5′ ribonucleotides in positions 15, 16, 17, and 18. In some embodiments the sense strand includes 5 consecutive 2′5′ ribonucleotides in positions 15, 16, 17, 18 and 19. In various embodiments the sense strand further comprises Z′. In some embodiments Z′ includes a C3OH moiety or a C3Pi moiety.

In some embodiments of Structure (A1) and/or (A2) (N)y comprises at least one unconventional moiety selected from a mirror nucleotide and a nucleotide joined to an adjacent nucleotide by a 2′-5′ internucleotide phosphate bond. In some embodiments the unconventional moiety is a mirror nucleotide. In various embodiments the mirror nucleotide is selected from an L-ribonucleotide (L-RNA) and an L-deoxyribonucleotide (L-DNA). In preferred embodiments the mirror nucleotide is L-DNA.

In some embodiments of Structure A1 (N′)y comprises at least one L-DNA moiety. In some embodiments x=y=19 and (N′)y consists of unmodified ribonucleotides at positions 1-17 and 19 and one L-DNA at the 3′ penultimate position (position 18). In other embodiments x=y=19 and (N′)y consists of unmodified ribonucleotides at position 1-16 and 19 and two consecutive L-DNA nucleotides at the 3′ penultimate position (positions 17 and 18). In various embodiments the unconventional moiety is a nucleotide joined to an adjacent nucleotide by a 2′-5′ internucleotide phosphate linkage. According to various embodiments (N′)y comprises 2, 3, 4, 5, or 6 consecutive ribonucleotides at the 3′ terminus linked by 2′-5′ internucleotide linkages. In one embodiment, four consecutive ribonucleotides at the 3′ terminus of (N′)y are joined by three 2′-5′ phosphodiester bonds. In one embodiment, five consecutive ribonucleotides at the 3′ terminus of (N′)y are joined by four 2′-5′ phosphodiester bonds. In some embodiments, wherein one or more of the 2′-5′ ribonucleotides form a 2′-5′ phosphodiester bonds the nucleotide further comprises a 3′-O-methyl (3′OMe) sugar modification. In some embodiments the 3′ terminal nucleotide of (N′)y comprises a 3′OMe sugar modification. In certain embodiments x=y=19 and (N′)y comprises two or more consecutive nucleotides at positions 15, 16, 17, 18 and 19 which are joined to an adjacent nucleotide by a 2′-5′ internucleotide bond. In various embodiments, the nucleotide forming the 2′-5′ internucleotide bond comprises a ribonucleotide. In preferred embodiments the 2′-5′ internucleotide bond is a phosphosdiester internucleotide bond. In various embodiments the nucleotide forming the 2′-5′ internucleotide bond comprises a 3′ deoxyribose nucleotide or a 3′ methoxy nucleotide. In various embodiments, the ribonucleotide forming the 2′-5′ internucleotide bond comprises a 3′ deoxyribose ribonucleotide or a 3′ methoxy ribonucleotide. In some embodiments x=y=19 and (N′)y comprises nucleotides joined to the adjacent nucleotide by a 2′-5′ internucleotide bond between positions 15-16, 16-17 and 17-18 or between positions 16-17, 17-18 and 18-19. In some embodiments x=y=19 and (N′)y comprises nucleotides joined to the adjacent nucleotide by a 2′-5′ internucleotide bond between positions 16-17 and 17-18 or between positions 17-18 and 18-19 or between positions 15-16 and 17-18. In various embodiments, the nucleotides forming the 2′-5′ internucleotide bond comprise ribonucleotides. In various embodiments, the nucleotides forming the 2′-5′ internucleotide bond are ribonucleotides. In other embodiments the pyrimidine ribonucleotides (rU, rC) in (N′)y are substituted with a ribonucleotide joined to the adjacent ribonucleotide by a 2′-5′ internucleotide bond.

In some embodiments of Structure (A2), (N)y comprises at least one L-DNA moiety. In some embodiments x=y=18 and N²—(N′)y, consists of unmodified ribonucleotides at positions 1-17 and 19 and one L-DNA at the 3′ penultimate position (position 18). In other embodiments x=y=18 and N²—(N′)y consists of unmodified ribonucleotides at position 1-16 and 19 and two consecutive L-DNA at the 3′ penultimate position (positions 17 and 18). In various embodiments the unconventional moiety is a nucleotide joined to an adjacent nucleotide by a 2′-5′ internucleotide phosphate linkage. According to various embodiments N²—(N′)y comprises 2, 3, 4, 5, or 6 consecutive ribonucleotides at the 3′ terminus linked by 2′-5′ internucleotide linkages. In one embodiment, four consecutive ribonucleotides at the 3′ terminus of N²—(N′)y are joined by three 2′-5′ phosphodiester bonds, wherein one or more of the 2′-5′ ribonucleotides which form the 2′-5′ phosphodiester bonds further comprises a 3′-O-methyl (3′OMe) sugar modification. In some embodiments the 3′ terminal ribonucleotide of N²—(N′)y comprises a 2′OMe sugar modification. In certain embodiments x=y=18 and N²—(N′)y comprises two or more consecutive nucleotides at positions 15, 16, 17, 18 and 19 joined to an adjacent nucleotide by a 2′-5′ internucleotide bond. In various embodiments the nucleotide forming the 2′-5′ internucleotide bond comprises a 3′ deoxyribose nucleotide or a 3′ methoxy nucleotide. In various embodiments, the ribonucleotide forming the 2′-5′ internucleotide bond comprises a 3′ deoxyribose ribonucleotide or a 3′ methoxy ribonucleotide. In some embodiments x=y=18 and N²—(N′)y comprises nucleotides joined to the adjacent nucleotide by a 2′-5′ internucleotide bond between positions 16-17 and 17-18 or between positions 17-18 and 18-19 or between positions 15-16 and 17-18. In various embodiments, the nucleotides forming the 2′-5′ internucleotide bond comprise ribonucleotides. In various embodiments, the nucleotides forming the 2′-5′ internucleotide bond are ribonucleotides. In other embodiments a pyrimidine ribonucleotide (rU, rC) in (N′)y comprises a ribonucleotide joined to the adjacent ribonucleotide by a 2′-5′ internucleotide bond.

In further embodiments of Structures (A1) and/or (A2) (N′)y comprises 1-8 modified ribonucleotides wherein the modified ribonucleotide is a deoxyribose (DNA) nucleotide. In certain embodiments (N′)y comprises 1, 2, 3, 4, 5, 6, 7, or up to 8 DNA moieties.

In a presently preferred embodiment the inhibitor provided herein is a synthetic, chemically modified double stranded RNA (dsRNA) compound that down-regulates HES1 expression and includes an oligonucleotide pair selected from Table I. In the presently preferred embodiments the inhibitor provided herein is a synthetic, chemically modified double stranded RNA (dsRNA) compound that down-regulates HES5 expression and includes an oligonucleotide pair selected from Table II. In the presently preferred embodiments the inhibitor provided herein is a synthetic, chemically modified double stranded RNA (dsRNA) compound that down-regulates HEY1 expression and includes an oligonucleotide pair selected from Table III. In the presently preferred embodiments the inhibitor provided herein is a synthetic, chemically modified double stranded RNA (dsRNA) compound that down-regulates HEY2 expression and includes an oligonucleotide pair selected from Table IV. In the presently preferred embodiments the inhibitor provided herein is a synthetic, chemically modified double stranded RNA (dsRNA) compound that down-regulates ID1 expression and includes an oligonucleotide pair selected from Table V. In the presently preferred embodiments the inhibitor provided herein is a synthetic, chemically modified double stranded RNA (dsRNA) compound that down-regulates ID2 expression and includes an oligonucleotide pair selected from Table VI. In the presently preferred embodiments the inhibitor provided herein is a synthetic, chemically modified double stranded RNA (dsRNA) compound that down-regulates ID3 expression and includes an oligonucleotide pair selected from Table VII. In the presently preferred embodiments the inhibitor provided herein is a synthetic, chemically modified double stranded RNA (dsRNA) compound that down-regulates CDKN1B expression and includes an oligonucleotide pair selected from Table VIII. In the presently preferred embodiments the inhibitor provided herein is a synthetic, chemically modified double stranded RNA (dsRNA) compound that down-regulates NOTCH1 expression and includes an oligonucleotide pair selected from Table IX. Tables I-IX are provided herein below.

TABLE I
Selected HES1 dsRNA

| | SEQ | | SEQ | | |
|---|---|---|---|---|---|
| dsRNA | ID | | ID | | |
| Name | NO: | Sense strand(5′ > 3′) | NO: | Antisense strand (5′ > 3′) | Type |
| HES1_12 | 26667 | GCCAGCUGAUAUAAUGGAA | 26679 | UUCCAUUAUAUCAGCUGGC | 18 + 1 |
| HES1_13 | 26668 | GCCAGUGUCAACACGACAA | 26680 | UUGUCGUGUUGACACUGGC | 18 + 1 |
| HES1_14 | 26669 | CAGCGAGUGCAUGAACGAA | 26681 | UUCGUUCAUGCACUCGCUG | 18 + 1 |
| HES1_16 | 26670 | GAACGAGGUGACCCGCUUA | 26682 | UAAGCGGGUCACCUCGUUC | 18 + 1 |
| HES1_19 | 26671 | CCAGUGUCAACACGACACA | 26683 | UGUGUCGUGUUGACACUGG | 18 + 1 |
| HES1_20 | 26672 | CGAGUGCAUGAACGAGGUA | 26684 | UACCUCGUUCAUGCACUCG | 18 + 1 |
| HES1_21 | 26673 | UGUCAACACGACACCGGAA | 26685 | UUCCGGUGUCGUGUUGACA | 18 + 1 |
| HES1_22 | 26674 | CAGUGUCAACACGACACCA | 26686 | UGGUGUCGUGUUGACACUG | 18 + 1 |
| HES1_24 | 26675 | GGCGGACUCCAUGUGGAGA | 26687 | UCUCCACAUGGAGUCCGCC | 18 + 1 |
| HES1_28 | 26676 | CGGAUAAACCAAAGACAGA | 26688 | UCUGUCUUUGGUUUAUCCG | 18 + 1 |
| HES1_33 | 26677 | AGUGCAUGAACGAGGUGAA | 26689 | UUCACCUCGUUCAUGCACU | 18 + 1 |
| HES1_36 | 26678 | CAGCGAGUGCAUGAACGAU | 26690 | AUCGUUCAUGCACUCGCUG | 18 + 1 |
| HES1_10 | 26691 | GUAUUAAGUGACUGACCAU | 26699 | AUGGUCAGUCACUUAAUAC | 19 |
| HES1_11 | 26692 | GAAAACACUGAUUUUGGAU | 26700 | AUCCAAAAUCAGUGUUUUC | 19 |
| HES1_15 | 26693 | ACUGCAUGACCCAGAUCAA | 26701 | UUGAUCUGGGUCAUGCAGU | 19 |
| HES1_17 | 26694 | AGCCAGUGUCAACACGACA | 26702 | UGUCGUGUUGACACUGGCU | 19 |
| HES1_18 | 26695 | GUGUCAACACGACACCGGA | 26703 | UCCGGUGUCGUGUUGACAC | 19 |
| HES1_26 | 26696 | CAGUGAAGCACCUCCGGAA | 26704 | UUCCGGAGGUGCUUCACUG | 19 |
| HES1_27 | 26697 | CAUGGAGAAAAGACGAAGA | 26705 | UCUUCGUCUUUUCUCCAUG | 19 |
| HES1_35 | 26698 | CAGCUGAUAUAAUGGAGAA | 26706 | UUCUCCAUUAUAUCAGCUG | 19 |

In some embodiments the dsRNA includes an antisense strand and a sense strand having the HES1_{—}10 antisense and sense sequences set forth in SEQ ID NOS:26699 and 266901. In some embodiments the dsRNA includes an antisense strand and a sense strand having the HES1_{—}11 antisense and sense sequences set forth in SEQ ID NOS:26700 and 26692. In some embodiments the dsRNA includes an antisense strand and a sense strand having the HES1_{—}12 antisense and sense sequences set forth in SEQ ID NOS:26679 and 26667. In some embodiments the dsRNA includes an antisense strand and a sense strand having the HES1_{—}13 antisense and sense sequences set forth in SEQ ID NOS:26680 and 26668. In some embodiments the dsRNA includes an antisense strand and a sense strand having the HES1_{—}14 antisense and sense sequences set forth in SEQ ID NOS:26681 and 26669. In some embodiments the dsRNA includes an antisense strand and a sense strand having the HES1_{—}15 antisense and sense sequences set forth in SEQ ID NOS:26701 and 26693. In some embodiments the dsRNA includes an antisense strand and a sense strand having the HES1_{—}16 antisense and sense sequences set forth in SEQ ID NOS:26682 and 26670. In some embodiments the dsRNA includes an antisense strand and a sense strand having the HES1_{—}17 antisense and sense sequences set forth in SEQ ID NOS:26702 and 26694. In some embodiments the dsRNA includes an antisense strand and a sense strand having the HES1_{—}18 antisense and sense sequences set forth in SEQ ID NOS:26703 and 26695. In some embodiments the dsRNA includes an antisense strand and a sense strand having the HES1_{—}19 antisense and sense sequences set forth in SEQ ID NOS:26683 and 26671. In some embodiments the dsRNA includes an antisense strand and a sense strand having the HES1_{—}20 antisense and sense sequences set forth in SEQ ID NOS:26684 and 26672. In some embodiments the dsRNA includes an antisense strand and a sense strand having the HES1_{—}21 antisense and sense sequences set forth in SEQ ID NOS:26685 and 26673. In some embodiments the dsRNA includes an antisense strand and a sense strand having the HES1_{—}22 antisense and sense sequences set forth in SEQ ID NOS:26686 and 26674. In some embodiments the dsRNA includes an antisense strand and a sense strand having the HES1_{—}24 antisense and sense sequences set forth in SEQ ID NOS:26687 and 26675. In some embodiments the dsRNA includes an antisense strand and a sense strand having the HES1_{—}26 antisense and sense sequences set forth in SEQ ID NOS:26704 and 26696. In some embodiments the dsRNA includes an antisense strand and a sense strand having the HES1_{—}27 antisense and sense sequences set forth in SEQ ID NOS:26705 and 26697. In some embodiments the dsRNA includes an antisense strand and a sense strand having the HES1_{—}28 antisense and sense sequences set forth in SEQ ID NOS:26688 and 26676. In some embodiments the dsRNA includes an antisense strand and a sense strand having the HES1_{—}33 antisense and sense sequences set forth in SEQ ID NOS:26689 and 26677. In some embodiments the dsRNA includes an antisense strand and a sense strand having the HES1_{—}36 antisense and sense sequences set forth in SEQ ID NOS:26690 and 26678.

In some preferred embodiments the dsRNA includes an antisense strand and a sense strand having the HES1_{—}14 antisense and sense sequences set forth in SEQ ID NOS:26681 and 26669. In some preferred embodiments the dsRNA includes an antisense strand and a sense strand having the HES1_{—}36 antisense and sense sequences set forth in SEQ ID NOS:26690 and 26678.

All positions given are 5′>3′ on the sense strand and on the antisense strand.

In some embodiments the dsRNA includes an antisense strand and a sense strand having the HES1_{—}14 antisense and sense sequences set forth in SEQ ID NOS:26,681 and 26,669 and includes the following modifications:

The sense strand includes 5 consecutive 2′5′ ribonucleotides in positions 15, 16, 17, 18, and 19, a capping moiety covalently attached at the 5 terminus, and a C3Pi moiety covalently attached at the 3′ terminus; and the antisense strand includes 2′OMe sugar modified ribonucleotides in positions 1, 3, 9, 11, 14, 15, and 18, optionally a 2′5′ ribonucleotide in position 7 and a C3Pi-C3OH moiety covalently attached to the 3′ terminus; or

The sense strand includes 2′OMe sugar modified ribonucleotides in positions 1, 4, 8, 10, 12 and 16, a capping moiety covalently attached at the 5 terminus, and a C3Pi moiety covalently attached at the 3′ terminus; and the antisense strand includes 2′OMe sugar modified ribonucleotides in positions 1, 3, 9, 11, 14, 15, and 18, optionally a 2′5′ ribonucleotide in position 7 and a C3Pi-C3OH moiety covalently attached to the 3′ terminus.

In preferred embodiments of dsRNA having the HES1_{—}14 sequence (antisense and sense sequences set forth in SEQ ID NOS:26681 and 26669) and modifications as provided above the capping moiety is an inverted abasic deoxyribonucleotide moiety.

In some embodiments, the dsRNA includes an antisense strand and a sense strand having the HES1_{—}36 antisense and sense sequences set forth in SEQ ID NOS:26690 and 26678 and includes the following modifications:

The sense strand includes 5 consecutive 2′5′ ribonucleotides in positions 15, 16, 17, 18, and 19, a capping moiety covalently attached at the 5 terminus, and a non-nucleotide moiety covalently attached at the 3′ terminus; and the antisense strand includes 2′OMe sugar modified ribonucleotides in positions 3, 9, 11 and 15, optionally a 2′5′ ribonucleotide in position 7 and a non-nucleotide moiety covalently attached to the 3′ terminus.

In preferred embodiments the sense strand includes 5 consecutive 2′5′ ribonucleotides in positions 15, 16, 17, 18, and 19, a capping moiety covalently attached at the 5 terminus, and a C3Pi moiety covalently attached at the 3′ terminus; and the antisense strand includes 2′OMe sugar modified ribonucleotides in positions 3, 9, 11 and 15, and a C3Pi-C3OH moiety covalently attached to the 3′ terminus. In preferred embodiments the capping moiety is an inverted abasic deoxyribonucleotide moiety.

TABLE II
Selected HES5 dsRNA

| | SEQ | | | | |
|---|---|---|---|---|---|
| dsRNA | ID | | SEQ ID | Antisense strand | |
| Name | NO: | Sense strand (5′ > 3′) | NO: | (5′ > 3′) | Type |
| HES5_19 | 26707 | GGAGUUCGCGCGGCACCAA | 26716 | UUGGUGCCGCGCGAACUCC | 18 + 1 |
| HES5_20 | 26708 | GCGACACGCAGAUGAAGCA | 26717 | UGCUUCAUCUGCGUGUCGC | 18 + 1 |
| HES5_22 | 26709 | CGGGCACAUUUGCCUUUUA | 26718 | UAAAAGGCAAAUGUGCCCG | 18 + 1 |
| HES5_23 | 26710 | CGCCAGCGACACGCAGAUA | 26719 | UAUCUGCGUGUCGCUGGCG | 18 + 1 |
| HES5_24 | 26711 | CCGACUGCGGAAGCCGGUA | 26720 | UACCGGCUUCCGCAGUCGG | 18 + 1 |
| HES5_26 | 26712 | GCGCGGCACCAGCCCAACA | 26721 | UGUUGGGCUGGUGCCGCGC | 18 + 1 |
| HES5_27 | 26713 | AACCGACUGCGGAAGCCGA | 26722 | UCGGCUUCCGCAGUCGGUU | 18 + 1 |
| HES5_28 | 26714 | CGACUGCGGAAGCCGGUGA | 26723 | UCACCGGCUUCCGCAGUCG | 18 + 1 |
| HES5_29 | 26715 | CGACACGCAGAUGAAGCUA | 26724 | UAGCUUCAUCUGCGUGUCG | 18 + 1 |
| HES5_10 | 26725 | CUGUAGAGGACUUUCUUCA | 26729 | UGAAGAAAGUCCUCUACAG | 19 |
| HES5_21 | 26726 | GCCAGCGACACGCAGAUGA | 26730 | UCAUCUGCGUGUCGCUGGC | 19 |
| HES5_25 | 26727 | GCGACACGCAGAUGAAGCU | 26731 | AGCUUCAUCUGCGUGUCGC | 19 |
| HES5_8 | 26728 | GGGUUCUAUGAUAUUUGUA | 26732 | UACAAAUAUCAUAGAACCC | 19 |

In some embodiments the dsRNA includes an antisense strand and a sense strand having the HES5_{—}8 antisense and sense sequences set forth in SEQ ID NOS:26732 and 26728. In some embodiments the dsRNA includes an antisense strand and a sense strand having the HES5_{—}10 antisense and sense sequences set forth in SEQ ID NOS:26729 and 26725. In some embodiments the dsRNA includes an antisense strand and a sense strand having the HES5_{—}19 antisense and sense sequences set forth in SEQ ID NOS:26716 and 26707. In some embodiments the dsRNA includes an antisense strand and a sense strand having the HES5_{—}20 antisense and sense sequences set forth in SEQ ID NOS:26717 and 26708. In some embodiments the dsRNA includes an antisense strand and a sense strand having the HES5_{—}21 antisense and sense sequences se forth in SEQ ID NOS:26730 and 26726. In some embodiments the dsRNA includes an antisense strand and a sense strand having the HES5_{—}22 antisense and sense sequences set forth in SEQ ID NOS:26718 and 26709. In some embodiments the dsRNA includes an antisense strand and a sense strand having the HES5_{—}23 antisense and sense sequences set forth in SEQ ID NOS:26719 and 26710. In some embodiments the dsRNA includes an antisense strand and a sense strand having the HES5_{—}24 antisense and sense sequences set forth in SEQ ID NOS:26720 and 26711. In some embodiments the dsRNA includes an antisense strand and a sense strand having the HES5_{—}25 antisense and sense sequences set forth in SEQ ID NOS:26731 and 26727. In some embodiments the dsRNA includes an antisense strand and a sense strand having the HES5_{—}26 antisense and sense sequences set forth in SEQ ID NOS:26721 and 26712. In some embodiments the dsRNA includes an antisense strand and a sense strand having the HES5_{—}27 antisense and sense sequences set forth in SEQ ID NOS:26722 and 26713. In some embodiments the dsRNA includes an antisense strand and a sense strand having the HES5_{—}28 antisense and sense sequences set forth in SEQ ID NOS:26723 and 26714. In some embodiments the dsRNA includes an antisense strand and a sense strand having the HES5_{—}29 antisense and sense sequences set forth in SEQ ID NOS:26724 and 26715. In some preferred embodiments the dsRNA includes an antisense strand and a sense strand having the HES5_{—}8 antisense and sense sequences set forth in SEQ ID NOS:26732 and 26728.

TABLE III
Selected HEY1 dsRNA

| | SEQ | | | | |
|---|---|---|---|---|---|
| dsRNA | ID | Sense strand | SEQ ID | Antisense strand | |
| Name | NO: | (5′ > 3′) | NO: | (5′ > 3′) | Type |
| HEY1_1 | 26733 | GUUUGUCUGAGCUGAGAAA | 26747 | UUUCUCAGCUCAGACAAAC | 18 + 1 |
| HEY1_2 | 26734 | GAGCUGAGAAGGCUGGUAA | 26748 | UUACCAGCCUUCUCAGCUC | 18 + 1 |
| HEY1_3 | 26735 | UGAGCAUCAUUGAAGGACA | 26749 | UGUCCUUCAAUGAUGCUCA | 18 + 1 |
| HEY1_5 | 26736 | UGAGAAGCGCCGACGAGAA | 26750 | UUCUCGUCGGCGCUUCUCA | 18 + 1 |
| HEY1_6 | 26737 | ACAGUUUGUCUGAGCUGAA | 26751 | UUCAGCUCAGACAAACUGU | 18 + 1 |
| HEY1_9 | 26738 | UGUCUGAGCUGAGAAGGCA | 26752 | UGCCUUCUCAGCUCAGACA | 18 + 1 |
| HEY1_10 | 26739 | GAGAAGCGCCGACGAGACA | 26753 | UGUCUCGUCGGCGCUUCUC | 18 + 1 |
| HEY1_12 | 26740 | GAAGCGCCGACGAGACCGA | 26754 | UCGGUCUCGUCGGCGCUUC | 18 + 1 |
| HEY1_14 | 26741 | GCAUCUCAACAACUACGCA | 26755 | UGCGUAGUUGUUGAGAUGC | 18 + 1 |
| HEY1_16 | 26742 | AGCCCUAUAGACCUUGGGA | 26756 | UCCCAAGGUCUAUAGGGCU | 18 + 1 |
| HEY1_18 | 26743 | UGCAAACCUUGGCAAGCCA | 26757 | UGGCUUGCCAAGGUUUGCA | 18 + 1 |
| HEY1_19 | 26744 | GGCCUCGGACACAUUCCCA | 26758 | UGGGAAUGUGUCCGAGGCC | 18 + 1 |
| HEY1_21 | 26745 | CCAGCGGGAAGCCGCGAGA | 26759 | UCUCGCGGCUUCCCGCUGG | 18 + 1 |
| HEY1_22 | 26746 | UGGACUAUCGGAGUUUGGA | 26760 | UCCAAACUCCGAUAGUCCA | 18 + 1 |
| HEY1_4 | 26761 | AGUUUGUCUGAGCUGAGAA | 26770 | UUCUCAGCUCAGACAAACU | 19 |
| HEY1_7 | 26762 | UGAGCAUCAUUGAAGGACU | 26771 | AGUCCUUCAAUGAUGCUCA | 19 |
| HEY1_8 | 26763 | CUGAGAAGGCUGGUACCCA | 26772 | UGGGUACCAGCCUUCUCAG | 19 |
| HEY1_11 | 26764 | GUUUGUCUGAGCUGAGAAG | 26773 | CUUCUCAGCUCAGACAAAC | 19 |
| HEY1_13 | 26765 | AACAGUUUGUCUGAGCUGA | 26774 | UCAGCUCAGACAAACUGUU | 19 |
| HEY1_15 | 26766 | UGUCUGAGCUGAGAAGGCU | 26775 | AGCCUUCUCAGCUCAGACA | 19 |
| HEY1_17 | 26767 | UGCUAUGGACUAUCGGAGU | 26776 | ACUCCGAUAGUCCAUAGCA | 19 |
| HEY1_20 | 26768 | GAGCUGAGAAGGCUGGUAC | 26777 | GUACCAGCCUUCUCAGCUC | 19 |
| HEY1_23 | 26769 | UGCGGACGAGAAUGGAAAC | 26778 | GUUUCCAUUCUCGUCCGCA | 19 |

In some embodiments the dsRNA includes an antisense strand and a sense strand having the HEY1_{—}1 antisense and sense sequences set forth in SEQ ID NOS:26747 and 26733. In some embodiments the dsRNA includes an antisense strand and a sense strand having the HEY1_{—}2 antisense and sense sequences set forth in SEQ ID NOS:26748 and 26734. In some embodiments the dsRNA includes an antisense strand and a sense strand having the HEY1_{—}3 antisense and sense sequences set forth in SEQ ID NOS:26749 and 26735. In some embodiments the dsRNA includes an antisense strand and a sense strand having the HEY1_{—}4 antisense and sense sequences set forth in SEQ ID NOS:26770 and 26761. In some embodiments the dsRNA includes an antisense strand and a sense strand having the HEY1_{—}5 antisense and sense sequences set forth in SEQ ID NOS:26750 and 26736. In some embodiments the dsRNA includes an antisense strand and a sense strand having the HEY1_{—}6 antisense and sense sequences set forth in SEQ ID NOS:26751 and 26737. In some embodiments the dsRNA includes an antisense strand and a sense strand having the HEY1_{—}7 antisense and sense sequences set forth in SEQ ID NOS:26771 and 26762. In some embodiments the dsRNA includes an antisense strand and a sense strand having the HEY1_{—}8 antisense and sense sequences set forth in SEQ ID NOS:26772 and 26763. In some embodiments the dsRNA includes an antisense strand and a sense strand having the HEY1_{—}9 antisense and sense sequences set forth in SEQ ID NOS:26752 and 26738. In some embodiments the dsRNA includes an antisense strand and a sense strand having the HEY1_{—}10 antisense and sense sequences set forth in SEQ ID NOS:26753 and 26739. In some embodiments the dsRNA includes an antisense strand and a sense strand having the HEY1_{—}11 antisense and sense sequences set forth in SEQ ID NOS:26773 and 26764. In some embodiments the dsRNA includes an antisense strand and a sense strand having the HEY1_{—}12 antisense and sense sequences set forth in SEQ ID NOS:26754 and 26740. In some embodiments the dsRNA includes an antisense strand and a sense strand having the HEY1_{—}13 antisense and sense sequences set forth in SEQ ID NOS:26774 and 267658. In some embodiments the dsRNA includes an antisense strand and a sense strand having the HEY1_{—}14 antisense and sense sequences set forth in SEQ ID NOS:26755 and 26741. In some embodiments the dsRNA includes an antisense strand and a sense strand having the HEY1_{—}15 antisense and sense sequences set forth in SEQ ID NOS:26775 and 26766. In some embodiments the dsRNA includes an antisense strand and a sense strand having the HEY1_{—}16 antisense and sense sequences set forth in SEQ ID NOS:26756 and 26742. In some embodiments the dsRNA includes an antisense strand and a sense strand having the HEY1_{—}17 antisense and sense sequences set forth in SEQ ID NOS:26776 and 26767. In some embodiments the dsRNA includes an antisense strand and a sense strand having the HEY1_{—}18 antisense and sense sequences set forth in SEQ ID NOS:26757 and 26743. In some embodiments the dsRNA includes an antisense strand and a sense strand having the HEY1_{—}19 antisense and sense sequences set forth in SEQ ID NOS:267582 and 26744. In some embodiments the dsRNA includes an antisense strand and a sense strand having the HEY1_{—}20 antisense and sense sequences set forth in SEQ ID NOS:26777 and 26768. In some embodiments the dsRNA includes an antisense strand and a sense strand having the HEY1_{—}21 antisense and sense sequences set forth in SEQ ID NOS:26759 and 26745. In some embodiments the dsRNA includes an antisense strand and a sense strand having the HEY1_{—}22 antisense and sense sequences set forth in SEQ ID NOS:26760 and 26746. In some embodiments the dsRNA includes an antisense strand and a sense strand having the HEY1_{—}23 antisense and sense sequences set forth in SEQ ID NOS:26778 and 26769.

TABLE IV
Selected HEY2 dsRNA

| | SEQ | | | | |
|---|---|---|---|---|---|
| dsRNA | ID | Sense strand | SEQ ID | AntiSense strand | |
| Name | NO: | (5′ > 3′) | NO: | (5′ > 3′) | Type |
| HEY2_1 | 26779 | GGGAGCGAGAACAAUUACA | 26782 | UGUAAUUGUUCUCGCUCCC | 18 + 1 |
| HEY2_2 | 26780 | GGGUAAAGGCUACUUUGAA | 26783 | UUCAAAGUAGCCUUUACCC | 18 + 1 |
| HEY2_5 | 26781 | GAAAAGGCGUCGGGAUCGA | 26784 | UCGAUCCCGACGCCUUUUC | 18 + 1 |
| HEY2_3 | 26785 | GGGUAAAGGCUACUUUGAC | 26787 | GUCAAAGUAGCCUUUACCC | 19 |
| HEY2_4 | 26786 | CCAUGGCCCACCACCAUCA | 26788 | UGAUGGUGGUGGGCCAUGG | 19 |

In some embodiments the dsRNA includes an antisense strand and a sense strand having the HEY2_{—}1 antisense and sense sequences set forth in SEQ ID NOS:26782 and 26779. In some embodiments the dsRNA includes an antisense strand and a sense strand having the HEY2_{—}2 antisense and sense sequences set forth in SEQ ID NOS:26783 and 26780. In some embodiments the dsRNA includes an antisense strand and a sense strand having the HEY2_{—}3 antisense and sense sequences set forth in SEQ ID NOS:26785 and 26787. In some embodiments the dsRNA includes an antisense strand and a sense strand having the HEY2_{—}4 antisense and sense sequences set forth in SEQ ID NOS:26786 and 26788. In some embodiments the dsRNA includes an antisense strand and a sense strand having the HEY2_{—}5 antisense and sense sequences set forth in SEQ ID NOS:26784 and 26781.

In some preferred embodiments the dsRNA includes an antisense strand and a sense strand having the HEY2_{—}1 antisense and sense sequences set forth in SEQ ID NOS:26782 and 26779. In some preferred embodiments the dsRNA includes an antisense strand and a sense strand having the HEY2_{—}2 antisense and sense sequences set forth in SEQ ID NOS:26783 and 26780.

In some embodiments the dsRNA includes an antisense strand and a sense strand having the HEY2_{—}1 antisense and sense sequences set forth in SEQ ID NOS:26782 and 26779 and includes the following modifications:

The sense strand includes 2′OMe sugar modified ribonucleotides in positions 6 and 12, a capping moiety covalently attached at the 5 terminus, a non-nucleotide moiety covalently attached at the 3′ terminus and optionally 5 consecutive 2′5′ ribonucleotides in positions 15, 16, 17, 18, and 19 or 2′OMe sugar modified ribonucleotides in positions 16 and 18; and the antisense strand includes 2′OMe sugar modified ribonucleotides in positions 1, 3, 9, 11, 13, 15, 17 and 19, optionally a 2′5′ ribonucleotide in position 7 and a non-nucleotide moiety covalently attached to the 3′ terminus. In preferred embodiments the capping moiety is an inverted abasic deoxyribonucleotide moiety. In preferred embodiments the non-nucleotide moiety covalently attached at the 3′ end of the sense strand is a C3Pi moiety. In preferred embodiments the non-nucleotide moiety covalently attached at the 3′ end of the antisense strand is a C3PiC3OH or C3PiC3Pi moiety. In some embodiments the dsRNA includes an antisense strand and a sense strand having the HEY2_{—}2 antisense and sense sequences set forth in SEQ ID NOS:26783 and 26780 and includes the following modifications:

The sense strand includes 2′OMe sugar modified ribonucleotides in positions 4, 11 and 13, an inverted abasic deoxyribonucleotide moiety covalently attached at the 5 terminus, a non-nucleotide moiety covalently attached at the 3′ terminus and optionally 5 consecutive 2′5′ ribonucleotides in positions 15, 16, 17, 18, and 19 or a 2′OMe sugar modified ribonucleotide in position 16; and the antisense strand includes 2′OMe sugar modified ribonucleotides in positions 1, 3, 8, 11, 13, 15, 17 and 19, optionally a 2′5′ ribonucleotide in position 7 and a non-nucleotide moiety covalently attached to the 3′ terminus. In preferred embodiments the capping moiety is an inverted abasic deoxyribonucleotide moiety. In preferred embodiments the non-nucleotide moiety covalently attached at the 3′end of the sense strand is a C3Pi moiety. In preferred embodiments the non-nucleotide moiety covalently attached at the 3′ end of the antisense strand is a C3PiC3OH or C3PiC3Pi moiety.

TABLE V
Selected ID1 dsRNA

| | | | SEQ | | |
|---|---|---|---|---|---|
| dsRNA | SEQ ID | | ID | AntiSense strand | |
| Name | NO: | Sense strand (5′ > 3′) | NO: | (5′ > 3′) | Type |
| ID1_10 | 26789 | AGCACGUCAUCGACUACAA | 26799 | UUGUAGUCGAUGACGUGCU | 18 + 1 |
| ID1_11 | 26790 | GCACGUCAUCGACUACAUA | 26800 | UAUGUAGUCGAUGACGUGC | 18 + 1 |
| ID1_13 | 26791 | CCAGCACGUCAUCGACUAA | 26801 | UUAGUCGAUGACGUGCUGG | 18 + 1 |
| ID1_14 | 26792 | UGCUCUACGACAUGAACGA | 26802 | UCGUUCAUGUCGUAGAGCA | 18 + 1 |
| ID1_15 | 26793 | GACGAUCGCAUCUUGUGUA | 26803 | UACACAAGAUGCGAUCGUC | 18 + 1 |
| ID1_18 | 26794 | GCUCUACGACAUGAACGGA | 26804 | UCCGUUCAUGUCGUAGAGC | 18 + 1 |
| ID1_19 | 26795 | GAUCGCAUCUUGUGUCGCA | 26805 | UGCGACACAAGAUGCGAUC | 18 + 1 |
| ID1_20 | 26796 | UCUACGACAUGAACGGCUA | 26806 | UAGCCGUUCAUGUCGUAGA | 18 + 1 |
| ID1_21 | 26797 | UCAAGGAGCUGGUGCCCAA | 26807 | UUGGGCACCAGCUCCUUGA | 18 + 1 |
| ID1_22 | 26798 | ACGAUCGCAUCUUGUGUCA | 26808 | UGACACAAGAUGCGAUCGU | 18 + 1 |
| ID1_12 | 26809 | AGCACGUCAUCGACUACAU | 26813 | UUGUAGUCGAUGACGUGCU | 19 |
| ID1_16 | 26810 | CGCAUCUUGUGUCGCUGAA | 26814 | UUCAGCGACACAAGAUGCG | 19 |
| ID1_17 | 26811 | CACGUCAUCGACUACAUCA | 26815 | UGAUGUAGUCGAUGACGUG | 19 |
| ID1_23 | 26812 | CAGCACGUCAUCGACUACA | 26816 | UGUAGUCGAUGACGUGCUG | 19 |

In some embodiments the dsRNA includes an antisense strand and a sense strand having the ID1_{—}10 antisense and sense sequences set forth in SEQ ID NOS:26799 and 26789. In some embodiments the dsRNA includes an antisense strand and a sense strand having the ID1_{—}11 antisense and sense sequences set forth in SEQ ID NOS:26800 and 26790. In some embodiments the dsRNA includes an antisense strand and a sense strand having the ID1_{—}12 antisense and sense sequences set forth in SEQ ID NOS:26813 and 26809. In some embodiments the dsRNA includes an antisense strand and a sense strand having the ID1_{—}13 antisense and sense sequences set forth in SEQ ID NOS:26801 and 26791. In some embodiments the dsRNA includes an antisense strand and a sense strand having the ID1_{—}14 antisense and sense sequences set forth in SEQ ID NOS:26802 and 26792. In some embodiments the dsRNA includes an antisense strand and a sense strand having the ID1_{—}15 antisense and sense sequences set forth in SEQ ID NOS:26803 and 26793. In some embodiments the dsRNA includes an antisense strand and a sense strand having the ID1_{—}16 antisense and sense sequences set forth in SEQ ID NOS:26814 and 26810. In some embodiments the dsRNA includes an antisense strand and a sense strand having the ID1_{—}17 antisense and sense sequences set forth in SEQ ID NOS:26815 and 26811. In some embodiments the dsRNA includes an antisense strand and a sense strand having the ID1_{—}18 antisense and sense sequences set forth in SEQ ID NOS:26804 and 26794. In some embodiments the dsRNA includes an antisense strand and a sense strand having the ID1_{—}19 antisense and sense sequences set forth in SEQ ID NOS:26805 and 26795. In some embodiments the dsRNA includes an antisense strand and a sense strand having the ID1_{—}20 antisense and sense sequences set forth in SEQ ID NOS:26806 and 26796. In some embodiments the dsRNA includes an antisense strand and a sense strand having the ID1_{—}21 antisense and sense sequences set forth in SEQ ID NOS:26807 and 26797. In some embodiments the dsRNA includes an antisense strand and a sense strand having the ID1_{—}22 antisense and sense sequences set forth in SEQ ID NOS:26808 and 26798. In some embodiments the dsRNA includes an antisense strand and a sense strand having the ID1_{—}23 antisense and sense sequences set forth in SEQ ID NOS:26816 and 26812.

TABLE VI
Selected ID2 duplexes

| | | | SEQ | | |
|---|---|---|---|---|---|
| dsRNA | SEQ ID | Sense strand | ID | | |
| Name | NO: | (5′ > 3′) | NO: | AntiSense strand (5′ > 3′) | Type |
| ID2_11 | 26817 | GGAGCGUGAAUACCAGAAA | 26821 | UUUCUGGUAUUCACGCUCC | 18 + 1 |
| ID2_13 | 26818 | AAAGGUGGAGCGUGAAUAA | 26822 | UUAUUCACGCUCCACCUUU | 18 + 1 |
| ID2_14 | 26819 | CAGCCUGUCGGACCACAGA | 26823 | UCUGUGGUCCGACAGGCUG | 18 + 1 |
| ID2_18 | 26820 | GAACCAGGCGUCCAGGACA | 26824 | UGUCCUGGACGCCUGGUUC | 18 + 1 |
| ID2_12 | 26825 | CAAAGGUGGAGCGUGAAUA | 26829 | UAUUCACGCUCCACCUUUG | 19 |
| ID2_15 | 26826 | UGGAGCGUGAAUACCAGAA | 26830 | UUCUGGUAUUCACGCUCCA | 19 |
| ID2_16 | 26827 | GGAGCGUGAAUACCAGAAG | 26831 | CUUCUGGUAUUCACGCUCC | 19 |
| ID2_17 | 26828 | AGAUCGCCCUGGACUCGCA | 26832 | UGCGAGUCCAGGGCGAUCU | 19 |

In some embodiments the dsRNA includes an antisense strand and a sense strand having the ID2_{—}11 antisense and sense sequences set forth in SEQ ID NOS:26821 and 26817. In some embodiments the dsRNA includes an antisense strand and a sense strand having the ID2_{—}12 antisense and sense sequences set forth in SEQ ID NOS:26829 and 26825. In some embodiments the dsRNA includes an antisense strand and a sense strand having the ID2_{—}13 antisense and sense sequences set forth in SEQ ID NOS:26822 and 26818. In some embodiments the dsRNA includes an antisense strand and a sense strand having the ID2_{—}14 antisense and sense sequences set forth in SEQ ID NOS:26823 and 268192. In some embodiments the dsRNA includes an antisense strand and a sense strand having the ID2_{—}15 antisense and sense sequences set forth in SEQ ID NOS:26830 and 26826. In some embodiments the dsRNA includes an antisense strand and a sense strand having the ID2_{—}16 antisense and sense sequences set forth in SEQ ID NOS:26831 and 26827. In some embodiments the dsRNA includes an antisense strand and a sense strand having the ID2_{—}17 antisense and sense sequences set forth in SEQ ID NOS:26832 and 26828. In some embodiments the dsRNA includes an antisense strand and a sense strand having the ID2_{—}18 antisense and sense sequences set forth in SEQ ID NOS:26824 and 26820.

TABLE VII
Selected ID3 duplexes

| | SEQ | | SEQ | | |
|---|---|---|---|---|---|
| dsRNA | ID | Sense strand | ID | AntiSense strand | |
| Name | NO: | (5′ > 3′) | NO: | (5′ > 3′) | Type |
| ID3_25 | 26833 | AGCUUAGCCAGGUGGAAAA | 26842 | UUUUCCACCUGGCUAAGCU | 18 + 1 |
| ID3_26 | 26834 | AGCUCACUCCGGAACUUGA | 26843 | UCAAGUUCCGGAGUGAGCU | 18 + 1 |
| ID3_27 | 26835 | CGACAUGAACCACUGCUAA | 26844 | UUAGCAGUGGUUCAUGUCG | 18 + 1 |
| ID3_28 | 26836 | GCUCACUCCGGAACUUGUA | 26845 | UACAAGUUCCGGAGUGAGC | 18 + 1 |
| ID3_29 | 26837 | CGAGCUCACUCCGGAACUA | 26846 | UAGUUCCGGAGUGAGCUCG | 18 + 1 |
| ID3_32 | 26838 | UGUGCUGCCUGUCGGAACA | 26847 | UGUUCCGACAGGCAGCACA | 18 + 1 |
| ID3_34 | 26839 | GAACGCAGGUGCUGGCGCA | 26848 | UGCGCCAGCACCUGCGUUC | 18 + 1 |
| ID3_35 | 26840 | GCUGCUACGAGGCGGUGUA | 26849 | UACACCGCCUCGUAGCAGC | 18 + 1 |
| ID3_37 | 26841 | CCCGGUGCGCGGCUGCUAA | 26850 | UUAGCAGCCGCGCACCGGG | 18 + 1 |
| ID3_9 | 26851 | AGCUUAGCCAGGUGGAAAU | 26859 | AUUUCCACCUGGCUAAGCU | 19 |
| ID3_10 | 26852 | ACGACAUGAACCACUGCUA | 26860 | UAGCAGUGGUUCAUGUC GU | 19 |
| ID3_11 | 26853 | GACAUGAACCACUGCUACU | 26861 | AGUAGCAGUGGUUCAUGUC | 19 |
| ID3_30 | 26854 | AGCUCACUCCGGAACUUGU | 26862 | ACAAGUUCCGGAGUGAGCU | 19 |
| ID3_31 | 26855 | CGAGCUCACUCCGGAACUU | 26863 | AAGUUCCGGAGUGAGCUCG | 19 |
| ID3_33 | 26856 | CCGCCUGCGGGAACUGGUA | 26864 | UACCAGUUCCCGCAGGCGG | 19 |
| ID3_36 | 26857 | GAGGCACUCAGCUUAGCCA | 26865 | UGGCUAAGCUGAGUGCCUC | 19 |
| ID3_38 | 26858 | CGACAUGAACCACUGCUAC | 26866 | GUAGCAGUGGUUCAUGUCG | 19 |

In some embodiments the dsRNA includes an antisense strand and a sense strand having the ID3_{—}9 antisense and sense sequences set forth in SEQ ID NOS:26859 and 26851. In some embodiments the dsRNA includes an antisense strand and a sense strand having the ID3_{—}10 antisense and sense sequences set forth in SEQ ID NOS:26860 and 26852. In some embodiments the dsRNA includes an antisense strand and a sense strand having the ID3_{—}11 antisense and sense sequences set forth in SEQ ID NOS:26861 and 26853. In some embodiments the dsRNA includes an antisense strand and a sense strand having the ID3_{—}25 antisense and sense sequences set forth in SEQ ID NOS:26842 and 26833. In some embodiments the dsRNA includes an antisense strand and a sense strand having the ID3_{—}26 antisense and sense sequences set forth in SEQ ID NOS:26843 and 26834. In some embodiments the dsRNA includes an antisense strand and a sense strand having the ID3_{—}27 antisense and sense sequences set forth in SEQ ID NOS:26844 and 26835. In some embodiments the dsRNA includes an antisense strand and a sense strand having the ID3_{—}28 antisense and sense sequences set forth in SEQ ID NOS:26845 and 26836. In some embodiments the dsRNA includes an antisense strand and a sense strand having the ID3_{—}29 antisense and sense sequences set forth in SEQ ID NOS:26846 and 26837. In some embodiments the dsRNA includes an antisense strand and a sense strand having the ID3_{—}30 antisense and sense sequences set forth in SEQ ID NOS:26862 and 26854. In some embodiments the dsRNA includes an antisense strand and a sense strand having the ID3_{—}31 antisense and sense sequences set forth in SEQ ID NOS:26863 and 26855. In some embodiments the dsRNA includes an antisense strand and a sense strand having the ID3_{—}32 antisense and sense sequences set forth in SEQ ID NOS:26847 and 26838. In some embodiments the dsRNA includes an antisense strand and a sense strand having the ID3_{—}33 antisense and sense sequences set forth in SEQ ID NOS:26864 and 26856. In some embodiments the dsRNA includes an antisense strand and a sense strand having the ID3_{—}34 antisense and sense sequences set forth in SEQ ID NOS:26848 and 26839. In some embodiments the dsRNA includes an antisense strand and a sense strand having the ID3_{—}35 antisense and sense sequences set forth in SEQ ID NOS:26849 and 26840. In some embodiments the dsRNA includes an antisense strand and a sense strand having the ID3_{—}36 antisense and sense sequences set forth in SEQ ID NOS:26865 and 26857. In some embodiments the dsRNA includes an antisense strand and a sense strand having the ID3_{—}37 antisense and sense sequences set forth in SEQ ID NOS:26850 and 26841. In some embodiments the dsRNA includes an antisense strand and a sense strand having the ID3_{—}38 antisense and sense sequences set forth in SEQ ID NOS:26866 and 26858.

In some preferred embodiments the dsRNA includes an antisense strand and a sense strand having the ID3_{—}32 antisense and sense sequences (SEQ ID NOS:26847 and 26838). In some preferred embodiments the dsRNA includes an antisense strand and a sense strand having the ID3_{—}38 antisense and sense sequences (SEQ ID NOS:26866 and 26858).

In some embodiments the dsRNA includes an antisense strand and a sense strand having the ID3_{—}32 antisense and sense sequences set forth in SEQ ID NOS:26847 and 26838 and includes the following modifications:

The sense strand includes a 2′OMe sugar modified ribonucleotide in position 1, a capping moiety covalently attached at the 5 terminus, a non-nucleotide moiety covalently attached at the 3′ terminus and 5 consecutive 2′5′ ribonucleotides in positions 15, 16, 17, 18, and 19; and the antisense strand includes 2′OMe sugar modified ribonucleotides in positions 2, 5, 8, 13, 15 and 18, and optionally includes a 2′5′ ribonucleotide in position 5, 6 or 7 and a non-nucleotide moiety covalently attached to the 3′ terminus. In some embodiments the antisense strand is phosphorylated at its 5′ terminus. In preferred embodiments the capping moiety is an inverted abasic deoxyribonucleotide moiety. In preferred embodiments the non-nucleotide moiety covalently attached at the 3′ end of the sense strand is a C3Pi moiety. In preferred embodiments the non-nucleotide moiety covalently attached at the 3′ end of the antisense strand is a C3PiC3OH or C3PiC3Pi moiety.

In some embodiments the dsRNA includes an antisense strand and a sense strand having the ID3_{—}38 antisense and sense sequences set forth in SEQ ID NOS:26866 and 26858 and includes the following modifications:

The sense strand includes 2′OMe sugar modified ribonucleotide in positions 1 and 3, a capping moiety covalently attached at the 5 terminus, a non-nucleotide moiety covalently attached at the 3′ terminus and 5 consecutive 2′5′ ribonucleotides in positions 15, 16, 17, 18, and 19; and the antisense strand includes 2′OMe sugar modified ribonucleotides in positions 1, 6, 9, 13, 16 and 18, and a non-nucleotide moiety covalently attached to the 3′ terminus. In some embodiments the antisense strand is phosphorylated at its 5′ terminus. In preferred embodiments the capping moiety is an inverted abasic deoxyribonucleotide moiety. In preferred embodiments the non-nucleotide moiety covalently attached at the 3′ end of the sense strand is a C3Pi moiety. In preferred embodiments the non-nucleotide moiety covalently attached at the 3′ end of the antisense strand is a C3PiC3OH or C3PiC3Pi moiety.

TABLE VIII
Selected CDKN1B (p27) duplexes

| | SEQ | | | | |
|---|---|---|---|---|---|
| | ID | Sense strand | SEQ ID | AntiSense strand | |
| DsRNA Name | NO: | (5′ > 3′) | NO: | (5′ > 3′) | Type |
| CDKN1B_29 | 26867 | AGCCAAAGUGGCAUGUUUA | 26877 | UAAACAUGCCACUUUGGCU | 18 + 1 |
| CDKN1B_30 | 26868 | GCAUACUGAGCCAAGUAUA | 26878 | UACAUCCUGGCUCUCCUGC | 18 + 1 |
| CDKN1B_31 | 26869 | CAGCGCAAGUGGAAUUUCA | 26879 | UGAAAUUCCACUUGCGCUG | 18 + 1 |
| CDKN1B_33 | 26870 | UGCAUACUGAGCCAAGUAA | 26880 | UAUGCCACUUUGGCUUGUA | 18 + 1 |
| CDKN1B_34 | 26871 | GGAGCGGAUGGACGCCAGA | 26881 | UCUGACAUCCUGGCUCUCC | 18 + 1 |
| CDKN1B_35 | 26872 | AGGGCAGCUUGCCCGAGUA | 26882 | UACUCGGGCAAGCUGCCCU | 18 + 1 |
| CDKN1B_36 | 26873 | GUACUACCUGUGUAUAUAG | 26883 | UUUGGCUCAGUAUGCAACC | 18 + 1 |
| CDKN1B_37 | 26874 | UGCAUACUGAGCCAAGUAU | 26884 | UUACUUGGCUCAGUAUGCA | 18 + 1 |
| CDKN1B_38 | 26875 | GAGUGUCUAACGGGAGCCA | 26885 | UCCGCUGACAUCCUGGCUC | 18 + 1 |
| CDKN1B_40 | 26876 | GCGCAAGUGGAAUUUCGAA | 26886 | UUCGAAAUUCCACUUGCGC | 18 + 1 |
| CDKN1B_3 | 26887 | CGCAUUUGGUGGACCCAAA | 26894 | UUUGGGUCCACCAAAUGCG | 19 |
| CDKN1B_4 | 26888 | GCAAUUAGGUUUUUCCUUA | 26895 | UAAGGAAAAACCUAAUUGC | 19 |
| CDKN1B_10 | 26889 | CAUUGUACUACCUGUGUAU | 26896 | AUACACAGGUAGUACAAUG | 19 |
| CDKN1B_11 | 26890 | GGUUUUUCCUUAUUUGCUU | 26897 | AAGCAAAUAAGGAAAAACC | 19 |
| CDKN1B_18 | 26891 | AGCGCAAGUGGAAUUUCGA | 26898 | UCGAAAUUCCACUUGCGCU | 19 |
| CDKN1B_28 | 26892 | GGUUGCAUACUGAGCCAAA | 26899 | AUCCUGGCUCUCCUGCGCC | 19 |
| CDKN1B_32 | 26893 | AGCCAAAGUGGCAUGUUUU | 26900 | AAAACAUGCCACUUUGGCU | 19 |

In some embodiments the dsRNA includes an antisense strand and a sense strand having the CDKN1B_{—}3 antisense and sense sequences set forth in SEQ ID NOS:26894 and 26887. In some embodiments the dsRNA includes an antisense strand and a sense strand having the CDKN1B_{—}4 antisense and sense sequences set forth in SEQ ID NOS:26895 and 26888. In some embodiments the dsRNA includes an antisense strand and a sense strand having the CDKN1B_{—}10 antisense and sense sequences set forth in SEQ ID NOS:26896 and 26889. In some embodiments the dsRNA includes an antisense strand and a sense strand having the CDKN1B_{—}11 antisense and sense sequences set forth in SEQ ID NOS:26897 and 26890. In some embodiments the dsRNA includes an antisense strand and a sense strand having the CDKN1B_{—}18 antisense and sense sequences set forth in SEQ ID NOS:26898 and 26891. In some embodiments the dsRNA includes an antisense strand and a sense strand having the CDKN1B_{—}28 antisense and sense sequences set forth in SEQ ID NOS:26866 and 26858. In some embodiments the dsRNA includes an antisense strand and a sense strand having the CDKN1B_{—}29 antisense and sense sequences set forth in SEQ ID NOS:26899 and 26892. In some embodiments the dsRNA includes an antisense strand and a sense strand having the CDKN1B_{—}30 antisense and sense sequences set forth in SEQ ID NOS:26878 and 26868. In some embodiments the dsRNA includes an antisense strand and a sense strand having the CDKN1B_{—}31 antisense and sense sequences set forth in SEQ ID NOS:26879 and 26869. In some embodiments the dsRNA includes an antisense strand and a sense strand having the CDKN1B_{—}32 antisense and sense sequences set forth in SEQ ID NOS:26900 and 26893. In some embodiments the dsRNA includes an antisense strand and a sense strand having the CDKN1B_{—}33 antisense and sense sequences set forth in SEQ ID NOS:26880 and 26870. In some embodiments the dsRNA includes an antisense strand and a sense strand having the CDKN1B_{—}34 antisense and sense sequences set forth in SEQ ID NOS:268881 and 26871. In some embodiments the dsRNA includes an antisense strand and a sense strand having the CDKN1B_{—}35 antisense and sense sequences set forth in SEQ ID NOS:26882 and 26872. In some embodiments the dsRNA includes an antisense strand and a sense strand having the CDKN1B_{—}36 antisense and sense sequences set forth in SEQ ID NOS:26883 and 26873. In some embodiments the dsRNA includes an antisense strand and a sense strand having the CDKN1B_{—}37 antisense and sense sequences set forth in SEQ ID NOS:26884 and 26874. In some embodiments the dsRNA includes an antisense strand and a sense strand having the CDKN1B_{—}38 antisense and sense sequences set forth in SEQ ID NOS:26885 and 26875. In some embodiments the dsRNA includes an antisense strand and a sense strand having the CDKN1B_{—}40 antisense and sense sequences set forth in SEQ ID NOS:26886 and 26876.

In some preferred embodiments the dsRNA includes an antisense strand and a sense strand having the CDKN1B_{—}4 antisense and sense sequences set forth in SEQ ID NOS:26895 and 26888. In some preferred embodiments the dsRNA includes an antisense strand and a sense strand having the CDKN1B_{—}31 antisense and sense sequences set forth in SEQ ID NOS:26879 and 268698. In some preferred embodiments the dsRNA includes an antisense strand and a sense strand having the CDKN1B_{—}40 antisense and sense sequences set forth in SEQ ID NOS:26886 and 26876.

TABLE IX
Selected NOTCH1 dsRNA

| | SEQ | | SEQ | | |
|---|---|---|---|---|---|
| | ID | | ID | Antisense strand | |
| Name | NO: | Sense strand 5′>3′ | NO: | 5′>3′ | |
| NOTCH_1 | 26901 | CCUUCUACUGCGAGUGUCA | 26906 | UGACACUCGCAGUAGAAGG | 18 + 1 |
| NOTCH1_2 | 26902 | GCUACAACUGCGUGUGUGA | 26907 | UCACACACGCAGUUGUAGC | 18 + 1 |
| NOTCH1_3 | 26903 | UCCUUCUACUGCGAGUGUA | 26908 | UACACUCGCAGUAGAAGGA | 18 + 1 |
| NOTCH1_4 | 26904 | CUCCUUCUACUGCGAGUGA | 26909 | UCACUCGCAGUAGAAGGAG | 18 + 1 |
| NOTCH1_5 | 26905 | CAGCGCAGAUGCCAACAUA | 26910 | UAUGUUGGCAUCUGCGCUG | 18 + 1 |
| NOTCH1_6 | 26911 | ACAACUGCGUGUGUGUCAA | 26912 | UUGACACACACGCAGUUGU | 19 |

In some embodiments the dsRNA includes an antisense strand and a sense strand having the NOTCH1_{—}1 antisense and sense sequences set forth in SEQ ID NOS:26906 and 26901. In some embodiments the dsRNA includes an antisense strand and a sense strand having the NOTCH1_{—}2 antisense and sense sequences set forth in SEQ ID NOS:26907 and 26902. In some embodiments the dsRNA includes an antisense strand and a sense strand having the NOTCH1_{—}3 antisense and sense sequences set forth in SEQ ID NOS:26908 and 26903. In some embodiments the dsRNA includes an antisense strand and a sense strand having the NOTCH1_{—}4 antisense and sense sequences set forth in SEQ ID NOS:26909 and 26904. In some embodiments the dsRNA includes an antisense strand and a sense strand having the NOTCH1_{—}5 antisense and sense sequences set forth in SEQ ID NOS:26910 and 26905. In some embodiments the dsRNA includes an antisense strand and a sense strand having the NOTCH1_{—}6 antisense and sense sequences set forth in SEQ ID NOS:26912 and 26911.

In some preferred embodiments the dsRNA includes an antisense strand and a sense strand having the NOTCH1_{—}2 antisense and sense sequences set forth in SEQ ID NOS:26907 and 26902.

In some embodiments provided herein is a double stranded RNA molecule which includes a sense strand and an antisense strand selected from the oligonucleotide pairs set forth in Tables I-IX. Unless otherwise stated all positions along a sense strand or antisense strand are counted from the 5′ to the 3′ (5′-3′).

In some embodiments a double stranded nucleic acid molecule includes a particular sense strand and a particular antisense strand set forth in SEQ ID NOS:23-26,666, preferably shown in Tables I-IX (SEQ ID NOS:26,667-26,912). In some embodiments the double stranded nucleic acid molecule has the structure:

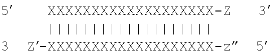

wherein each “|” represents base pairing between the ribonucleotides;

wherein each X is any one of A, C, G, U and is independently an unmodified or modified ribonucleotide, an unmodified or modified deoxyribonucleotide or an unconventional moiety;

wherein each of Z and Z′ is independently present or absent, but if present is independently 1-5 consecutive nucleotides or non-nucleotide moieties or a combination thereof covalently attached at the 3′ terminus of the strand in which it is present; and

wherein z″ may be present or absent, but if present is a capping moiety covalently attached at the 5′ terminus of the sense strand.

In preferred embodiments the double-stranded nucleic acid molecule comprises modified ribonucleotides and unconventional moieties.

In some embodiments provided is a double-stranded nucleic acid molecule wherein the antisense strand includes a mirror nucleotide or a 2′-5′ linked ribonucleotide in one or more of positions 5, 6, 7 or 8 (5′-3′), and a nucleotide or non-nucleotide moiety covalently attached at the 3′ terminus. In some embodiments the antisense strand further includes one or more 2′OMe sugar modified ribonucleotides. In some embodiments 1, 2, 3, 4, 5, 6 or 7 pyrimidine ribonucleotides in the antisense strand are 2′OMe sugar modified pyrimidine ribonucleotides. In some embodiments the sense strand includes 4 or 5 consecutive 2′-5′ linked nucleotides at the 3′ terminal or penultimate positions, a nucleotide or non-nucleotide moiety covalently attached at the 3′ terminus, one or more 2′OMe sugar modified ribonucleotides, and a capping moiety covalently attached at the 5′ terminus. The dsRNA molecule may include a 5′ phosphate on the antisense strand.

In some embodiments provided is a double-stranded nucleic acid molecule wherein the antisense strand includes (5′>3′) 2′OMe sugar modified ribonucleotides at positions 1, 3, 11, 14, 15, 17 and 18, and a C3Pi-C3OH moiety covalently attached to the 3′ terminus; and the sense strand includes (5′>3′) 2′-5′ linked ribonucleotides at positions 15, 16, 17, 18 and 19, a 3′ terminal nucleotide or non-nucleotide overhang; and a capping moiety covalently attached at the 5′ terminus. In some embodiments the antisense strand further includes a 2′-5′ linked ribonucleotide at position 6, at position 7 or at positions 6 and 7.

In some embodiments provided is a double-stranded nucleic acid molecule wherein the antisense strand includes (5′>3′) 2′OMe sugar modified ribonucleotides at positions 1, 3, 6, 11, 14, 15, 17 and 18, and a C3Pi-C3OH moiety covalently attached to the 3′ terminus; and the sense strand includes (5′>3′) 2′-5′ linked ribonucleotides at positions 15, 16, 17, 18 and 19, a C3Pi or C3OH moiety covalently attached to the 3′ terminus; and a capping moiety covalently attached at the 5′ terminus.

In some embodiments provided is a double-stranded nucleic acid molecule wherein the antisense strand includes (5′>3′) 2′OMe sugar modified ribonucleotides at positions 1, 3, 6, 11, 14, 15, 17 and 18, and a C3Pi-C3OH moiety covalently attached to the 3′ terminus; and the sense strand includes (5′>3′) 2′-5′ linked ribonucleotides at positions 15, 16, 17, 18 and 19, a C3Pi covalently attached to the 3 terminus and an inverted abasic deoxyribonucleotide capping moiety covalently attached at the 5′ terminus, In some embodiments provided is a double stranded nucleic acid molecule wherein the antisense strand includes (5′>3′) 2′OMe sugar modified ribonucleotides at positions 1, 3, 11, 14, 15, 17 and 18, a 2′-5′ linked ribonucleotide or a mirror nucleotide in one or more of positions 6, 7 and 8, and a C3Pi-C3OH moiety covalently attached to the 3′ terminus; and the sense strand includes (5′>3′) 2′-5′ linked ribonucleotides at positions 15, 16, 17, 18 and 19, a 3′ terminal nucleotide or non-nucleotide overhang; and a capping moiety covalently attached at the 5′ terminus.

In some embodiments provided is a double-stranded nucleic acid molecule wherein the antisense strand includes (5′>3′) 2′OMe sugar modified ribonucleotides at positions 1, 3, 11, 14, 15, 17 and 18, a 2′-5′ linked ribonucleotide at position 6, and a C3Pi-C3OH moiety covalently attached to the 3′ terminus; and the sense strand includes (5′>3′) 2′-5′ linked ribonucleotides at positions 15, 16, 17, 18 and 19, a C3Pi or C3OH moiety covalently attached to the 3′ terminus; and a capping moiety selected from an abasic moiety, an inverted abasic moiety, a C6 amino and a mirror nucleotide covalently attached at the 5′ terminus.

In some embodiments provided is a double-stranded nucleic acid molecule wherein the antisense strand includes (5′>3′) 2′OMe sugar modified ribonucleotides at positions 1, 3, 11, 14, 15, 17 and 18, a 2′-5′ linked ribonucleotide at position 6, and a C3Pi-C3OH moiety covalently attached to the 3′ terminus; and the sense strand includes (5′>3′) 2′-5′ linked ribonucleotides at positions 15, 16, 17, 18 and 19, a C3Pi covalently attached to the 3 terminus and an inverted abasic deoxyribonucleotide capping moiety covalently attached at the 5′ terminus.

In some embodiments provided is a double-stranded nucleic acid molecule wherein the antisense strand includes (5′>3′) 2′OMe sugar modified ribonucleotides at positions 1, 3, 11, 14, 15, 17 and 18, a 2′-5′ linked ribonucleotide at position 6, and a C3Pi-C3OH moiety covalently attached to the 3′ terminus; and the sense strand includes (5′>3′) 2′-5′ linked ribonucleotides at positions 15, 16, 17, 18 and 19, a C3Pi covalently attached to the 3 terminus and a mirror nucleotide (L-deoxyriboguanosine-3′-phosphate) covalently attached at the 5′ terminus.

In some embodiments provided is a double-stranded nucleic acid molecule wherein the antisense strand includes (5′>3′) 2′OMe sugar modified ribonucleotides at positions 1, 3, 6, 11, 14, 15, 17 and 18, a 2′-5′ linked ribonucleotide at position 7, and a C3Pi-C3OH moiety covalently attached to the 3′ terminus; and the sense strand includes (5′>3′) an optional 2′OMe sugar modified ribonucleotide at position 1,2′-5′ linked ribonucleotides at positions 15, 16, 17, 18 and 19, a C3Pi or C3OH moiety covalently attached to the 3′ terminus; and a capping moiety selected from an abasic moiety, an inverted abasic moiety, a C6 amino and a mirror nucleotide covalently attached at the 5′ terminus.

In some embodiments provided is a double-stranded nucleic acid molecule wherein the antisense strand includes (5′>3′) 2′OMe sugar modified ribonucleotides at positions 1, 3, 6, 11, 14, 15, 17 and 18, a 2′-5′ linked ribonucleotide at position 7, and a C3Pi-C3OH moiety covalently attached to the 3′ terminus; and the sense strand includes (5′>3′) a 2′OMe sugar modified ribonucleotide at position 1, a C3Pi moiety covalently attached to the 3 terminus and an inverted abasic deoxyribonucleotide capping moiety covalently attached at the 5′ terminus;.

In some embodiments provided is a double-stranded nucleic acid molecule wherein the antisense strand includes (5′>3′) 2′OMe sugar modified ribonucleotides at positions 1, 3, 6, 11, 14, 15, 17 and 18, a 2′-5′ linked ribonucleotide at position 7, and a C3Pi-C3OH moiety covalently attached to the 3′ terminus; and the sense strand includes (5′>3′) a 2′OMe sugar modified ribonucleotide at position 1, and 2′-5′ linked ribonucleotides at positions 15, 16, 17, 18 and 19, a C3Pi covalently attached to the 3 terminus and a mirror nucleotide (L-deoxyriboguanosine-3′-phosphate) covalently attached at the 5′ terminus.

In some embodiments provided is a double stranded nucleic acid molecule wherein the antisense strand includes (5′>3′) 2′OMe sugar modified ribonucleotides at positions 1, 3, 11, 14, 15, 17 and 18, 2′-5′ linked ribonucleotide at positions 6 and 7 and a C3Pi-C3OH moiety covalently attached to the 3′ terminus; and the sense strand includes (5′>3′) an optional 2′OMe sugar modified ribonucleotide at position 1,2′-5′ linked ribonucleotides at positions 15, 16, 17, 18 and 19, a C3Pi or C3OH moiety covalently attached to the 3′ terminus and a capping moiety selected from an abasic moiety, an inverted abasic moiety and a mirror nucleotide covalently attached at the 5′ terminus.

In some embodiments provided is a double-stranded nucleic acid molecule wherein the antisense strand includes (5′>3′) 2′OMe sugar modified ribonucleotides at positions 1, 3, 11, 14, 15, 17 and 18, a 2′-5′ linked ribonucleotide at positions 6 and 7 and a C3Pi-C3OH moiety covalently attached to the 3′ terminus; and the sense strand includes (5′>3′) a 2′OMe sugar modified ribonucleotide at position 1,2′-5′ linked ribonucleotides at positions 15, 16, 17, 18 and 19, a C3Pi moiety covalently attached to the 3 terminus and an inverted abasic deoxyribonucleotide capping moiety covalently attached at the 5′ terminus,

In some embodiments provided is a double-stranded nucleic acid molecule wherein the antisense strand includes (5′>3′) 2′OMe sugar modified ribonucleotides at positions 1, 3, 11, 14, 15, 17 and 18, a mirror nucleotide at position 6 and a C3Pi-C3OH moiety covalently attached to the 3′ terminus; and the sense strand includes (5′>3′) a 2′OMe sugar modified ribonucleotide at position 1, and 2′-5′ linked ribonucleotides at positions 15, 16, 17, 18 and 19, a C3Pi moiety covalently attached to the 3 terminus and an inverted abasic deoxyribonucleotide capping moiety covalently attached at the 5′ terminus In some embodiments provided is a double stranded nucleic acid molecule wherein the antisense strand includes (5′>3′) a 2′OMe sugar modified ribonucleotides at positions 1, 3, 11, 14, 15, 17 and 18, a mirror nucleotide at position 8 and a C3Pi-C3OH moiety covalently attached to the 3′ terminus; and the sense strand includes (5′>3′) a 2′OMe sugar modified ribonucleotide at position 1,2′-5′ linked ribonucleotides at positions 15, 16, 17, 18 and 19, a C3Pi moiety covalently attached to the 3 terminus and an inverted abasic deoxyribonucleotide capping moiety covalently attached at the 5′ terminus.

In some embodiments provided is a double-stranded nucleic acid molecule wherein the sense strand includes (5′>3′) a 2′OMe sugar modified ribonucleotide at position 1,2′-5′ linked ribonucleotides at positions 15, 16, 17, 18 and 19, a C3Pi moiety covalently attached to the 3 terminus and an inverted abasic deoxyribonucleotide cap moiety covalently attached at the 5′ terminus, and the antisense strand is selected from
an antisense oligonucleotide which includes (5′>3′) a U to dT substitution in position 1, a 5′ phosphate covalently attached to the deoxyribothymidine in position 1, 2′OMe sugar modified ribonucleotides at positions 3, 11, 14, 15, 17 and 18, a 2′-5′ linked ribonucleotide at position 7 and a C3Pi-C3OH moiety covalently attached to the 3′ terminus; or
an antisense oligonucleotide which includes (5′>3′) a 5′ phosphate covalently attached to the uridine in position 1, 2′OMe sugar modified ribonucleotides at positions 3, 11, 14, 15, 17 and 18, a 2′-5′ linked ribonucleotide at position 7 and a C3Pi-C3OH moiety covalently attached to the 3′ terminus; or an antisense oligonucleotide which includes (5′>3′) a U to C3 substitution in position 1, a 5′ phosphate covalently attached to the C3 in position 1, 2′OMe sugar modified ribonucleotides at positions 3, 11, 14, 15, 17 and 18, a 2′-5′ linked ribonucleotide at position 7 and a C3Pi-C3OH moiety covalently attached to the 3′ terminus; or
an antisense oligonucleotide which includes (5′>3′) a 5′ phosphate covalently attached to the uridine in position 1, 2′OMe sugar modified ribonucleotides at positions 1, 3, 11, 14, 15, 17 and 18, a 2′-5′ linked ribonucleotide at position 7 and a C3Pi-C3OH moiety covalently attached to the 3′ terminus.

The above modifications can be applied to the nucleic acid pairs (sense and corresponding antisense oligonucleotides) set forth in SEQ ID NOS:23-26,666 and, more particularly, to the dsRNA listed in Tables I-IX (SEQ ID NOS:26667-26,912).

Certain preferred duplexes are set forth herein below in Table A

TABLE A

| Name | Sense 5 −> 3 | Antisense 5 −> 3 |
|---|---|---|
| HES1_12_S1938 | zidB; rG; rC; mC; rA; rG; rC; mU; | p; mU; rU; rC; mC; rA; rU; rU2p; rA; |
| | rG; rA; mU; rA; mU; rA; rA; mU; rG; | mU; rA; rU; mC; rA; rG; rC; mU; rG; |
| | rG; rA; rA; zc3p | rG; mC; zc3p; zc3p |
| HES1_12_S1939 | zidB; rG; rC; mC; rA; rG; rC; mU; rG; | p; mU; rU; rC; mC; rA; rU; rU2p; rA; |
| | rA; mU; rA; mU; rA; rA; rU2p; rG2p; | mU; rA; rU; mC; rA; rG; rC; mU; rG; |
| | rG2p; rA2p; rA2p; zc3p | rG; mC; zc3p; zc3p |
| HES1_13_S1940 | zidB; rG; rC; mC; rA; rG; mU; rG; rU; | p; mU; rU; mG; rU; mC; rG; rU2p; rG; |
| | mC; rA; rA; mC; rA; rC; rG2p; rA2p; | mU; rU; mG; rA; mC; rA; rC; mU; rG; |
| | rC2p; rA2p; rA2p; zc3p | rG; mC; zc3p; zc3p |
| HES1_13_S1941 | zidB; rG; rC; mC; rA; rG; mU; rG; rU; | p; mU; rU; mG; rU; mC; rG; rU2p; rG; |
| | mC; rA; rA; mC; rA; rC; rG; rA; mC; | mU; rU; mG; rA; mC; rA; rC; mU; rG; |
| | rA; rA; zc3p | rG; mC; zc3p; zc3p |
| HES1_13_S1942 | zidB; rG; rC; mC; rA; rG; mU; rG; rU; | p; rU; mU; rG; rU; mC; rG; rU2p; rG; |
| | mC; rA; rA; mC; rA; rC; rG2p; rA2p; | rU; mU; rG; rA; mC; rA; rC; mU; rG; |
| | rC2p; rA2p; rA2p; zc3p | rG; mC; zc3p; zc3p |
| HES1_13_S1943 | zidB; rG; rC; mC; rA; rG; mU; rG; rU; | p; rU; mU; rG; rU; mC; rG; rU2p; rG; |
| | mC; rA; rA; mC; rA; rC; rG; rA; mC; | rU; mU; rG; rA; mC; rA; rC; mU; rG; |
| | rA; rA; zc3p | rG; mC; zc3p; zc3p |
| HEY2_1_S1944 | zidB; rG; rG; rG; rA; rG; mC; rG; rA; | p; mU; rG; mU; rA; rA; rU; rU2p; rG; |
| | rG; rA; rA; mC; rA; rA; rU2p; rU2p; | mU; rU; mC; rU; mC; rG; mC; rU; mC; |
| | rA2p; rC2p; rA2p; zc3p | rC; mC; zc3p; zc3p |
| HEY2_1_S1945 | zidB; rG; rG; rG; rA; rG; mC; rG; rA; | p; mU; rG; mU; rA; rA; rU; rU2p; rG; |
| | rG; rA; rA; mC; rA; rA; rU; mU; rA; | mU; rU; mC; rU; mC; rG; mC; rU; mC; |
| | mC; rA; zc3p | rC; mC; zc3p; zc3p |
| HEY2_2_S1946 | zidB; rG; rG; rG; mU; rA; rA; rA; rG; | p; mU; rU; mC; rA; rA; rA; rG2p; mU; |
| | rG; rC; mU; rA; mC; rU; rU2p; rU2p; | rA; rG; mC; rC; mU; rU; mU; rA; mC; |
| | rG2p; rA2p; rA2p; zc3p | rC; mC; zc3p; zc3p |
| HEY2_2_S1947 | zidB; rG; rG; rG; mU; rA; rA; rA; rG; | p; mU; rU; mC; rA; rA; rA; rG2p; mU; |
| | rG; rC; mU; rA; mC; rU; rU; mU; rG; | rA; rG; mC; rC; mU; rU; mU; rA; mC; |
| | rA; rA; zc3p | rC; mC; zc3p; zc3p |

For example, HES1_{—}12_S1938, is a duplex which includes a sense strand with an oligonucleotide sequence 5′>3′ GCCAGCUGAUAUAAUGGAA in which the ribonucleotides at positions 1, 2, 4, 5, 6, 8, 9, 11, 13, 14, 16, 17, 18, and 19 are unmodified ribonucleotides, the ribonucleotides at positions 3, 7, 10, 12 and 15 are 2′OMe sugar modified ribonucleotides, an inverted abasic moiety is covalently attached to the 5′ terminus and a C3-Pi moiety is covalently attached to the 3′ terminus; and an antisense strand with an oligonucleotide sequence 5′>3′ UUCCAUUAUAUCAGCUGGC in which the ribonucleotides at positions 2, 3, 5, 6, 8, 10, 11, 13, 14, 15, 17 and 18 are unmodified ribonucleotides, the ribonucleotides at positions 1, 4, 9, 12, 16 and 19 are 2′OMe sugar modified ribonucleotides, the ribonucleotide at position 7 is a 2′-5′ linked ribonucleotide, a phosphate is optionally covalently attached to the 5′ terminus and a C3Pi-C3Pi moiety is covalently attached to the 3′ terminus.

In a second aspect provided are compositions comprising one or more such nucleic acid compounds disclosed herein; and a pharmaceutically acceptable carrier or excipient. In some embodiments the dsRNA molecule is administered as naked dsRNA. In other embodiments the dsRNA molecule is admixed with a pharmaceutically acceptable carrier. In yet other embodiments the dsRNA is encapsulated in a drug carrier.

In a third aspect provided is use of the molecules disclosed herein in treating a subject suffering from disease or disorder of the ear. Provided herein are methods for treating or preventing the incidence or severity of hearing loss in a subject in need thereof wherein the hearing loss is associated with expression of a target gene selected from a gene set forth in Table 1A, set forth in any one of SEQ ID NOS:1-11. Such methods involve administering to a mammal in need of such treatment a prophylactically or therapeutically effective amount of one or more such compounds, which inhibit or reduce expression or activity of at least one such target gene. In various embodiments, the method disclosed herein provides for inhibiting more than one target gene associated with the ear disorder using one or more dsRNA molecule disclosed herein.

Accordingly, in one embodiment, the method is directed to treating a subject at risk of acquiring, or suffering from, an ear disorder by inhibiting two or more target genes at least one of CDKN1B and ID3, optionally in combination with at least one of HES1, HES5, HEY1 or HEY2.

In a preferred embodiment, the method comprises administering the oligonucleotides directed against at least one of CDKN1B and ID3 prior to administering the oligonucleotides directed against at least one of HES1, HES5. In another embodiment, all oligonucleotides are administered together.

In some embodiments dsRNA to HES1 and HES5 are co-administered.

In some embodiments dsRNA to HES1 and dsRNA to HES5 and optionally HEY2 are co-administered. In some embodiments dsRNA targeting CDKN1B is administered followed by dsRNA to HES1, or dsRNA to HES5, or dsRNA to HES1 and dsRNA to HES5, or dsRNA to HES1 and dsRNA to HEY1, or dsRNA to HES5 and dsRNA to HEY1, or dsRNA to HES1 and dsRNA to HEY2, or dsRNA to HES5 and dsRNA to HEY2; optionally followed by dsRNA to ID1 or dsRNA to ID2, or dsRNA to ID3.

In some embodiments administration of dsRNA targeting HES1 or HES5, is followed by dsRNA to HEY1 or dsRNA to HEY2; optionally followed by dsRNA to ID1 or dsRNA to ID2, or dsRNA to ID3. In some embodiments administration of dsRNA targeting NOTCH1, is followed by dsRNA to HEY1 or dsRNA to HEY2; optionally followed by dsRNA to ID1 or dsRNA to ID2, or dsRNA to ID3.

In some embodiments the at least two dsRNA agents are co-administered, e.g. concomitantly or in sequence. In other embodiments, the at least two dsRNA agents are administered in a pharmaceutical composition comprising a combination thereof. In some embodiments the dsRNA agent is a combined inhibitor by which it is meant a single agent which is capable of down-regulating the expression and/or activity of at least two genes and/or gene products selected from the group consisting of HEST, HES5, HEY1, HEY2, ID1, ID2, ID3, CDKN1B, and NOTCH1. Non-limiting examples of such single agents are tandem and multi-armed RNAi molecules disclosed in PCT Patent Publication No. WO 2007/091269.

In another aspect provided is use of a nucleic acid compound disclosed herein for the preparation of a medicament for the treatment of a disease or disorder of the inner or middle ear.

In particular embodiments, provided herein are chemically modified dsRNA oligonucleotides, compositions comprising same and methods of use thereof in the treatment of auditory and vestibular diseases, disorders, injuries and conditions including, without being limited to ototoxin induced hearing loss, age-related hearing loss, a hearing impairment due to end-organ lesions involving inner ear hair cells, e.g., acoustic trauma, viral endolymphatic labyrinthitis, Meniere's disease; tinnitus which may be intermittent or continuous, wherein there is diagnosed a sensorineural loss; hearing loss due to bacterial or viral infection, such as in herpes zoster oticus; purulent labyrinthitis arising from acute otitis media, purulent meningitis, chronic otitis media, sudden deafness including that of viral origin, e.g., viral endolymphatic labyrinthitis caused by viruses including mumps, measles, influenza, chicken pox, mononucleosis and adenoviruses; congenital hearing loss such as that caused by rubella, anoxia during birth, bleeding into the inner ear due to trauma during delivery, ototoxic drugs administered to the mother, erythroblastosis fetalis, and hereditary conditions including Waardenburg's syndrome and Hurler's syndrome.

Further provided is a method of preventing degeneration of the auditory nerve, (also known as the vestibulocochlear nerve or acoustic nerve) responsible for transmitting sound and equilibrium information from the inner ear to the brain. The hair cells of the inner ear transmit information to the brain via the auditory nerve, which consists of the cochlear nerve, and the vestibular nerve, and emerges from the medulla oblongata and enters the inner skull via the internal acoustic meatus (or internal auditory meatus) in the temporal bone, along with the facial nerve.

Such methods involve administering to a mammal in need of such treatment a prophylactically or therapeutically effective amount of one or more nucleic acid molecules disclosed herein which inhibit or reduce expression or activity of HES1, HES5, HEY1, HEY2, ID1, ID2, ID3, CDKN1B, or NOTCH1.

In another aspect, provided herein is a method for the treatment of a subject in need of treatment for a disease or disorder or symptoms associated with the disease or disorder, associated with the expression of HES1, HES5, HEY1, HEY2, ID1, ID2, ID3, CDKN1B, or NOTCH1, comprising administering to the subject an amount of nucleic acid molecule which reduces or inhibits expression of HES1, HES5, HEY1, HEY2, ID1, ID2, ID3, CDKN1B, or NOTCH1 set forth in SEQ ID NO:1-11.

Additionally provided are novel structures of double stranded oligonucleotides, having advantageous properties and which are applied to dsRNA molecules directed to any target sequence, to inhibit expression of a target gene, particularly the mRNA sequences set forth in SEQ ID NOS:1-11. Provided herein are functional dsRNA nucleic acids comprising various modifications as disclosed herein, their use for the manufacture of a medicament, pharmaceutical compositions comprising such modified functional nucleic acids and methods for the treatment of a patient suffering from or susceptible to disease or disorder as disclosed herein.

In still another embodiment, provided is a method for treating or preventing the incidence or severity of hearing impairment in a patient comprising administering to the patient a composition comprising an effective amount of a naked, chemically synthesized dsRNA compound. Preferably, the naked dsRNA compound is applied directly to the round window membrane of the cochlea or administered by transtympanic injection or via a transtympanic device including a canula.

The preferred methods, materials, and examples that will now be described are illustrative only and are not intended to be limiting; materials and methods similar or equivalent to those described herein can be used in practice or testing of the invention. Other features and advantages of the invention will be apparent from the following figures, detailed description, and from the claims.

### BRIEF DESCRIPTION OF THE FIGURES

FIG. 1: experimental design of a mouse model for aminoglycoside (AG)-induced vestibular sensory epithelium damage in CBA/J mice. Streptomycin is injected into the posterior semi-circular canal (PSC), dsRNA to HES5 was injected into PSC one week later and evaluation is performed two weeks post dsRNA injection. The readouts were as follows: whole-mount staining with Myosin VIIa antibody for counting hair cells (hair cell specific marker); whole-mount staining for Atoh1 positive cells (marker for early developing hair cells); Atoh1 expression (mRNA levels) and HES5 expression (mRNA levels).

FIG. 2A-2B: Cy3 labeled dsRNA is delivered to AG-damaged vestibular sensory epithelium upon local injection into PSC. Cells are labelled with phalloidin. FIG. 2A: PSC, FIG. 2B: utricle. Arrows show Cy3 labeled dsRNA.

FIG. 3A-3B: Treatment with dsRNA to HES5 results in an increase in the amount of hair cells in AG-damaged vestibular epithelia. FIG. 3A: dsRNA treated. FIG. 3B: vehicle treated. Arrows show Myosin VIIa-positive cells, which are identified as transdifferentiated hair cells. Myosin VIIa has been reported to be a hair cell marker (Hasson et al., 1995, PNAS 92:9815-9819).

FIG. 4 shows data that treatment with dsRNA to HES5 facilitates hair cell regeneration.

FIG. 5 shows double anti-Atoh1 and anti-Myosin VIIa staining in a dsRNA HES5 treated sample. Solid arrows show some of the Atoh1 staining, dashed arrows show some of the myosin 7a staining

FIG. 6 shows data that treatment with dsRNA to HES5 down-regulates HES5 mRNA and up-regulates Atoh1 mRNA.

FIGS. 7A-7B shows serum stability results for various dsRNA nucleic acid molecules disclosed herein. FIG. 7A shows stability of two different HES1_{—}14 dsRNAs in mouse and rat serum (3, 8, and 24 hours) and in rat CSF (CS) and HCT116 (HCT1) cell extract (3, 8 and 24 hours) compared to 1 ng untreated duplex (“Box”). FIG. 7B shows serum stability of HES1_{—}36_S2036 duplex in CSF, rat plasma, cell extract and human plasma (3, 8, 24 hours) compared to 1 ng untreated duplex (“B”). FIGS. 8A-8B shows stability of ID3 dsRNA duplexes in HCT116 and CSF (3, 8, 24 hours) compared to 1 ng untreated duplex (“Box”).

FIG. 8A shows serum stability of ID3 dsRNA duplexes in mouse and rat serum (3, 8, 24 hours) compared to 1 ng untreated duplex (“Box”). FIG. 8B shows stability of ID3 dsRNA duplexes in HCT116 and CSF (3, 8, 24 hours) compared to 1 ng untreated duplex (“Box”).

FIG. 9A shows stability of CDKN1B dsRNA in rat CSF and rat plasma. FIG. 9B shows knockdown activity of CDKN1B dsRNA at various concentrations.

FIGS. 10A-10B show plasma, CSF (cerebrospinal fluid) and cell extract stability of 4 different HEY1 dsRNA nucleic acid molecules. FIG. 10A shows stability of 4 dsRNA in mouse (Ms) and rat (Rt) plasma and rat CSF for 3, 8, and 24 hours. FIG. 10B shows stability of the 4 dsRNA in human cell extract (HCT116).

### DETAILED DESCRIPTION OF THE INVENTION

Provided herein are molecules and compositions which down-regulate expression of certain genes associated with hearing loss and their use in treating a subject suffering from hearing loss. In preferred embodiments the methods comprise partial or full hearing regeneration Inhibition of expression of HES1, HES5, HEY1, HEY2, ID1, ID2, ID3, CDKN1B, and NOTCH1, was shown to be beneficial in regeneration of hearing. The present application relates in particular to dsRNA molecules including small interfering RNA (siRNA) compounds which inhibit expression of HES1, HES5, HEY1, HEY2, ID1, ID2, ID3, CDKN1B, and NOTCH1, and to the use of these dsRNA molecules in the treatment of hearing loss. Sense strands and complementary antisense strands useful in generating dsRNA molecules are set forth in SEQ ID NOS:23-26912. Certain currently preferred sense strand and antisense strand pairs are set forth in tables I-IX, supra.

Compounds, compositions and methods for inhibiting HES1, HES5, HEY1, HEY2, ID1, ID2, ID3, CDKN1B or NOTCH1 are discussed herein at length, and any of said compounds and/or compositions may be beneficially employed in the treatment of a patient suffering from a hearing disorder/hearing loss.

The present invention provides methods and compositions for inhibiting expression of a target HES1, HES5, HEY1, HEY2, ID1, ID2, ID3, CDKN1B, or NOTCH1 gene in vivo. In general, the method includes administering oligoribonucleotides, such as dsRNA molecules including small interfering RNAs (i.e., dsRNAs) that are targeted to a particular mRNA selected from any one of SEQ ID NOS:1-11, and hybridize to, or interact with, the mRNAs under biological conditions (within the cell), or a nucleic acid material that can produce siRNA in a cell, in an amount sufficient to down-regulate expression of a target gene by an RNA interference mechanism.

The present invention relates in general to compounds which down-regulate expression of genes expressed in otic cells, for example in the hair cells of the cochlea, particularly to novel small interfering RNAs (siRNAs), and to the use of these novel siRNAs in the treatment of a subject suffering from hearing loss associated with expression of those genes in the ear.

Methods for the delivery of chemically modified dsRNA molecules to ear are discussed herein at length, and any of said molecules and/or compositions may be beneficially employed in the treatment of a subject suffering from hearing loss. Hearing regeneration may be full or partial and is readily determined by one with skill in the art.

The dsRNAs disclosed herein possess structures and modifications which may, for example increase activity, increase stability, and or minimize toxicity; the chemically modified dsRNAs molecules disclosed herein are useful in preventing or attenuating target gene expression, in particular the target genes discussed herein.

Details of a non-limited example of target genes per indication are presented in Table 1A, hereinbelow.

TABLE 1A
Target genes for treatment of hearing loss

| | Gene | |
|---|---|---|
| | abbrevia- | |
| No. | tion | Full name and gi and accession numbers |
| 1 | HES1 | hairy and enhancer of split 1, (Drosophila) |
| | | Alternative Names: bHLHb39; FLJ20408; HES-1; HHL; |
| | | HRY gi\|8400709\|ref\|NM_005524.2\| (SEQ ID NO: 1) |
| 2 | HES5 | hairy and enhancer of split 5 (Drosophila) |
| | | Alternative Names: bHLHb |
| | | 38gi\|145301612\|ref\|NM_001010926.2\| (SEQ ID NO: 2) |
| 3 | ID1 | inhibitor of DNA binding 1, dominant negative helix- |
| | | loop-helix protein. Alternative Names: bHLHb24; ID |
| | | gi\|31317298\|ref\|NM_002165.2\| transcript variant 1 |
| | | (SEQ ID NO: 3) |
| | | gi\|31317296\|ref\|NM_181353.1\| transcript variant 2 |
| | | (SEQ ID NO: 4) |
| 4 | ID2 | inhibitor of DNA binding 2, dominant negative helix- |
| | | loop-helix protein Alternative Names: bHLHb26; GIG8; |
| | | ID2A; ID2H; MGC26389 |
| | | gi\|33946335\|ref\|NM_002166.4\| (SEQ ID NO: 5) |
| 5 | ID3 | inhibitor of DNA binding 3, dominant negative helix- |
| | | loop-helix protein. Alternative Names: bHLHb25; HEIR- |
| | | 1 |
| | | gi\|156119620\|ref\|NM_002167.3 \| (SEQ ID NO: 6) |
| 6 | CDKN1B | cyclin-dependent kinase inhibitor 1B (p27, Kip1) |
| | | Alternative Names: CDKN4; KIP1; MEN1B; MEN4; |
| | | P27KIP1 gi\|17978497\|ref\|NM_004064.2\| (SEQ ID NO: 7) |
| 7 | HEY1 | HEY1—hairy/enhancer-of-split related with YRPW |
| | | motif 1 |
| | | gi\|105990527\|ref\|NM_012258.3\| transcript variant 1 |
| | | (SEQ ID NO: 8) |
| | | gi\|105990525\|ref\|NM_001040708.1\| transcript variant 2 |
| | | (SEQ ID NO: 9) |
| 8 | HEY2 | HEY2—hairy/enhancer-of-split related with YRPW |
| | | motif 2 |
| | | gi\|105990529\|ref\|NM_012259.2\| (SEQ ID NO: 10) |
| 9 | NOTCH1 | NOTCH1 Notch homolog 1, translocation-associated |
| | | (Drosophila) gi\|148833507\|ref\|NM_017617.3\| Homo |
| | | sapiens mRNA (SEQ ID NO: 11) |

Table 1A provides the gi (GeneInfo identifier) and accession numbers for an example of polynucleotide sequences of human mRNA to which the oligonucleotide inhibitors of the present invention are directed. (“v” refers to transcript variant). Inhibition of any one or more of the genes in Table 1A is useful in treating hearing loss and for hearing regeneration.

A brief description of the target genes is as follows:

1. HES1 hairy and enhancer of split 1, (Drosophila). Official Symbol HES1 and Name: hairy and enhancer of split 1, (Drosophila)

Aliases: F1120408, HES-1, HHL, HRY, hairy and enhancer of split 1; hairy homolog; transcription factor HES-1

HES1 protein belongs to the basic helix-loop-helix family of transcription factors. It is a transcriptional repressor of genes that require a bHLH protein for their transcription. Human HES1 mRNA polynucleotide sequence is set forth in SEQ ID NO:1. Human HES1 polypeptide is set forth in SEQ ID NO:12.

2. HES5 hairy and enhancer of split 5 (Drosophila). Official Symbol HES5 and Name: hairy and enhancer of split 5 (Drosophila)

Human HES5 gene is a downstream marker of Notch signaling in articular chondrocytes. Human HES5 mRNA polynucleotide sequence is set forth in SEQ ID NO:2. Human HES5 polypeptide sequence is set forth in SEQ ID NO:13.

3. inhibitor of DNA binding 1, dominant negative helix-loop-helix protein. Official Symbol ID1 and Name: inhibitor of DNA binding 1, dominant negative helix-loop-helix protein [Homo sapiens]. Aliases: ID, DNA-binding protein inhibitor ID-1; dJ857M17.1.2 (inhibitor of DNA binding 1, dominant negative helix-loop-helix protein); inhibitor of DNA binding 1; inhibitor of differentiation 1

ID1 is a helix-loop-helix (HLH) protein that can form heterodimers with members of the basic HLH family of transcription factors. Two transcript variants encoding different isoforms have been found for this gene. Human ID1 mRNA polynucleotide sequences are set forth in SEQ ID NO:3 and 4. Human ID1 polypeptide sequences are set forth in SEQ ID NO:14 and 15, respectively.

4. inhibitor of DNA binding 2, dominant negative helix-loop-helix protein. Official Symbol ID2 and Name: inhibitor of DNA binding 2, dominant negative helix-loop-helix protein [Homo sapiens]. Aliases: GIG8, ID2A, ID2H, MGC26389, DNA-binding protein inhibitor ID2; cell growth-inhibiting gene 8; helix-loop-helix protein ID2; inhibitor of DNA binding 2; inhibitor of differentiation 2

Human ID2 mRNA polynucleotide sequence is set forth in SEQ ID NO:5. Human ID2 polypeptide sequence is set forth in SEQ ID NO:16.

5. inhibitor of DNA binding 3, dominant negative helix-loop-helix protein. Official Symbol ID3 and Name: inhibitor of DNA binding 3, dominant negative helix-loop-helix protein [Homo sapiens]. Aliases: HEIR-1

Human ID3 mRNA polynucleotide sequence is set forth in SEQ ID NO:6. Human ID3 polypeptide sequence is set forth in SEQ ID NO:17.

6. cyclin-dependent kinase inhibitor 1B (p2′7, Kip1). Official Symbol CDKN1B and Name: cyclin-dependent kinase inhibitor 1B (p27, Kip1). Aliases: CDKN4, KIP1, MEN1B, MEN4, P27KIP1

A cyclin-dependent kinase inhibitor, which shares a limited similarity with CDK inhibitor CDKN1A/p21. Human CDKN1B mRNA polynucleotide sequence is set forth in SEQ ID NO:7. Human CDKN1B polypeptide sequence is set forth in SEQ ID NO:18.

7. HEY1—hairy/enhancer-of-split related with YRPW motif 1. Human HEY1 mRNA polynucleotide sequences are set forth in SEQ ID NO:8 and 9. Human HEY1 polypeptide sequences are set forth in SEQ ID NO:19 and 20.

8. HEY2—hairy/enhancer-of-split related with YRPW motif 2. Human HEY2 mRNA polynucleotide sequence is set forth in SEQ ID NO:10. Human HEY1 polypeptide sequence is set forth in SEQ ID NO:21.

9. NOTCH1—Homo sapiens Notch homolog 1, translocation-associated (Drosophila). Human NOTCH1 mRNA polynucleotide sequence is set forth in SEQ ID NO:11. Human NOTCH1 polypeptide sequence is set forth in SEQ ID NO:22.

In various embodiments, provided are double stranded RNA, (dsRNA) including chemically modified small interfering RNAs (siRNAs), and to the use of the dsRNAs in the treatment of various diseases and medical conditions. Particular diseases and conditions to be treated are related to hearing loss. Particular target genes are presented in Table 1A.

Preferred sequences useful in generating dsRNA are provided in SEQ ID NOS: 23-26912. The sequences were prioritized based on their score according to a proprietary algorithm as the best sequences for targeting the human gene expression. SEQ ID NOS:23-693 and 26691-26706 (HES1); SEQ ID NOS:1496-2029 and 26725-26732 (HES5); SEQ ID NOS:2704-3025 and 26809-26816 (ID1); SEQ ID NOS:3634-5053 and 26825-26832 (ID2); SEQ ID NOS:6206-6671 and 26851-26866 (ID3); SEQ ID NOS:7444-9007 and 26887-26900 (CDKN1B); SEQ ID NOS:10534-11549 and 26761-26778 (HEY1); SEQ ID NOS:13004-14801 and 26785-26788 (HEY2); SEQ ID NOS:16622-18643 and 26922-26912 (NOTCH1) set forth 19-mer oligomers. SEQ ID NOS:694-1495 (HES1); SEQ ID NOS:2030-2703 (HES5); SEQ ID NOS:3026-3633 (ID1); SEQ ID NOS:5054-6205 (ID2); SEQ ID NOS:6672-7443 (ID3); SEQ ID NOS:9008-10533 (CDKN1B); SEQ ID NOS:11550-13003 (HEY1); SEQ ID NOS:14802-16389 (HEY2); SEQ ID NOS:18644-26666 (NOTCH1) set forth 18-mer oligomers Useful in generating dsRNA molecules according to Structure A2.

Disclosed herein are compounds which down-regulate expression of HES1, HES5, HEY1, HEY2, ID1, ID2, ID3, CDKN1B, or NOTCH1, particularly to novel chemically modified double stranded RNA oligonucleotides (dsRNAs), and to the use of these novel dsRNAs in the treatment of various diseases and medical conditions, particularly diseases and disorders of the ear. According to one aspect the present invention provides inhibitory oligonucleotide compounds comprising unmodified and modified nucleotides and or unconventional moieties. The compound includes at least one modified nucleotide selected from the group consisting of a sugar modification, a base modification and an internucleotide linkage modification and may further include DNA, and modified nucleotides or unconventional moieties including LNA (locked nucleic acid), ENA (ethylene-bridged nucleic acid), PNA (peptide nucleic acid), arabinoside, PACE, mirror nucleotide, a nucleotide joined to an adjacent nucleotide by a 2′-5′ internucleotide bond or a nucleotide with a 6 carbon sugar.

Methods, nucleic acid molecules and compositions, which down-regulate HES1, HES5, HEY1, HEY2, ID1, ID2, ID3 or CDKN1B are discussed herein at length, and any of said molecules and/or compositions may be beneficially employed in the treatment of a subject suffering from any of said conditions.

The nucleic acid compounds provided herein possess structures and modifications, which may increase activity, increase stability, and or minimize toxicity; the novel modifications useful in generating dsRNA compounds disclosed herein can be beneficially applied to double stranded RNA useful in preventing or attenuating HES1, HES5, HEY1, HEY2, ID1, ID2, ID3, CDKN1B or NOTCH1 expression.

In some embodiments provided herein is a double stranded RNA compound having the structure (A1):

| | | |
|---|---|---|
| (A1) | 5′ (N)x-Z 3′ | (antisense strand) |
| | 3′ Z′-(N′)y-z″ 5′ | (sense strand) |

wherein each N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein each of Z and Z′ is independently present or absent, but if present independently comprises 1-5 consecutive nucleotides, 1-5 consecutive non-nucleotide moieties or a combination thereof covalently attached at the 3′ terminus of the strand in which it is present;
wherein z″ may be present or absent, but if present is a capping moiety covalently attached at the 5′ terminus of (N′)y;
each of x and y is independently an integer from 18 to 40;
   wherein the sequence of (N′)y is complementary to the sequence of (N)x; and wherein (N)x comprises an antisense sequence and (N′)y comprises a sense sequence set forth in SEQ ID NOS:23-693 and 26691-26706 (HES1); SEQ ID NOS:1496-2029 and 26725-26732 (HES5); SEQ ID NOS:2704-3025 and 26809-26816 (ID1); SEQ ID NOS:3634-5053 and 26825-26832 (ID2); SEQ ID NOS:6206-6671 and 26851-26866 (ID3); SEQ ID NOS:7444-9007 and 26887-26900 (CDKN1B); SEQ ID NOS:10534-11549 and 26761-26778 (HEY1); SEQ ID NOS:13004-14801 and 26785-26788 (HEY2); SEQ ID NOS:16622-18643 and 26922-26912 (NOTCH1).

Preferred (N)x and (N′)y are set forth in any one of SEQ ID NOS:26691-26706 (HES1); SEQ ID NOS:26725-26732 (HES5); SEQ ID NOS: 26809-26816 (ID1); SEQ ID NOS:26825-26832 (ID2); SEQ ID NOS:26851-26866 (ID3); SEQ ID NOS:26887-26900 (CDKN1B); SEQ ID NOS:26761-26778 (HEY1); SEQ ID NOS:26785-26788 (HEY2); SEQ ID NOS:26922-26912 (NOTCH1).
In some embodiments the covalent bond joining each consecutive N and/or N′ is a phosphodiester bond.

In some embodiments x=y and each of x and y is 19, 20, 21, 22 or 23. In preferred embodiments x=y=19.

In some embodiments of nucleic acid molecules (e.g., dsRNA molecules) as disclosed herein, the double stranded nucleic acid molecule is a siRNA, siNA or a miRNA.

In some embodiments the double stranded nucleic acid molecules comprise a DNA moiety or a mismatch to the target at position 1 of the antisense strand (5′ terminus). Such a duplex structure is described herein. According to one embodiment provided are double stranded dsRNA molecules having a structure (A2) set forth below:

| | | |
|---|---|---|
| (A2) | 5′ N1-(N)x-Z 3′ | (antisense strand) |
| | 3′ Z′-N2-(N′)y-z″ 5′ | (sense strand) |

wherein each N1, N2, N and N′ is independently an unmodified or modified nucleotide, or an unconventional moiety;
wherein each of (N)x and (N′)y is an oligonucleotide in which each consecutive N or N′ is joined to the adjacent N or N′ by a covalent bond;
wherein each of x and y is independently an integer between 17 and 39;
wherein N2 is covalently bound to (N′)y;
wherein N1 is covalently bound to (N)x and is mismatched to the target mRNA (SEQ ID NO:1-11) or is a complementary DNA moiety to the target mRNA;
wherein N1 is a moiety selected from the group consisting of natural or modified: uridine, deoxyribouridine, ribothymidine, deoxyribothymidine, adenosine or deoxyadenosine, an abasic ribose moiety and an abasic deoxyribose moiety;
wherein z″ may be present or absent, but if present is a capping moiety covalently attached at the 5′ terminus of N2-(N′)y;
wherein each of Z and Z′ is independently present or absent, but if present is independently 1-5 consecutive nucleotides, 1-5 consecutive non-nucleotide moieties or a combination thereof covalently attached at the 3′ terminus of the strand in which it is present; and

wherein the sequence of (N′)y is complementary to the sequence of (N)x; and wherein the sequence of (N)x comprises an antisense sequence and (N′)y comprises a sense sequence set forth in SEQ ID NOS:694-1495 (HES1); SEQ ID NOS:2030-2703 (HES5); SEQ ID NOS:3026-3633 (ID1); SEQ ID NOS:5054-6205 (ID2); SEQ ID NOS:6672-7443 (ID3); SEQ ID NOS:9008-10533 (CDKN1B); SEQ ID NOS:11550-13003 (HEY1); SEQ ID NOS:14802-16389 (HEY2); SEQ ID NOS:18644-26666 (NOTCH1).

Preferred N1-(N)x and N2-(N′)y are set forth in any one of SEQ ID NOS:26667-26690 (HES1); SEQ ID NOS:26707-26724 (HES5); SEQ ID NOS:26789-26808 (ID1); SEQ ID NOS:26817-26824 (ID2); SEQ ID NOS: 26833-26850 (ID3); SEQ ID NOS:26867-26886 (CDKN1B); SEQ ID NOS:26733-26760 (HEY1); SEQ ID NOS:26779-26784 (HEY2); SEQ ID NOS:2601-26910 (NOTCH1).

### DEFINITIONS

For convenience certain terms employed in the specification, examples and claims are described herein.

It is to be noted that, as used herein, the singular forms “a”, “an” and “the” include plural forms unless the content clearly dictates otherwise.

Where aspects or embodiments of the invention are described in terms of Markush groups or other grouping of alternatives, those skilled in the art will recognize that the invention is also thereby described in terms of any individual member or subgroup of members of the group.

An “inhibitor” is a compound, which is capable of reducing (partially or fully) the expression of a gene or the activity of the product of such gene to an extent sufficient to achieve a desired biological or physiological effect. The term “inhibitor” as used herein refers to one or more of an oligonucleotide inhibitor, including siRNA, shRNA, synthetic shRNA; miRNA, antisense RNA and DNA and ribozymes.

A “dsRNA molecule” or “dsRNA inhibitor” is a compound which is capable of down-regulating or reducing the expression of a gene or the activity of the product of such gene to an extent sufficient to achieve a desired biological or physiological effect and includes one or more of a siRNA, shRNA, synthetic shRNA; miRNA. Inhibition may also be referred to as down-regulation or, for RNAi, silencing.

The term “inhibit” as used herein refers to reducing the expression of a gene or the activity of the product of such gene to an extent sufficient to achieve a desired biological or physiological effect. Inhibition is either complete or partial.

As used herein, the term “inhibition” of a target gene means inhibition of the gene expression (transcription or translation) or polypeptide activity of a target gene wherein the target gene is selected from a gene transcribed into an mRNA set forth in any one of SEQ ID NOS:1-11 or an SNP (single nucleotide polymorphism) or other variants thereof. The gi number for the mRNA of each target gene is set forth in Table 1A. The polynucleotide sequence of the target mRNA sequence, or the target gene having a mRNA sequence refer to the mRNA sequences set forth in SEQ ID NO:1-11, or any homologous sequences thereof preferably having at least 70% identity, more preferably 80% identity, even more preferably 90% or 95% identity to any one of mRNA set forth in SEQ ID NO:1-11. Therefore, polynucleotide sequences derived from any one of SEQ ID NO:1-11 which have undergone mutations, alterations or modifications as described herein are encompassed in the present invention. The terms “mRNA polynucleotide sequence”, “mRNA sequence” and “mRNA” are used interchangeably.

As used herein, the terms “polynucleotide” and “nucleic acid” may be used interchangeably and refer to nucleotide sequences comprising deoxyribonucleic acid (DNA), and ribonucleic acid (RNA). The terms are to be understood to include, as equivalents, analogs of either RNA or DNA made from nucleotide analogs. Throughout this application, mRNA sequences are set forth as representing the corresponding genes.

“Oligonucleotide” or “oligomer” refers to a deoxyribonucleotide or ribonucleotide sequence from about 2 to about 50 nucleotides. Each DNA or RNA nucleotide may be independently natural or synthetic, and or modified or unmodified. Modifications include changes to the sugar moiety, the base moiety and or the linkages between nucleotides in the oligonucleotide. The compounds of the present invention encompass molecules comprising deoxyribonucleotides, ribonucleotides, modified deoxyribonucleotides, modified ribonucleotides and combinations thereof.

“Substantially complementary” refers to complementarity of greater than about 84%, to another sequence. For example in a duplex region consisting of 19 base pairs one mismatch results in 94.7% complementarity, two mismatches results in about 89.5% complementarity and 3 mismatches results in about 84.2% complementarity, rendering the duplex region substantially complementary. Accordingly substantially identical refers to identity of greater than about 84%, to another sequence.

“Nucleotide” is meant to encompass deoxyribonucleotides and ribonucleotides, which may be natural or synthetic, and or modified or unmodified. Modifications include changes to the sugar moiety, the base moiety and or the linkages between ribonucleotides in the oligoribonucleotide. As used herein, the term “ribonucleotide” encompasses natural and synthetic, unmodified and modified ribonucleotides. Modifications include changes to the sugar moiety, to the base moiety and/or to the linkages between ribonucleotides in the oligonucleotide.

The nucleotides can be selected from naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine. In some embodiments one or more nucleotides in an oligomer is substituted with inosine.

According to some embodiments the present invention provides inhibitory oligonucleotide compounds comprising unmodified and modified nucleotides and or unconventional moieties. The compound comprises at least one modified nucleotide selected from the group consisting of a sugar modification, a base modification and an internucleotide linkage modification and may contain DNA, and modified nucleotides such as LNA (locked nucleic acid), ENA (ethylene-bridged nucleic acid), PNA (peptide nucleic acid), arabinoside, phosphonocarboxylate or phosphinocarboxylate nucleotide (PACE nucleotide), mirror nucleotide, or nucleotides with a 6 carbon sugar.

All analogs of, or modifications to, a nucleotide/oligonucleotide are employed with the present invention, provided that said analog or modification does not substantially adversely affect the function of the nucleotide/oligonucleotide. Acceptable modifications include modifications of the sugar moiety, modifications of the base moiety, modifications in the internucleotide linkages and combinations thereof.

A sugar modification includes a modification on the 2′ moiety of the sugar residue and encompasses amino, fluoro, alkoxy e.g. methoxy, alkyl, amino, fluoro, chloro, bromo, CN, CF, imidazole, carboxylate, thioate, C₁ to C₁₀ lower alkyl, substituted lower alkyl, alkaryl or aralkyl, OCF₃, OCN, O—, S—, or N-alkyl; O-, S, or N-alkenyl; SOCH₃; SO₂CH₃; ONO₂; NO₂, N₃; heterozycloalkyl; heterozycloalkaryl; aminoalkylamino; polyalkylamino or substituted silyl, as, among others, described in European patents EP 0 586 520 B1 or EP 0 618 925 B1.

In one embodiment the dsRNA molecule comprises at least one ribonucleotide comprising a 2′ modification on the sugar moiety (“2′ sugar modification”). In certain embodiments the compound comprises 2′O-alkyl or 2′-fluoro or 2′O-allyl or any other 2′ modification, optionally on alternate positions. Other stabilizing modifications are also possible (e.g. terminal modifications). In some embodiments a preferred 2′O-alkyl is 2′O-methyl (methoxy) sugar modification.

In some embodiments the backbone of the oligonucleotides is modified and comprises phosphate-D-ribose entities but may also contain thiophosphate-D-ribose entities, triester, thioate, 2′-5′ bridged backbone (also may be referred to as 5′-2′), PACE and the like.

As used herein, the terms “non-pairing nucleotide analog” means a nucleotide analog which comprises a non-base pairing moiety including but not limited to: 6 des amino adenosine (Nebularine), 4-Me-indole, 3-nitropyrrole, 5-nitroindole, Ds, Pa, N3-Me ribo U, N3-Me riboT, N3-Me dC, N3-Me-dT, N1-Me-dG, N1-Me-dA, N3-ethyl-dC, N3-Me dC. In some embodiments the non-base pairing nucleotide analog is a ribonucleotide. In other embodiments it is a deoxyribonucleotide. In addition, analogues of polynucleotides may be prepared wherein the structure of one or more nucleotide is fundamentally altered and better suited as therapeutic or experimental reagents. An example of a nucleotide analogue is a peptide nucleic acid (PNA) wherein the deoxyribose (or ribose) phosphate backbone in DNA (or RNA) is replaced with a polyamide backbone which is similar to that found in peptides. PNA analogues have been shown to be resistant to enzymatic degradation and to have extended stability in vivo and in vitro. Other modifications that can be made to oligonucleotides include polymer backbones, cyclic backbones, acyclic backbones, thiophosphate-D-ribose backbones, triester backbones, thioate backbones, 2′-5′ bridged backbone, artificial nucleic acids, morpholino nucleic acids, glycol nucleic acid (GNA), threose nucleic acid (TNA), arabinoside, and mirror nucleoside (for example, beta-L-deoxyribonucleoside instead of beta-D-deoxyribonucleoside). Examples of dsRNA molecules comprising LNA nucleotides are disclosed in Elmen et al., (NAR 2005, 33(1):439-447).

The compounds of the present invention can be synthesized using one or more inverted nucleotides, for example inverted thymidine or inverted adenine (see, for example, Takei, et al., 2002, JBC 277(26):23800-06).

A “mirror” nucleotide is a nucleotide with reversed chirality to the naturally occurring or commonly employed nucleotide, i.e., a mirror image (L-nucleotide) of the naturally occurring (D-nucleotide), also referred to as L-RNA in the case of a mirror ribonucleotide, and “spiegelmer”. The nucleotide can be a ribonucleotide or a deoxyribonucleotide and my further comprise at least one sugar, base and or backbone modification. See U.S. Pat. No. 6,586,238. Also, U.S. Pat. No. 6,602,858 discloses nucleic acid catalysts comprising at least one L-nucleotide substitution.

Other modifications include terminal modifications on the 5′ and/or 3′ part of the oligonucleotides and are also known as capping moieties. Such terminal modifications are selected from a nucleotide, a modified nucleotide, a lipid, a peptide, a sugar and inverted abasic moiety.

An “alkyl moiety or derivative thereof” refers to straight chain or branched carbon moieties and moieties per se or further comprising a functional group including alcohols, phosphodiester, phosphorothioate, phosphonoacetate and also includes amines, carboxylic acids, esters, amides aldehydes. “Hydrocarbon moiety” and “alkyl moiety” are used interchangeably.

“Terminal functional group” includes halogen, alcohol, amine, carboxylic, ester, amide, aldehyde, ketone, ether groups.

Provided are methods and compositions for inhibiting expression of the target gene in vivo. In general, the method includes administering oligoribonucleotides, in particular small interfering RNAs (i.e. siRNAs) that target an mRNA transcribed from the target gene in an amount sufficient to down-regulate expression of a target gene by an RNA interference mechanism. In particular, the subject method can be used to inhibit expression of the target gene for treatment of a disease. Provided herein are dsRNA molecules directed to a target gene disclosed herein and useful as therapeutic agents to treat various otic and vestibular system pathologies.

Provided are methods and compositions for inhibiting expression of a hearing loss-associated gene in vivo. In general, the method includes administering oligoribonucleotides, in particular double stranded RNAs (such as, for example, siRNAs), that target an mRNA, or pharmaceutical compositions comprising them, in an amount sufficient to down-regulate expression of a target gene by an RNA interference mechanism. In particular, the subject method can be used to inhibit expression of a hearing loss-associated gene for treatment of a disease or a disorder or a condition disclosed herein.

Disclosed herein are chemically modified dsRNA compounds, which down-regulate the expression of a target gene transcribed into mRNA having a polynucleotide sequence set forth in any one of SEQ ID NOS:1-11 and pharmaceutical compositions comprising one or more such compounds.

Provided herein are methods and compositions for inhibiting expression of HES1, in vivo. Provided herein are methods and compositions for inhibiting expression of HES5, in vivo. Provided herein are methods and compositions for inhibiting expression of HEY1, in vivo. Provided herein are methods and compositions for inhibiting expression of HEY2, in vivo. Provided herein are methods and compositions for inhibiting expression of ID1, in vivo. Provided herein are methods and compositions for inhibiting expression of ID2, in vivo. Provided herein are methods and compositions for inhibiting expression of ID3, in vivo. Provided herein are methods and compositions for inhibiting expression of CDKN1B, in vivo. Provided herein are methods and compositions for inhibiting expression of NOTCH1, in vivo. In general, the method includes administering oligoribonucleotides, in particular double stranded RNAs (i.e. dsRNAs) or a nucleic acid material that can produce dsRNA in a cell, that target an mRNA transcribed from HES1, HES5, HEY1, HEY2, ID1, ID2, ID3, CDKN1B or NOTCH1 gene in an amount sufficient to down-regulate expression of HES1, HES5, HEY1, HEY2, ID1, ID2, ID3, CDKN1B, or NOTCH1 by e.g., an RNA interference mechanism. In particular, the subject method can be used to down-regulate expression of HES1, HES5, HEY1, HEY2, ID1, ID2, ID3, CDKN1B, or NOTCH1 for treatment of a disease, disorder or injury. In accordance with the present invention, the nucleic acid molecules or inhibitors of HES1, HES5, HEY1, HEY2, ID1, ID2, ID3, CDKN1B, or NOTCH1 are used as drugs to treat various pathologies. In accordance with the present invention, the nucleic acid molecules or inhibitors of HES1, HES5, HEY1, HEY2, ID1, ID2, ID3, CDKN1B, or NOTCH1 are used as drugs to treat disease or disorder in the ear.

A dsRNA of the invention is a duplex oligoribonucleotide in which the sense strand is substantially complementary to an 18-40 consecutive nucleotide segment of the mRNA polynucleotide sequence of a target gene, and the antisense strand is substantially complementary to the sense strand. In general, some deviation from the target mRNA sequence is tolerated without compromising the siRNA activity (see e.g. Czauderna et al., Nuc. Acids Res. 2003, 31(11):2705-2716). A siRNA of the invention inhibits gene expression on a post-transcriptional level with or without destroying the mRNA. Without being bound by theory, siRNA may target the mRNA for specific cleavage and degradation and/or may inhibit translation from the targeted message.

In some embodiments the dsRNA is blunt ended, on one or both ends. More specifically, the dsRNA may be blunt ended on the end defined by the 5′-terminus of the first strand and the 3′-terminus of the second strand, or the end defined by the 3′-terminus of the first strand and the 5′-terminus of the second strand.

In other embodiments at least one of the two strands may have an overhang of at least one nucleotide at the 5′-terminus; the overhang may consist of at least one deoxyribonucleotide. At least one of the strands may also optionally have an overhang of at least one nucleotide at the 3′-terminus. The overhang may consist of from about 1 to about 5 nucleotides.

The length of RNA duplex is from about 18 to about 40 ribonucleotides, preferably 19 to 23 ribonucleotides. Further, the length of each strand (oligomer) may independently have a length selected from the group consisting of about 18 to about 40 bases, preferably 18 to 23 bases and more preferably 19, 20 or 21 ribonucleotides.

Additionally, in certain preferred embodiments the complementarity between said first strand and the target nucleic acid is perfect. In some embodiments, the strands are substantially complementary, i.e. having one, two or up to three mismatches between said first strand and the target nucleic acid.

Further, the 5′-terminus of the first strand of the siRNA may be linked to the 3′-terminus of the second strand, or the 3′-terminus of the first strand may be linked to the 5′-terminus of the second strand, said linkage being via a nucleic acid linker typically having a length between 3-100 nucleotides, preferably about 3 to about 10 nucleotides.

The siRNAs compounds of the present invention possess structures and modifications which impart one or more of increased activity, increased stability, reduced toxicity, reduced off target effect, and/or reduced immune response. The siRNA structures of the present invention are beneficially applied to double stranded RNA useful in preventing or attenuation target gene expression, in particular the target genes discussed herein.

According to one aspect the present invention provides a chemically modified double stranded oligonucleotide comprising at least one modified nucleotide selected from the group consisting of a sugar modification, a base modification and an internucleotide linkage modification. Accordingly, the chemically modified double stranded oligonucleotide compounds of the invention may contain modified nucleotides such as DNA, LNA (locked nucleic acid), ENA (ethylene-bridged nucleic acid), PNA (peptide nucleic acid), arabinoside, PACE, mirror nucleoside, or nucleotides with a 6 carbon sugar. Examples of PACE nucleotides and analogs are disclosed in U.S. Pat. Nos. 6,693,187 and 7,067,641 both incorporated herein by reference. The oligonucleotide may further comprise 2′O-methyl or 2′-fluoro or 2′O-allyl or any other 2′ modification, optionally on alternate positions. Other stabilizing modifications, which do not significantly reduce the activity are also possible (e.g. terminal modifications). The backbone of the active part of the oligonucleotides may comprise phosphate-D-ribose entities but may also contain thiophosphate-D-ribose entities, triester, thioate, 2′-5′ bridged backbone (also may be referred to as 5′-2′), PACE or any other type of modification. Terminal modifications on the 5′ and/or 3′ part of the oligonucleotides are also possible. Such terminal modifications may be lipids, peptides, sugars, inverted abasic moieties or other molecules.

The present invention also relates to compounds which down-regulate expression of the genes disclosed herein, particularly to novel ds RNAs, and to the use of these novel dsRNAs in the treatment of various diseases and medical conditions. Particular diseases and conditions to be treated are related to hearing loss or disorders disclosed herein. Lists of siRNA to be used in the present invention are provided in Tables 2A-10D. 21- or 23-mer siRNA sequences can also be generated by 5′ and/or 3′ extension of the 19-mer sequences disclosed herein. Such extension is preferably complementary to the corresponding mRNA sequence. The dsRNAs of the present invention possess structures and modifications which may increase activity, increase stability, reduce off-target effect, reduce immune response and/or reduce toxicity. The dsRNA structures of the present invention are beneficially applied to double stranded RNA useful in preventing or attenuating expression of one or more of the target genes disclosed herein.

Methods, molecules and compositions of the present invention which inhibit the genes disclosed herein are discussed herein at length, and any of said molecules and/or compositions are beneficially employed in the treatment of a subject suffering from one or more of said conditions.

Where aspects or embodiments of the invention are described in terms of Markush groups or other grouping of alternatives, those skilled in the art will recognize that the invention is also thereby described in terms of any individual member or subgroup of members of the group.

### The Human Ear

The ear is comprised of three major structural components: the outer, middle, and inner ears, which function together to convert sound waves into nerve impulses that travel to the brain, where they are perceived as sound. The inner ear also helps to maintain balance.

The anatomy of the middle and the inner ear is well known to those of ordinary skill in the art (see, e.g., Atlas of Sensory Organs: Functional and Clinical Analysis, Andrs Csillag, Humana Press (2005), pages 1-82, incorporated herein by reference). In brief, the middle ear consists of the eardrum and a small air-filled chamber containing a sequence of three tiny bones known as the ossicles, which link the eardrum to the inner ear.

The inner ear (labyrinth) is a complex structure consisting of the cochlea, which is the organ of hearing and the vestibular system, the organ of balance. The vestibular system consists of the saccule and the utricle, which determine position sense, and the semicircular canals, which help maintain balance.

The cochlea houses the organ of Corti, which consists, in part, of about 20,000 specialized sensory cells, called “inner ear hair cells” or “hair cells”. These cells have small hairline projections (cilia) that extend into the cochlear fluid. Sound vibrations transmitted from the ossicles in the middle ear to the oval window in the inner ear cause the fluid and cilia to vibrate. Hair cells in different parts of the cochlea vibrate in response to different sound frequencies and convert the vibrations into nerve impulses which are sent to the brain for processing and interpretation. The inner ear hair cells are surrounded by inner ear support cells. Supporting cells underlie, at least partially surround, and physically support sensory hair cells within the inner ear. Representative examples of support cells include inner rod (pillar cells), outer rod (pillar cells), inner phalangeal cells, outer phalangeal cells (of Deiters), cells of Held, cells of Hensen, cells of Claudius, cells of Boettcher, interdental cells and auditory teeth (of Huschke).

The spiral ganglion is the group of nerve cells that send a representation of sound from the cochlea to the brain. The cell bodies of the spiral ganglion neurons are found in the spiral structure of the cochlea and are part of the central nervous system. Their dendrites make synaptic contact with the base of hair cells, and their axons are bundled together to form the auditory portion of the eighth cranial nerve (vestibulocochlear nerve).

### Hearing Loss

Auditory hair cells are sensory receptors located in the organ of Corti of the cochlea involved in detecting sound. The cochlear hair cells come in two anatomically and functionally distinct types: the outer and inner hair cells. Auditory hair cells convert sound information into electrical signals that are sent via nerve fibers to the brain and processed.

Vestibular hair cells, located in the vestibular organs of the inner ear (utricle, saccule, ampullae), detect changes in head position and convey this information to the brain to help maintain balance posture and eye position.

In the absence of auditory hair cells, sound waves are not converted into neural signals and hearing deficits ensue, for example, decreased hearing sensitivity, i.e. sensorineural hearing loss. In the absence of vestibular hair cells, balance deficits ensue.

Despite the protective effect of the acoustic reflex, loud noise can damage and destroy hair cells. Irreversible hair cell death is elicited by metabolic or biochemical changes in the hair cells that involve reactive oxygen species (ROS). Exposure to certain drugs and continued exposure to loud noise, inter alia, cause progressive damage, eventually resulting in ringing in the ears (tinnitus) and or hearing loss.

Acquired hearing loss can be caused by several factors including exposure to harmful noise levels, exposure to ototoxic drugs such as cisplatin and aminoglycoside antibiotics and aging.

U.S. Ser. No. 11/655,610 to the assignee of the present invention relates to methods of treating hearing impairment by inhibiting a pro-apoptotic gene in general and p53 in particular. International Patent Publication No. WO 2005/119251 relates to methods of treating deafness. International Patent Publication No. WO/2005/055921 relates to foam compositions for treatment of ear disorders. U.S. Pat. No. 7,087,581 relates to methods of treating diseases and disorders of the inner ear. PCT Publication No. WO 2009/147684, assigned to the assignee of the present application, and incorporated herein by reference in its entirety discloses certain compounds and compositions for treating otic disorders and diseases.

dsRNA and RNA Interference

RNA interference (RNAi) is a phenomenon involving double-stranded (ds) RNA-dependent gene-specific posttranscriptional silencing. Initial attempts to study this phenomenon and to manipulate mammalian cells experimentally were frustrated by an active, non-specific antiviral defense mechanism which was activated in response to long dsRNA molecules (Gil et al., Apoptosis, 2000. 5:107-114). Later, it was discovered that synthetic duplexes of 21 nucleotide RNAs could mediate gene specific RNAi in mammalian cells, without stimulating the generic antiviral defense mechanisms Elbashir et al. Nature 2001, 411:494-498 and Caplen et al. PNAS 2001, 98:9742-9747). As a result, small interfering RNAs (siRNAs), which are short double-stranded RNAs, have been widely used to inhibit gene expression and understand gene function.

RNA interference (RNAi) is mediated by small interfering RNAs (siRNAs) (Fire et al, Nature 1998, 391:806) or microRNAs (miRNAs) (Ambros V. Nature 2004, 431:350-355); and Bartel D P. Cell. 2004 116(2):281-97). The corresponding process is commonly referred to as specific post-transcriptional gene silencing when observed in plants and as quelling when observed in fungi.

A siRNA compound is a double-stranded RNA which down-regulates or silences (i.e. fully or partially inhibits) the expression of an endogenous or exogenous gene/mRNA. RNA interference is based on the ability of certain dsRNA species to enter a specific protein complex, where they are then targeted to complementary cellular RNAs and specifically degrades them. Thus, the RNA interference response features an endonuclease complex containing an siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir, et al., Genes Dev., 2001, 15:188). In more detail, longer dsRNAs are digested into short (17-29 bp) dsRNA fragments (also referred to as short inhibitory RNAs or “siRNAs”) by type III RNAses (DICER, DROSHA, etc., (see Bernstein et al., Nature, 2001, 409:363-6 and Lee et al., Nature, 2003, 425:415-9). The RISC protein complex recognizes these fragments and complementary mRNA. The whole process is culminated by endonuclease cleavage of target mRNA (McManus and Sharp, Nature Rev Genet, 2002, 3:737-47; Paddison and Hannon, Curr Opin Mol. Ther. 2003, 5(3): 217-24). (For additional information on these terms and proposed mechanisms, see for example, Bernstein, et al., RNA. 2001, 7(11):1509-21; Nishikura, Cell. 2001, 107(4):415-8 and PCT Publication No. WO 01/36646).

The selection and synthesis of dsRNA compounds corresponding to known genes has been widely reported; see for example Ui-Tei et al., J Biomed Biotechnol. 2006; 65052; Chalk et al., BBRC. 2004, 319(1):264-74; Sioud and Leirdal, Met. Mol. Biol.; 2004, 252:457-69; Levenkova et al., Bioinform. 2004, 20(3):430-2; Ui-Tei et al., NAR 2004, 32(3):936-48. For examples of the use of, and production of, modified siRNA see Braasch et al., Biochem., 2003, 42(26):7967-75; Chiu et al., RNA, 2003, 9(9):1034-48; PCT publications WO 2004/015107 (atugen); WO 02/44321 (Tuschl et al), and U.S. Pat. Nos. 5,898,031 and 6,107,094.

Several groups have described the development of DNA-based vectors capable of generating siRNA within cells. The method generally involves transcription of short hairpin RNAs that are efficiently processed to form siRNAs within cells (Paddison et al. PNAS USA 2002, 99:1443-1448; Paddison et al. Genes & Dev 2002, 16:948-958; Sui et al. PNAS USA 2002, 8:5515-5520; and Brummelkamp et al. Science 2002, 296:550-553). These reports describe methods of generating siRNAs capable of specifically targeting numerous endogenously and exogenously expressed genes.

Studies have revealed that siRNA can be effective in vivo in both mammals and humans. Specifically, Bitko et al., showed that specific siRNAs directed against the respiratory syncytial virus (RSV) nucleocapsid N gene are effective in treating mice when administered intranasally (Nat. Med. 2005, 11(1):50-55). For reviews of therapeutic applications of siRNAs see for example Barik (Mol. Med. 2005, 83: 764-773) and Chakraborty (Current Drug Targets 2007 8(3):469-82). In addition, clinical studies with short siRNAs that target the VEGFR1 receptor in order to treat age-related macular degeneration (AMD) have been conducted in human patients (Kaiser, Am J Ophthalmol. 2006 142(4):660-8). Further information on the use of siRNA as therapeutic agents may be found in Durcan, 2008. Mol. Pharma. 5(4):559-566; Kim and Rossi, 2008. BioTechniques 44:613-616; Grimm and Kay, 2007, JCI, 117(12):3633-41.

### Chemical Synthesis

The compounds of the present invention can be synthesized by any of the methods that are well-known in the art for synthesis of ribonucleic (or deoxyribonucleic) oligonucleotides. Such synthesis is, among others, described in Beaucage and Iyer, Tetrahedron 1992; 48:2223-2311; Beaucage and Iyer, Tetrahedron 1993; 49: 6123-6194 and Caruthers, et. al., Methods Enzymol. 1987; 154: 287-313; the synthesis of thioates is, among others, described in Eckstein, Annu Rev. Biochem. 1985; 54: 367-402, the synthesis of RNA molecules is described in Sproat, in Humana Press 2005 edited by Herdewijn P.; Kap. 2: 17-31 and respective downstream processes are, among others, described in Pingoud et. al., in IRL Press 1989 edited by Oliver R.W.A.; Kap. 7: 183-208.

Other synthetic procedures are known in the art e.g. the procedures as described in Usman et al., 1987, J. Am. Chem. Soc., 109, 7845; Scaringe et al., 1990, NAR., 18, 5433; Wincott et al., 1995, NAR. 23, 2677-2684; and Wincott et al., 1997, Methods Mol. Bio., 74, 59, and these procedures may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

The oligonucleotides of the present invention can be synthesized separately and joined together post-synthetically, for example, by ligation (Moore et al., 1992, Science 256, 9923; Draper et al., International Patent Publication No. WO 93/23569; Shabarova et al., 1991, NAR 19, 4247; Bellon et al., 1997, Nucleosides & Nucleotides, 16, 951; Bellon et al., 1997, Bioconjugate Chem. 8, 204), or by hybridization following synthesis and/or deprotection.

It is noted that a commercially available machine (available, inter alia, from Applied Biosystems) can be used; the oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present invention, two or more such sequences can be synthesized and linked together for use in the present invention.

The compounds of the invention can also be synthesized via tandem synthesis methodology, as described for example in US Patent Publication No. US 2004/0019001 (McSwiggen), wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker.

The present invention further provides for a pharmaceutical composition comprising two or more siRNA molecules for the treatment of any of the diseases and conditions mentioned herein, whereby said two molecules may be physically mixed together in the pharmaceutical composition in amounts which generate equal or otherwise beneficial activity, or may be covalently or non-covalently bound, or joined together by a nucleic acid linker of a length ranging from 2-100, preferably 2-50 or 2-30 nucleotides. In one embodiment, the siRNA molecules are comprised of a double-stranded nucleic acid structure as described herein, wherein the two dsRNA molecules are selected from the oligonucleotides described herein. Thus, the siRNA molecules may be covalently or non-covalently bound or joined by a linker to form a tandem siRNA compound. Such tandem dsRNA molecules comprising two siRNA sequences are typically of 38-150 nucleotides in length, more preferably 38 or 40-60 nucleotides in length, and longer accordingly if more than two siRNA sequences are included in the tandem molecule. A longer tandem compound comprised of two or more longer sequences which encode siRNA produced via internal cellular processing, e.g., long dsRNAs, is also envisaged, as is a tandem molecule encoding two or more shRNAs. Such tandem molecules are also considered to be a part of the disclosure. A compound comprising two (tandem) or more (RNAistar) dsRNA sequences disclosed herein is envisaged. Examples of such “tandem” or “star” molecules are provided in PCT patent publication no. WO 2007/091269, assigned to the assignee of the present application and incorporated herein by reference in its entirety.

The dsRNA molecules that target HEST, HES5, HEY1, HEY2, ID1, ID2, ID3, CDKN1B, or NOTCH1 may be the main active component in a pharmaceutical composition, or may be one active component of a pharmaceutical composition containing two or more dsRNAs (or molecules which encode or endogenously produce two or more dsRNAs, be it a mixture of molecules or one or more tandem molecules which encode two or more dsRNAs), said pharmaceutical composition further being comprised of one or more additional dsRNA molecule which targets one or more additional gene. Simultaneous inhibition of said additional gene(s) will likely have an additive or synergistic effect for treatment of the diseases disclosed herein.

Additionally, the dsRNA disclosed herein or any nucleic acid molecule comprising or encoding such dsRNA can be linked or bound (covalently or non-covalently) to antibodies (including aptamer molecules) against cell surface internalizable molecules expressed on the target cells, in order to achieve enhanced targeting for treatment of the diseases disclosed herein. For example, anti-Fas antibody (preferably a neutralizing antibody) may be combined (covalently or non-covalently) with any dsRNA. In another example, an aptamer which can act like a ligand/antibody may be combined (covalently or non-covalently) with any dsRNA.

The nucleic acid molecules disclosed herein can be delivered either directly or with viral or non-viral vectors. When delivered directly, the sequences are generally rendered nuclease resistant. Alternatively the sequences can be incorporated into expression cassettes or constructs such that the sequence is expressed in the cell as discussed herein below. Generally the construct contains the proper regulatory sequence or promoter to allow the sequence to be expressed in the targeted cell. Vectors optionally used for delivery of the compounds of the present invention are commercially available, and may be modified for the purpose of delivery of the compounds of the present invention by methods known to one of skill in the art.

### Chemical Modifications

All analogs of, or modifications to, a nucleotide/oligonucleotide may be employed with the present invention, provided that said analogue or modification does not substantially affect the function of the nucleotide/oligonucleotide. The nucleotides can be selected from naturally occurring or synthetic modified bases. Naturally occurring bases include adenine, guanine, cytosine, thymine and uracil. Modified bases of nucleotides are described herein.

In addition, analogues of polynucleotides can be prepared wherein the structure of one or more nucleotide is fundamentally altered and better suited as therapeutic or experimental reagents. An example of a nucleotide analogue is a peptide nucleic acid (PNA) wherein the deoxyribose (or ribose) phosphate backbone in DNA (or RNA is replaced with a polyamide backbone which is similar to that found in peptides. PNA analogues have been shown to be resistant to enzymatic degradation and to have extended stability in vivo and in vitro. Other modifications that can be made to oligonucleotides include polymer backbones, cyclic backbones, acyclic backbones, thiophosphate-D-ribose backbones, triester backbones, thioate backbones, 2′-5′ bridged backbone, artificial nucleic acids, morpholino nucleic acids, locked nucleic acid (LNA), glycol nucleic acid (GNA), threose nucleic acid (TNA), arabinoside, and mirror nucleoside (for example, beta-L-deoxynucleoside instead of beta-D-deoxynucleoside). Examples of dsRNA molecules comprising LNA nucleotides are disclosed in Elmen et al., (NAR 2005, 33(1):439-447).

The nucleic acid compounds of the present invention can be synthesized using one or more inverted nucleotides, for example inverted thymidine or inverted adenine (see, for example, Takei, et al., 2002, JBC 277(26):23800-06).

The term “unconventional moiety” as used herein refers to abasic ribose moiety, an abasic deoxyribose moiety, a deoxyribonucleotide, a modified deoxyribonucleotide, a mirror nucleotide, a non-base pairing nucleotide analog and a nucleotide joined to an adjacent nucleotide by a 2′-5′ internucleotide phosphate bond; C3, C4, C5 and C6 moieties; bridged nucleic acids including LNA and ethylene bridged nucleic acids.

The term “capping moiety” as used herein includes abasic ribose moiety, abasic deoxyribose moiety, modifications of abasic ribose and abasic deoxyribose moieties including 2′ O alkyl modifications; inverted abasic ribose and abasic deoxyribose moieties and modifications thereof; C6-imino-Pi; a mirror nucleotide including L-DNA and L-RNA; 5′OMe nucleotide; and nucleotide analogs including 4′,5′-methylene nucleotide; 1-(β-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non bridging methylphosphonate and 5′-mercapto moieties.

Abasic deoxyribose moiety includes for example abasic deoxyribose-3′-phosphate; 1,2-dideoxy-D-ribofuranose-3-phosphate; 1,4-anhydro-2-deoxy-D-ribito1-3-phosphate. Inverted abasic deoxyribose moiety includes inverted deoxyriboabasic; 3′,5′ inverted deoxyriboabasic 5′-phosphate.

A “mirror” nucleotide is a nucleotide with reversed chirality to the naturally occurring or commonly employed nucleotide, i.e., a mirror image (L-nucleotide) of the naturally occurring (D-nucleotide). The nucleotide can be a ribonucleotide or a deoxyribonucleotide and may further comprise at least one sugar, base and/or backbone modification. U.S. Pat. No. 6,602,858 discloses nucleic acid catalysts comprising at least one L-nucleotide substitution. Mirror nucleotide includes for example L-DNA (L-deoxyriboadenosine-3′-phosphate (mirror dA); L-deoxyribocytidine-3′-phosphate (mirror dC); L-deoxyriboguanosine-3′-phosphate (mirror dG); L-deoxyribothymidine-3′-phosphate (mirror image dT)) and L-RNA (L-riboadenosine-3′-phosphate (mirror rA); L-ribocytidine-3′-phosphate (mirror rC); L-riboguanosine-3′-phosphate (mirror rG); L-ribouracil-3′-phosphate (mirror dU).

In various embodiments of Structure A1 or Structure A2, Z and Z′ are absent. In other embodiments Z or Z′ is present. In some embodiments each of Z and/or Z′ independently includes a C2, C3, C4, C5 or C6 alkyl moiety, optionally a C3 [propane, —(CH2)3-] moiety or a derivative thereof including propanol (C3-OH/C3OH), propanediol, and phosphodiester derivative of propanediol (“C3Pi”). In preferred embodiments each of Z and/or Z′ includes two hydrocarbon moieties and in some examples is C3Pi-C3OH or C3Pi-C3Pi. Each C3 is covalently conjugated to an adjacent C3 via a covalent bond, preferably a phospho-based bond. In some embodiments the phospho-based bond is a phosphorothioate, a phosphonoacetate or a phosphodiester bond.

In specific embodiments of Structure A1 x=y=19 and Z comprises at least one C3 alkyl overhang. In specific embodiments of Structure A2 x=y=18 and Z comprises at least one C3 alkyl overhang. In some embodiments the C3-C3 overhang is covalently attached to the 3′ terminus of (N)x or (N′)y via a covalent linkage, preferably a phosphodiester linkage. In some embodiments the linkage between a first C3 and a second C3 is a phosphodiester linkage. In some embodiments the 3′ non-nucleotide overhang is C3Pi-C3Pi. In some embodiments the 3′ non-nucleotide overhang is C3Pi-C3Ps. In some embodiments the 3′ non-nucleotide overhang is C3Pi-C3OH(OH is hydroxy). In some embodiments the 3′ non-nucleotide overhang is C3Pi-C3OH.

In various embodiments the alkyl moiety comprises an alkyl derivative including a C3 alkyl, C4 alkyl, C5 alkyl or C6 alkyl moiety comprising a terminal hydroxyl, a terminal amino, or terminal phosphate group. In some embodiments the alkyl moiety is a C3 alkyl or C3 alkyl derivative moiety. In some embodiments the C3 alkyl moiety comprises propanol, propylphosphate, propylphosphorothioate or a combination thereof. The C3 alkyl moiety is covalently linked to the 3′ terminus of (N′)y and/or the 3′ terminus of (N)x via a phosphodiester bond. In some embodiments the alkyl moiety comprises propanol, propyl phosphate or propyl phosphorothioate. In some embodiments each of Z and Z′ is independently selected from propanol, propyl phosphate propyl phosphorothioate, combinations thereof or multiples thereof in particular 2 or 3 covalently linked propanol, propyl phosphate, propyl phosphorothioate or combinations thereof. In some embodiments each of Z and Z′ is independently selected from propyl phosphate, propyl phosphorothioate, propyl phospho-propanol; propyl phospho-propyl phosphorothioate; propylphospho-propyl phosphate; (propyl phosphate)3, (propyl phosphate)2-propanol, (propyl phosphate)2-propyl phosphorothioate. Any propane or propanol conjugated moiety can be included in Z or Z′.

The structures of exemplary 3′ terminal non-nucleotide moieties are as follows:

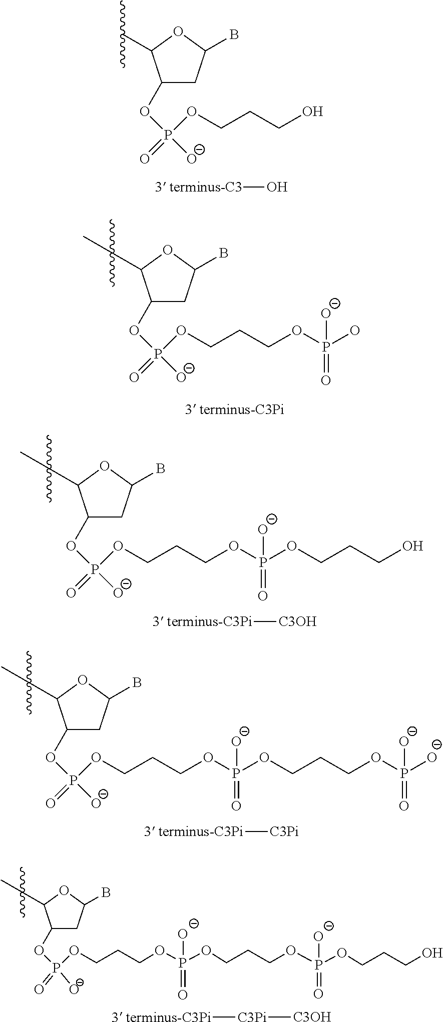

### Indications

The molecules and compositions disclosed herein are useful in the treatment of diseases and disorders of the ear, as well as other diseases and conditions herein described.

### Ear Disorders

The present invention is directed to compositions and methods useful in treating a patient suffering from or at risk of various ear disorders. Ear disorders include hearing loss induced for example by ototoxins, excessive noise or ageing. Middle and inner ear disorders produce many of the same symptoms, and a disorder of the middle ear may affect the inner ear and vice versa.

In addition to hearing loss, ear disorders include myringitis, an eardrum infection caused by a variety of viruses and bacteria; temporal bone fracture for example due to a blow to the head; auditory nerve tumors (acoustic neuroma, acoustic neurinoma, vestibular schwannoma, eighth nerve tumor).

In various embodiments, the methods and compositions disclosed herein are useful in treating various conditions of hearing loss. Without being bound by theory, the hearing loss may be due to apoptotic inner ear hair cell damage or loss (Zhang et al., Neuroscience 2003. 120:191-205; Wang et al., J. Neuroscience 23((24):8596-8607), wherein the damage or loss is caused by infection, mechanical injury, loud sound (noise), aging (presbycusis), or chemical-induced ototoxicity.

By “ototoxin” in the context disclosed herein is meant a substance that through its chemical action injures, impairs or inhibits the activity of the sound receptors component of the nervous system related to hearing, which in turn impairs hearing (and/or balance). In the context of the present invention, ototoxicity includes a deleterious effect on the inner ear hair cells. Ototoxins include therapeutic drugs including antineoplastic agents, salicylates, loop-diuretics, quinines, and aminoglycoside antibiotics, contaminants in foods or medicinals, and environmental or industrial pollutants. Typically, treatment is performed to prevent or reduce ototoxicity, especially resulting from or expected to result from administration of therapeutic drugs. Preferably a composition comprising a therapeutically effective amount of a chemically modified siRNA compound of the invention is given immediately after the exposure to prevent or reduce the ototoxic effect. More preferably, treatment is provided prophylactically, either by administration of the pharmaceutical composition of the invention prior to or concomitantly with the ototoxic pharmaceutical or the exposure to the ototoxin. Incorporated herein by reference are chapters 196, 197, 198 and 199 of The Merck Manual of Diagnosis and Therapy, 14th Edition, (1982), Merck Sharp & Dome Research Laboratories, N.J. and corresponding chapters in the most recent 16th edition, including Chapters 207 and 210) relating to description and diagnosis of hearing and balance impairments.

Accordingly, in one aspect provided are methods and pharmaceutical compositions for treating a mammal, preferably human, to prevent, reduce, or treat a hearing impairment, disorder or imbalance, preferably an ototoxin-induced hearing condition, by administering to a mammal in need of such treatment a chemically modified siRNA compound of the invention. One embodiment is directed to a method for treating a hearing disorder or impairment wherein the ototoxicity results from administration of a therapeutically effective amount of an ototoxic pharmaceutical drug. Typical ototoxic drugs are chemotherapeutic agents, e.g. antineoplastic agents, and antibiotics. Other possible candidates include loop-diuretics, quinines or a quinine-like compound, PDE-5 inhibitors and salicylate or salicylate-like compounds.

Ototoxicity is a dose-limiting side effect of antibiotic administration. From 4 to 15% of patients receiving 1 gram per day for greater than 1 week develop measurable hearing loss, which slowly becomes worse and can lead to complete permanent deafness if treatment continues. Ototoxic aminoglycoside antibiotics include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. Particular antibiotics include neomycin B, kanamycin A, kanamycin B, gentamicin C1, gentamicin C1a, and gentamicin C2, and the like that are known to have serious toxicity, particularly ototoxicity and nephrotoxicity, which reduce the usefulness of such antimicrobial agents (see Goodman and Gilman's The Pharmacological Basis of Therapeutics, 6th ed., A. Goodman Gilman et al., eds; Macmillan Publishing C0., Inc., New York, pp. 1169-71 (1980)).

Ototoxicity is also a serious dose-limiting side-effect for anti-cancer agents. Ototoxic neoplastic agents include but are not limited to vincristine, vinblastine, cisplatin and cisplatin-like compounds and taxol and taxol-like compounds. Cisplatin-like compounds include carboplatin (Paraplatin®), tetraplatin, oxaliplatin, aroplatin and transplatin inter alia and are platinum based chemotherapeutics.

Diuretics with known ototoxic side-effect, particularly “loop” diuretics include, without being limited to, furosemide, ethacrylic acid, and mercurials.

Ototoxic quinines include but are not limited to synthetic substitutes of quinine that are typically used in the treatment of malaria. In some embodiments the hearing disorder is side-effect of inhibitors of type 5 phosphodiesterase (PDE-5), including sildenafil (Viagra®), vardenafil (Levitra®) and tadalafil (Clalis).

Salicylates, such as aspirin, are the most commonly used therapeutic drugs for their anti-inflammatory, analgesic, anti-pyretic and anti-thrombotic effects. Unfortunately, they too have ototoxic side effects. They often lead to tinnitus (“ringing in the ears”) and temporary hearing loss. Moreover, if the drug is used at high doses for a prolonged time, the hearing impairment can become persistent and irreversible.

In some embodiments a method is provided for treatment of infection of a mammal by administration of an aminoglycoside antibiotic, the improvement comprising administering a therapeutically effective amount of one or more chemically modified siRNAs compounds which down-regulate expression a target gene, to the subject in need of such treatment to reduce or prevent ototoxin-induced hearing impairment associated with the antibiotic.

The molecules and pharmaceutical compositions described herein are also effective in the treatment of acoustic trauma or mechanical trauma, preferably acoustic or mechanical trauma that leads to inner ear hair cell loss. With more severe exposure, injury can proceed from a loss of adjacent supporting cells to complete disruption of the organ of Corti. Death of the sensory cell can lead to progressive Wallerian degeneration and loss of primary auditory nerve fibers. The methods provided are useful in treating acoustic trauma caused by a single exposure to an extremely loud sound, or following long-term exposure to everyday loud sounds above 85 decibels, for treating mechanical inner ear trauma, for example, resulting from the insertion of an electronic device into the inner ear or for preventing or minimizing the damage to inner ear hair cells associated with the operation.

Another type of hearing loss is presbycusis, which is hearing loss that gradually occurs in most individuals as they age. About 30-35 percent of adults between the ages of 65 and 75 years and 40-50 percent of people 75 and older experience hearing loss. The methods of the invention are useful in preventing, reducing or treating the incidence and/or severity of inner ear disorders and hearing impairments associated with presbycusis.

### Acoustic Trauma

Acoustic trauma is a type of hearing loss that is caused by prolonged exposure to loud noises. Without wishing to be bound to theory, exposure to loud noise causes the hair cells on the cochlea to become less sensitive. With more severe exposure, injury can proceed from a loss of adjacent supporting cells to complete disruption of the organ of Corti. Death of the sensory cell can lead to progressive Wallerian degeneration and loss of primary auditory nerve fibers. Disclosed herein are molecules, pharmaceutical compositions and methods useful in attenuating hearing loss due to acoustic trauma. Provided are dsRNA molecules that target any one of HES1, HES5, HEY1, HEY2, ID1, ID2, ID3, CDKN1B, or NOTCH1 for treating or preventing acoustic trauma in a subject exposed to acoustic trauma.

Provided is a method of treating a subject suffering from or at risk of an ear disorder which comprises topically administering to the canal of the subject's ear a pharmaceutical composition comprising an oligonucleotide inhibitor and a pharmaceutically acceptable excipient or mixtures thereof, thereby reducing expression of a gene associated with the disorder in the ear of the subject in an amount effective to treat the subject. Further provided is a method of treating a subject suffering from or at risk of an ear disorder which comprises transtympanically administering to the canal of the subject's ear a pharmaceutical composition comprising an oligonucleotide inhibitor and a pharmaceutically acceptable excipient or mixtures thereof, thereby reducing expression of a gene associated with the disorder in the ear of the subject in an amount effective to treat the subject. In one embodiment, the dsRNA is delivered via a posterior semicircular canalostomy. In one embodiment, the dsRNA is delivered as ear drops.

In some embodiments, the pharmaceutical composition is applied to the ear canal when the subject's head is tilted to one side and the treated ear is facing upward. In some embodiments, the pharmaceutical composition is applied to the ear using a receptacle for eardrops, for example using a dropper of for example, 10-100 microliter per drop, or a wick.

In some embodiments an ear disorder relates to chemical-induced hearing loss; for example hearing loss induced by inter alia cisplatin and its analogs; aminoglycoside antibiotics, quinine and its analogs; salicylate and its analogs; phosphodiesterase type 5 (PDE5) inhibitors or loop-diuretics. In some embodiments the ear disorder refers to noise-induced hearing loss. In other embodiments the ear disorder is age related hearing loss.

Without being bound by theory, inhibition of HES1, HES5, HEY1, HEY2, ID1, ID2, ID3, CDKN1B, or NOTCH1 can cause hair cell regeneration, optionally via an increase in Atoh1 expression. The compounds of the present invention are useful in treating, ameliorating or preventing any disease, disorder or injury in which promoting proliferation of supporting cells or outer or inner hair cells in the cochlea is required; optionally related with ototoxin-induced hearing loss.

### Diseases and Disorders of the Vestibular System

In various embodiments the nucleic acid compounds and pharmaceutical compositions of the invention are useful for treating disorders and diseases affecting the vestibular system in which expression of HES1, HES5, HEY1, HEY2, ID1, ID2, ID3, CDKN1B, or NOTCH1 is detrimental, for example Meniere's Disease. The vestibular sensory system in most mammals, including humans, contributes to balance, and to a sense of spatial orientation and stability. Together with the cochlea it constitutes the labyrinth of the inner ear. The vestibular system comprises two components: the semicircular canal system, which indicate rotational movements; and the otoliths, which indicate linear accelerations.

### Meniere's Disease

Ménière's disease, also known as idiopathic endolymphatic hydrops (ELH), is a disorder of the inner ear resulting in vertigo and tinnitus, and eventual neuronal damage leading to hearing loss. The exact cause of Ménière's disease is unknown but the underlying mechanism is believed to be distortion of the membranous labyrinth due to accumulation of endolymph. Endolymph is produced primarily by the stria vascularis in the cochlea and also by the planum semilunatum and the dark cells in the vestibular labyrinth (Sajjadi H, Paparella M M. Meniere's disease. Lancet. 372(9636):406-14). If the flow of endolymph from the endolymphatic fluid space through the vestibular aqueduct to the endolymphatic sac is obstructed, endolymphatic hydrops will occur. Meniere's disease may affect one or both of a subject's ears. The primary morbidity associated with Meniere's disease is the debilitating nature of vertigo and the progressive hearing loss. Current therapies have not been successful at preventing progression of neuronal degeneration and associated hearing loss. A therapeutic treatment, which would protect the neurons of the inner ear including the vestibulocochlear nerve from damage and/or induce regeneration of the vestibulocochlear nerve and thereby attenuate or prevent hearing loss in Meniere's patients would be highly desirable.

The nucleic acids, compositions, methods and kits provided herein are useful in treating subjects at risk of or suffering from Meniere's disease.

In conclusion, there are no effective modes of therapy for the prevention and/or treatment of the conditions disclosed herein. Treatments that are available suffer from, inter alia, the drawbacks of severe side effects due to the lack of selective targeting and there remains a need therefore to develop novel compounds and methods of treatment for these purposes.

In various embodiments the compounds and pharmaceutical compositions of the invention are useful in treating or preventing various diseases, disorders and injury that affect the ear, such as, without being limited to, the diseases, disorders and injury that are disclosed herein below. Without being bound by theory, it is believed that the molecules of the present invention prevent death or various types of cells within the ear.

### Pharmaceutical Compositions

Provided are compositions and methods for down-regulation of HES1, HES5, HEY1, HEY2, ID1, ID2, ID3, CDKN1B, or NOTCH1 expression by using small nucleic acid molecules, such as short interfering nucleic acid (siNA), interfering RNA (RNAi), short interfering RNA (siRNA), double-stranded RNA (dsRNA), micro-RNA (miRNA), and short hairpin RNA (shRNA) molecules capable of mediating down-regulation of HES1, HES5, HEY1, HEY2, ID1, ID2, ID3, CDKN1B, or NOTCH1 gene expression or that mediate RNA interference against HES1, HES5, HEY1, HEY2, ID1, ID2, ID3, CDKN1B, or NOTCH1 gene expression.

While it may be possible for the molecules disclosed herein to be administered as the raw chemical, it is preferable to present them as a pharmaceutical composition. Accordingly provided is a pharmaceutical composition comprising one or more of the dsRNA molecules disclosed herein; and a pharmaceutically acceptable carrier. This composition may comprise a mixture of two or more different nucleic acid compounds.

Compositions, methods and kits provided herein may include one or more nucleic acid molecules (e.g., dsRNA) and methods that independently or in combination modulate (e.g., down-regulate) the expression of HES1, HES5, HEY1, HEY2, ID1, ID2, ID3, CDKN1B, or NOTCH1 protein and/or genes encoding HES1, HES5, HEY1, HEY2, ID1, ID2, ID3, CDKN1B, or NOTCH1 protein, proteins and/or genes associated with the maintenance and/or development of diseases, conditions or disorders associated with HES1, HES5, HEY1, HEY2, ID1, ID2, ID3, CDKN1B, or NOTCH1, particularly disorders associated with the ear. The description of the various aspects and embodiments is provided with reference to exemplary genes HES1, HES5, HEY1, HEY2, ID1, ID2, ID3, CDKN1B, or NOTCH1. However, the various aspects and embodiments are also directed to other related genes, such as homolog genes and transcript variants, and polymorphisms (e.g., single nucleotide polymorphism, (SNPs)) associated with certain HES1, HES5, HEY1, HEY2, ID1, ID2, ID3, CDKN1B, or NOTCH1 genes. As such, the various aspects and embodiments are also directed to other genes that are involved in HES1, HES5, HEY1, HEY2, ID1, ID2, ID3, CDKN1B, or NOTCH1 mediated pathways of signal transduction or gene expression that are involved, for example, in the maintenance or development of diseases, traits, or conditions described herein. These additional genes can be analyzed for target sites using the methods described for the HES1, HES5, HEY1, HEY2, ID1, ID2, ID3, CDKN1B, or NOTCH1 gene herein. Thus, the down-regulation of other genes and the effects of such modulation of the other genes can be performed, determined, and measured as described herein.

Further provided is a pharmaceutical composition comprising at least one compound of the invention covalently or non-covalently bound to one or more compounds of the invention in an amount effective to down regulate HES1, HES5, HEY1, HEY2, ID1, ID2, ID3, CDKN1B, or NOTCH1 expression; and a pharmaceutically acceptable carrier. The compound may be processed intracellularly by endogenous cellular complexes to produce one or more oligoribonucleotides of the invention.

Further provided is a pharmaceutical composition comprising a pharmaceutically acceptable carrier and one or more of the compounds of the invention in an amount effective to inhibit expression in a cell of human HES1, HES5, HEY1, HEY2, ID1, ID2, ID3, CDKN1B, or NOTCH1, the compound comprising a sequence which is substantially complementary to the sequence of (N)x.

Substantially complementary refers to complementarity of greater than about 84%, to another sequence. For example in a duplex region consisting of 19 base pairs one mismatch results in 94.7% complementarity, two mismatches results in about 89.5% complementarity and 3 mismatches results in about 84.2% complementarity, rendering the duplex region substantially complementary. Accordingly substantially identical refers to identity of greater than about 84%, to another sequence.

Additionally, the invention provides a method of inhibiting the expression of HES1, HES5, HEY1, HEY2, ID1, ID2, ID3, CDKN1B, or NOTCH1 by at least 20%, by at least 30% by at least 40%, preferably by 50%, 60% or 70%, more preferably by 75%, 80% or 90% as compared to a control comprising contacting an mRNA transcript of the present invention with one or more of the compounds of the invention.

In one embodiment the oligoribonucleotide compounds, compositions and methods disclosed herein inhibit/down-regulate the HES1, HES5, HEY1, HEY2, ID1, ID2, ID3, CDKN1B, or NOTCH1 gene, whereby the inhibition/down-regulation is selected from the group comprising inhibition/down-regulation of gene function, inhibition/down-regulation of polypeptide and inhibition/down-regulation of mRNA expression.

In one embodiment, compositions and methods provided herein include a double-stranded short interfering nucleic acid (siNA) compound that down-regulates expression of a HES1, HES5, HEY1, HEY2, ID1, ID2, ID3, CDKN1B gene (e.g., the mRNA coding sequence for human HES1, HES5, HEY1, HEY2, ID1, ID2, ID3, CDKN1B, or NOTCH1 exemplified by SEQ ID NO:1-11), where the nucleic acid molecule includes about 15 to about 49 base pairs.

In one embodiment, a nucleic acid disclosed herein may be used to inhibit the expression of the HES1, HES5, HEY1, HEY2, ID1, ID2, ID3, CDKN1B or NOTCH1 gene or a HES1, HES5, HEY1, HEY2, ID1, ID2, ID3, CDKN1B, or NOTCH1 gene family where the genes or gene family sequences share sequence homology. Such homologous sequences can be identified as is known in the art, for example using sequence alignments. Nucleic acid molecules can be designed to target such homologous sequences, for example using perfectly complementary sequences or by incorporating non-canonical base pairs, for example mismatches and/or wobble base pairs, that can provide additional target sequences. In instances where mismatches are identified, non-canonical base pairs (for example, mismatches and/or wobble bases) can be used to generate nucleic acid molecules that target more than one gene sequence. In a non-limiting example, non-canonical base pairs such as UU and CC base pairs are used to generate nucleic acid molecules that are capable of targeting sequences for differing HES1, HES5, HEY1, HEY2, ID1, ID2, ID3, CDKN1B, or NOTCH1 targets that share sequence homology. As such, one advantage of using dsRNAs disclosed herein is that a single nucleic acid can be designed to include nucleic acid sequence that is complementary to the nucleotide sequence that is conserved between the homologous genes. In this approach, a single nucleic acid can be used to inhibit expression of more than one gene instead of using more than one nucleic acid molecule to target the different genes.

Nucleic acid molecules may be used to target conserved sequences corresponding to a gene family or gene families such as HES1, HES5, HEY1, HEY2, ID1, ID2, ID3, CDKN1B, or NOTCH1 family genes. As such, nucleic acid molecules targeting multiple HES1, HES5, HEY1, HEY2, ID1, ID2, ID3, CDKN1B, or NOTCH1 targets can provide increased therapeutic effect. In addition, nucleic acid can be used to characterize pathways of gene function in a variety of applications. For example, nucleic acid molecules can be used to inhibit the activity of target gene(s) in a pathway to determine the function of uncharacterized gene(s) in gene function analysis, mRNA function analysis, or translational analysis. The nucleic acid molecules can be used to determine potential target gene pathways involved in various diseases and conditions toward pharmaceutical development. The nucleic acid molecules can be used to understand pathways of gene expression involved in, for example ear disorders.

In one embodiment the nucleic acid compounds, compositions and methods provided herein, inhibit the HES1, HES5, HEY1, HEY2, ID1, ID2, ID3, CDKN1B, or NOTCH1 polypeptide, whereby the inhibition is selected from the group comprising inhibition of function (which may be examined by an enzymatic assay or a binding assay with a known interactor of the native gene/polypeptide, inter alia), down-regulation of protein or inhibition of protein (which may be examined by Western blotting, ELISA or immuno-precipitation, inter alia) and inhibition of mRNA expression (which may be examined by Northern blotting, quantitative RT-PCR, in-situ hybridisation or microarray hybridisation, inter alia).

In one embodiment, the compositions and methods provided herein include a nucleic acid molecule having RNAi activity against HES1, HES5, HEY1, HEY2, ID1, ID2, ID3, CDKN1B, or NOTCH1 RNA, where the nucleic acid molecule includes a sequence complementary to any RNA having HES1, HES5, HEY1, HEY2, ID1, ID2, ID3, CDKN1B, or NOTCH1 encoding sequence, such as that sequence set forth in SEQ ID NO: 1-11. In another embodiment, a nucleic acid molecule may have RNAi activity against HES1, HES5, HEY1, HEY2, ID1, ID2, ID3, CDKN1B, or NOTCH1 RNA, where the nucleic acid molecule includes a sequence complementary to an RNA having variant HES1, HES5, HEY1, HEY2, ID1, ID2, ID3, CDKN1B, or NOTCH1 encoding sequence, for example other mutant HES1, HES5, HEY1, HEY2, ID1, ID2, ID3, CDKN1B, or NOTCH1 genes not shown in SEQ ID NO: 1-11 but known in the art to be associated with the onset and/or maintenance and/or development of neurodegeneration and/or neuropathy, for example a SNP. Chemical modifications as described herein can be applied to any nucleic acid construct disclosed herein. In another embodiment, a nucleic acid molecule disclosed herein includes a nucleotide sequence that can interact with nucleotide sequence of a HES1, HES5, HEY1, HEY2, ID1, ID2, ID3, CDKN1B, or NOTCH1 gene and thereby mediate down-regulation or silencing of HES1, HES5, HEY1, HEY2, ID1, ID2, ID3, CDKN1B, or NOTCH1 gene expression, for example, wherein the nucleic acid molecule mediates regulation of HES1, HES5, HEY1, HEY2, ID1, ID2, ID3, CDKN1B, or NOTCH1 gene expression by cellular processes that modulate the chromatin structure or methylation patterns of the gene and prevent transcription of the gene.

More particularly, provided are double stranded nucleic acid molecules wherein one strand includes consecutive nucleotides having, from 5′ to 3′, the compounds set forth in SEQ ID NOS:23-26912 or a homologs thereof wherein in up to two of the ribonucleotides in each terminal region is altered.

### Delivery and Formulations

The RNA molecule of the present invention may be delivered to the ear by direct application of pharmaceutical composition to the outer ear; by transtympanic injection or by ear drops. In some embodiments the pharmaceutical composition is applied to the ear canal. Delivery to the ear may also be refereed to as aural or otic delivery comprising siRNA; a penetration enhancer and a pharmaceutically acceptable vehicle.

Nucleic acid molecules of the present invention may be delivered to the target tissue by direct application of the naked molecules prepared with a carrier or a diluent.

The terms “naked nucleic acid” or “naked dsRNA” or “naked siRNA” refers to nucleic acid molecules that are free from any delivery vehicle that acts to assist, promote or facilitate entry into the cell, including viral sequences, viral particles, liposome formulations, lipofectin or precipitating agents and the like. For example, dsRNA in PBS is “naked dsRNA”.

Nucleic acid molecules disclosed herein may be delivered or administered directly with a carrier or diluent that acts to assist, promote or facilitate entry to the cell, including viral vectors, viral particles, liposome formulations, lipofectin or precipitating agents and the like.

A nucleic acid molecule may include a delivery vehicle, including liposomes, for administration to a subject, carriers and diluents and their salts, and/or can be present in pharmaceutically acceptable formulations. In some embodiments the dsRNA molecules of the invention are delivered in liposome formulations and lipofectin formulations and the like and can be prepared by methods well known to those skilled in the art. Such methods are described, for example, in U.S. Pat. Nos. 5,593,972, 5,589,466, and 5,580,859, which are herein incorporated by reference.

Delivery systems aimed specifically at the enhanced and improved delivery of siRNA into mammalian cells have been developed, (see, for example, Shen et al., FEBS Let. 2003, 539:111-114; Xia et al., Nat. Biotech. 2002, 20:1006-1010; Reich et al., Mol. Vision. 2003, 9: 210-216; Sorensen et al., J. Mol. Biol. 2003. 327: 761-766; Lewis et al., Nat. Gen. 2002, 32: 107-108 and Simeoni et al., NAR 2003, 31, 11: 2717-2724). siRNA has recently been successfully used for inhibition of gene expression in primates (see for example, Tolentino et al., Retina 24(4):660).

Delivery of naked or formulated RNA molecules to the ear, optionally the inner ear, is accomplished, inter alia, by transtympanic injection or by administration of the desired compound formulated as an ear drop. Otic compositions comprising dsRNA are disclosed in US Publication No. 20110142917, to the assignee of the present application and incorporated herein by reference in its entirety.

Polypeptides that facilitate introduction of nucleic acid into a desired subject are known in the art, e.g. such as those described in US. Application Publication No. 20070155658 (e.g., a melamine derivative such as 2,4,6-Triguanidino Traizine and 2,4,6-Tramidosarcocyl Melamine, a polyarginine polypeptide, and a polypeptide including alternating glutamine and asparagine residues).

The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention and they include liposomes and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

In a particular embodiment, the administration comprises transtympanic administration. In another embodiment the administration comprises topical or local administration. The compounds are administered as eardrops, ear cream, ear ointment, foam, mousse or any of the above in combination with a delivery device. Implants of the compounds are also useful. Liquid forms are prepared as drops. The liquid compositions include aqueous solutions, with and without organic co-solvents, aqueous or oil suspensions, emulsions with edible oils, as well as similar pharmaceutical vehicles. These compositions may also be injected transtympanically. Eardrops may also be referred to as otic drops or aural drops. In a preferred embodiment, the ear drops remain in the ear canal for about 30 min in order to prevent leakage of the drops out of the canal. It is thus preferable that the subject receiving the drops keep his head on the side with the treated ear facing upward to prevent leakage of the drop out of the canal.

Methods for the delivery of nucleic acid molecules are described in Akhtar et al., Trends Cell Bio., 2: 139 (1992); Delivery Strategies for Antisense Oligonucleotide Therapeutics, ed. Akhtar, (1995), Maurer et al., Mol. Membr. Biol., 16: 129-140 (1999); Hofland and Huang, Handb. Exp. Pharmacol., 137: 165-192 (1999); and Lee et al., ACS Symp. Ser., 752: 184-192 (2000); U.S. Pat. Nos. 6,395,713; 6,235,310; 5,225,182; 5,169,383; 5,167,616; 4,959,217; 4.925,678; 4,487,603; and 4,486,194 and Sullivan et al., PCT WO 94/02595; PCT WO 00/03683 and PCT WO 02/08754; and U.S. Patent Application Publication No. 2003077829. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see e.g., Gonzalez et al., Bioconjugate Chem., 10: 1068-1074 (1999); Wang et al., International PCT publication Nos. WO 03/47518 and WO 03/46185), poly(lactic-co-glycolic)acid (PLGA) and PLCA microspheres (see for example U.S. Pat. No. 6,447,796 and U.S. Application Publication No. 2002130430), biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether intravitreal, subcutaneous, transtympanic, intramuscular, or intradermal, can take place using standard needle and syringe methodologies, or by needle-free technologies such as those described in Conry et al., Clin. Cancer Res., 5: 2330-2337 (1999) and Barry et al., International PCT Publication No. WO 99/31262. The molecules of the instant invention can be used as pharmaceutical agents. Pharmaceutical agents prevent, modulate the occurrence, or treat or alleviate a symptom to some extent (preferably all of the symptoms) of a disease state in a subject. In one specific embodiment of this invention topical and transdermal formulations may be selected.

The dsRNAs or pharmaceutical compositions of the present invention are administered and dosed in accordance with good medical practice, taking into account the clinical condition of the individual subject, the disease to be treated, the site and method of administration, scheduling of administration, patient age, sex, body weight and other factors known to medical practitioners.

In another embodiment the administration comprises topical or local administration such as via eye drops, eardrops or ointment. In a non-limiting example, dsRNA compounds that target HEST, HES5, HEY1, HEY2, ID1, ID2, ID3, CDKN1B, or NOTCH1 are useful in treating a subject suffering from damage to the ear, wherein the dsRNA compounds are delivered to the ear via topical delivery (e.g., ear drops or ointments). Nucleic acid molecules may be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers. Preferred oligonucleotides useful in generating dsRNA molecules are disclosed herein.

Delivery systems may include surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011).

Nucleic acid molecules may be formulated or complexed with polyethylenimine (e.g., linear or branched PEI) and/or polyethylenimine derivatives, including for example polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives, grafted PEIs such as galactose PEI, cholesterol PEI, antibody derivatized PEI, and polyethylene glycol PEI (PEG-PEI) derivatives thereof (see for example Ogris et al., 2001, AAPA PharmSci, 3, 1-11; Furgeson et al., 2003, Bioconjugate Chem., 14, 840-847; Kunath et al., 2002, Pharmaceutical Research, 19, 810-817; Choi et al., 2001, Bull. Korean Chem. Soc., 22, 46-52; Bettinger et al., 1999, Bioconjugate Chem., 10, 558-561; Peterson et al., 2002, Bioconjugate Chem., 13, 845-854; Erbacher et al., 1999, Journal of Gene Medicine Preprint, 1, 1-18; Godbey et al., 1999., PNAS USA, 96, 5177-5181; Godbey et al., 1999, Journal of Controlled Release, 60, 149-160; Diebold et al., 1999, Journal of Biological Chemistry, 274, 19087-19094; Thomas and Klibanov, 2002, PNAS USA, 99, 14640-14645; Sagara, U.S. Pat. No. 6,586,524 and US Patent Application Publication No. 20030077829).

Nucleic acid molecules may be complexed with membrane disruptive agents such as those described in U.S. Patent Application Publication No. 20010007666. The membrane disruptive agent or agents and the nucleic acid molecule may also be complexed with a cationic lipid or helper lipid molecule, such as those lipids described in U.S. Pat. No. 6,235,310.

Nucleic acid molecules disclosed herein may be administered to the central nervous system (CNS) or peripheral nervous system (PNS). Experiments have demonstrated the efficient in vivo uptake of nucleic acids by neurons. See e.g., Sommer et al., 1998, Antisense Nuc. Acid Drug Dev., 8, 75; Epa et al., 2000, Antisense Nuc. Acid Drug Dev., 10, 469; Broaddus et al., 1998, J. Neurosurg., 88(4), 734; Karle et al., 1997, Eur. J. Pharmocol., 340(2/3), 153; Bannai et al., 1998, Brain Research, 784(1,2), 304; Rajakumar et al., 1997, Synapse, 26(3), 199; Wu-pong et al., 1999, BioPharm, 12(1), 32; Bannai et al., 1998, Brain Res. Protoc., 3(1), 83; and Simantov et al., 1996, Neuroscience, 74(1), 39. Nucleic acid molecules are therefore amenable to delivery to and uptake by cells in the CNS and/or PNS, e.g. neurons, macrophages, white matter axons and endothelial cells.

Delivery systems may include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Non-limiting examples of liposomes which can be used with the compounds of this invention include the following: (1) CellFectin, 1:1.5 (M/M) liposome formulation of the cationic lipid N,NI,NII,NIII-tetramethyl-N,NI,NII,NIII-tetrapalmit-y-spermine and dioleoyl phosphatidylethanolamine (DOPE) (GIBCO BRL); (2) Cytofectin GSV, 2:1 (M/M) liposome formulation of a cationic lipid and DOPE (Glen Research); (3) DOTAP(N-[1-(2,3-dioleoyloxy)-N,N,N-tri-methyl-ammoniummethylsulfate) (Boehringer Manheim); and (4) Lipofectamine, 3:1 (M/M) liposome formulation of the polycationic lipid DOSPA, the neutral lipid DOPE (GIBCO BRL) and Di-Alkylated Amino Acid (DiLA2).

Delivery systems may include patches, tablets, suppositories, pessaries, gels, aqueous and nonaqueous solutions, lotions and creams, and can contain excipients such as solubilizers and enhancers (e.g., propylene glycol, bile salts and amino acids), and other vehicles (e.g., polyethylene glycol, glycerol, fatty acid esters and derivatives, and hydrophilic polymers such as hydroxypropylmethylcellulose and hyaluronic acid).

Nucleic acid molecules may include a bioconjugate, for example a nucleic acid conjugate as described in Vargeese et al., U.S. Ser. No. 10/427,160; U.S. Pat. No. 6,528,631; U.S. Pat. No. 6,335,434; U.S. Pat. No. 6,235,886; U.S. Pat. No. 6,153,737; U.S. Pat. No. 5,214,136; U.S. Pat. No. 5,138,045.

Compositions, methods and kits disclosed herein may include an expression vector that includes a nucleic acid sequence encoding at least one nucleic acid molecule of the invention in a manner that allows expression of the nucleic acid molecule. Methods of introducing nucleic acid molecules or one or more vectors capable of expressing the strands of dsRNA into the environment of the cell will depend on the type of cell and the make up of its environment. The nucleic acid molecule or the vector construct may be directly introduced into the cell (i.e., intracellularly); or introduced extracellularly into a cavity, interstitial space, into the circulation of an organism, introduced orally, or may be introduced by bathing an organism or a cell in a solution containing dsRNA. The cell is preferably a mammalian cell; more preferably a human cell. The nucleic acid molecule of the expression vector can include a sense region and an antisense region. The antisense region can include a sequence complementary to a RNA or DNA sequence encoding HES1, HES5, HEY1, HEY2, ID1, ID2, ID3, CDKN1B, or NOTCH1, and the sense region can include a sequence complementary to the antisense region. The nucleic acid molecule can include two distinct strands having complementary sense and antisense regions. The nucleic acid molecule can include a single strand having complementary sense and antisense regions.

Nucleic acid molecules that interact with target RNA molecules and down-regulate gene encoding target RNA molecules (e.g., HES1, HES5, HEY1, HEY2, ID1, ID2, ID3, CDKN1B, or NOTCH1 mRNA, SEQ ID NO:1-11) may be expressed from transcription units inserted into DNA or RNA vectors. Recombinant vectors can be DNA plasmids or viral vectors. Nucleic acid molecule expressing viral vectors can be constructed based on, but not limited to, adeno-associated virus, retrovirus, adenovirus, or alphavirus. The recombinant vectors capable of expressing the nucleic acid molecules can be delivered as described herein, and persist in target cells. Alternatively, viral vectors can be used that provide for transient expression of nucleic acid molecules. Such vectors can be repeatedly administered as necessary. Once expressed, the nucleic acid molecules bind and down-regulate gene function or expression, e.g., via RNA interference (RNAi). Delivery of nucleic acid molecule expressing vectors can be systemic, such as by intravenous or intramuscular administration, by local administration, by administration to target cells ex-planted from a subject followed by reintroduction into the subject, or by any other means that would allow for introduction into the desired target cell.

Expression vectors may include a nucleic acid sequence encoding at least one nucleic acid molecule disclosed herein, in a manner which allows expression of the nucleic acid molecule. For example, the vector may contain sequence(s) encoding both strands of a nucleic acid molecule that include a duplex. The vector can also contain sequence(s) encoding a single nucleic acid molecule that is self-complementary and thus forms a nucleic acid molecule. Non-limiting examples of such expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725. Expression vectors may also be included in a mammalian (e.g., human) cell.

An expression vector may encode one or both strands of a nucleic acid duplex, or a single self-complementary strand that self hybridizes into a nucleic acid duplex. The nucleic acid sequences encoding nucleic acid molecules can be operably linked in a manner that allows expression of the nucleic acid molecule (see for example Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725).

An expression vector may include one or more of the following: a) a transcription initiation region (e.g., eukaryotic pol I, II or III initiation region); b) a transcription termination region (e.g., eukaryotic pol I, II or III termination region); c) an intron and d) a nucleic acid sequence encoding at least one of the nucleic acid molecules, wherein said sequence is operably linked to the initiation region and the termination region in a manner that allows expression and/or delivery of the nucleic acid molecule. The vector can optionally include an open reading frame (ORF) for a protein operably linked on the 5′-side or the 3′-side of the sequence encoding the nucleic acid molecule; and/or an intron (intervening sequences).

Transcription of the nucleic acid molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L′Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as siNA in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736). The above nucleic acid transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (see Couture and Stinchcomb, 1996 supra).

Nucleic acid molecule may be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45. Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856.

A viral construct packaged into a viral particle would accomplish both efficient introduction of an expression construct into the cell and transcription of dsRNA construct encoded by the expression construct.

Methods for oral introduction include direct mixing of RNA with food of the organism, as well as engineered approaches in which a species that is used as food is engineered to express an RNA, then fed to the organism to be affected. Physical methods may be employed to introduce a nucleic acid molecule solution into the cell. Physical methods of introducing nucleic acids include injection of a solution containing the nucleic acid molecule, bombardment by particles covered by the nucleic acid molecule, soaking the cell or organism in a solution of the RNA, or electroporation of cell membranes in the presence of the nucleic acid molecule. In one embodiment provided herein is a cell comprising a nucleic acid molecule disclosed herein.

Other methods known in the art for introducing nucleic acids to cells may be used, such as chemical mediated transport, such as calcium phosphate, and the like. Thus the nucleic acid molecules may be introduced along with components that perform one or more of the following activities: enhance RNA uptake by the cell, promote annealing of the duplex strands, stabilize the annealed strands, or other-wise increase inhibition/down-regulation of the target gene.

Polymeric nanocapsules or microcapsules facilitate transport and release of the encapsulated or bound dsRNA into the cell. They include polymeric and monomeric materials, especially including polybutylcyanoacrylate. A summary of materials and fabrication methods has been published (see Kreuter, 1991). The polymeric materials which are formed from monomeric and/or oligomeric precursors in the polymerization/nanoparticle generation step, are per se known from the prior art, as are the molecular weights and molecular weight distribution of the polymeric material which a person skilled in the field of manufacturing nanoparticles may suitably select in accordance with the usual skill.

Nucleic acid molecules may be formulated as a microemulsion. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system.

Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO₃₁₀), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

Delivery formulations can include water soluble degradable crosslinked polymers that include one or more degradable crosslinking lipid moiety, one or more PEI moiety, and/or one or more mPEG (methyl ether derivative of PEG (methoxypoly (ethylene glycol)).

### Dosages

The useful dosage to be administered and the particular mode of administration will vary depending upon such factors as the cell type, or for in vivo use, the age, weight and the particular animal and region thereof to be treated, the particular nucleic acid and delivery method used, the therapeutic or diagnostic use contemplated, and the form of the formulation, for example, suspension, emulsion, micelle or liposome, as will be readily apparent to those skilled in the art. Typically, dosage is administered at lower levels and increased until the desired effect is achieved.

The “therapeutically effective dose” for purposes herein is thus determined by such considerations as are known in the art. The dose must be effective to achieve improvement including but not limited to improved survival rate or more rapid recovery, or improvement or elimination of symptoms and other indicators as are selected as appropriate measures by those skilled in the art.

Suitable amounts of nucleic acid molecules may be introduced and these amounts can be empirically determined using standard methods. Effective concentrations of individual nucleic acid molecule species in the environment of a cell may be about 1 femtomolar, about 50 femtomolar, 100 femtomolar, 1 picomolar, 1.5 picomolar, 2.5 picomolar, 5 picomolar, 10 picomolar, 25 picomolar, 50 picomolar, 100 picomolar, 500 picomolar, 1 nanomolar, 2.5 nanomolar, 5 nanomolar, 10 nanomolar, 25 nanomolar, 50 nanomolar, 100 nanomolar, 500 nanomolar, 1 micromolar, 2.5 micromolar, 5 micromolar, 10 micromolar, 100 micromolar or more.

In general, the active dose of nucleic acid compound for humans is in the range of from 1 ng/kg to about 20-100 milligrams per kilogram (mg/kg) body weight of the recipient per day, preferably about 0.01 mg to about 2-10 mg/kg body weight of the recipient per day, in a regimen of a single dose, a one dose per day or twice or three or more times per day for a period of 1-4 weeks or longer. A suitable dosage unit of nucleic acid molecules may be in the range of 0.001 to 0.25 milligrams per kilogram body weight of the recipient per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. Dosage may be from 0.01 ug to 1 g per kg of body weight (e.g., 0.1 ug, 0.25 ug, 0.5 ug, 0.75 ug, 1 ug, 2.5 ug, 5 ug, 10 ug, 25 ug, 50 ug, 100 ug, 250 ug, 500 ug, 1 mg, 2.5 mg, 5 mg, 10 mg, 25 mg, 50 mg, 100 mg, 250 mg, or 500 mg per kg of body weight).

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depends upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

Pharmaceutical compositions that include the nucleic acid molecule disclosed herein may be administered once daily (QD), twice a day (bid), three times a day (tid), four times a day (qid), or at any interval and for any duration that is medically appropriate. However, the therapeutic agent may also be dosed in dosage units containing two, three, four, five, six or more sub-doses administered at appropriate intervals throughout the day. In that case, the nucleic acid molecules contained in each sub-dose may be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. The dosage unit may contain a corresponding multiple of the daily dose. The composition can be compounded in such a way that the sum of the multiple units of a nucleic acid together contain a sufficient dose.

### Pharmaceutical Compositions, Kits, and Containers

Also provided are compositions, kits, containers and formulations that include a nucleic acid molecule (e.g., an siNA molecule) as provided herein for down-regulating expression of HEST, HES5, HEY1, HEY2, ID1, ID2, ID3, CDKN1B, or NOTCH1 for administering or distributing the nucleic acid molecule to a patient. A kit may include at least one container and at least one label. Suitable containers include, for example, bottles, vials, syringes, and test tubes. The containers can be formed from a variety of materials such as glass, metal or plastic. The container can hold amino acid sequence(s), small molecule(s), nucleic acid sequence(s), cell population(s) and/or antibody(s) and/or any other component required for relevant laboratory, prognostic, diagnostic, prophylactic and therapeutic purposes. Indications and/or directions for such uses can be included on or with such container, as can reagents and other compositions or tools used for these purposes.

The container can alternatively hold a composition that is effective for treating, diagnosis, prognosing or prophylaxing a condition and can have a sterile access port (for example the container can be an intravenous solution bag or a vial having a stopper pierceable by a hypodermic injection needle). The active agents in the composition can be a nucleic acid molecule capable of specifically binding HES1, HES5, HEY1, HEY2, ID1, ID2, ID3, CDKN1B, or NOTCH1 mRNA and/or down-regulating the function of HES1, HES5, HEY1, HEY2, ID1, ID2, ID3, CDKN1B, or NOTCH1.

A kit may further include a second container that includes a pharmaceutically-acceptable buffer, such as phosphate-buffered saline, Ringer's solution and/or dextrose solution. It can further include other materials desirable from a commercial and user standpoint, including other buffers, diluents, filters, stirrers, needles, syringes, and/or package inserts with indications and/or instructions for use.

Federal law requires that the use of pharmaceutical compositions in the therapy of humans be approved by an agency of the Federal government. In the United States, enforcement is the responsibility of the Food and Drug Administration, which issues appropriate regulations for securing such approval, detailed in 21 U.S.C. §301-392. Regulation for biologic material, including products made from the tissues of animals is provided under 42 U.S.C. §262. Similar approval is required by most foreign countries. Regulations vary from country to country, but individual procedures are well known to those in the art and the compositions and methods provided herein preferably comply accordingly.

The nucleic acid molecules disclosed herein can be used to treat diseases, conditions or disorders associated with HES1, HES5, HEY1, HEY2, ID1, ID2, ID3, CDKN1B, or NOTCH1, such as disease, injury, condition or pathology in the ear, vestibular sensory system, and any other disease or conditions that are related to or will respond to the levels of HES1, HES5, HEY1, HEY2, ID1, ID2, ID3, CDKN1B, or NOTCH1 in a cell or tissue, alone or in combination with other therapies. As such, compositions, kits and methods disclosed herein may include packaging a nucleic acid molecule disclosed herein that includes a label or package insert. The label may include indications for use of the nucleic acid molecules such as use for treatment or prevention of diseases, disorders, injuries and conditions of the ear or vestibular system, including, without being limited to, Meniere's disease, acoustic trauma, deafness, hearing loss, presbycusis and any other disease or condition disclosed herein. The label may include indications for use of the nucleic acid molecules such as use for treatment or prevention of attenuation of neuronal degeneration. Neuronal degeneration includes for example degeneration of the auditory nerve, (also known as the vestibulocochlear nerve or acoustic nerve and responsible for transmitting sound and equilibrium information from the inner ear to the brain); the hair cells of the inner ear that transmit information to the brain via the auditory nerve, which consists of the cochlear nerve, and the vestibular nerve, and emerges from the medulla oblongata and enters the inner skull via the internal acoustic meatus (or internal auditory meatus) in the temporal bone, along with the facial nerve. The label may include indications for use of the nucleic acid molecules such as use for treatment or prevention of any other disease or conditions that are related to or will respond to the levels of HES1, HES5, HEY1, HEY2, ID1, ID2, ID3, CDKN1B, or NOTCH1 in a cell or tissue, alone or in combination with other therapies. A label may include an indication for use in reducing and/or down-regulating expression of HES1, HES5, HEY1, HEY2, ID1, ID2, ID3, CDKN1B, or NOTCH1. A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

Those skilled in the art will recognize that other treatments, drugs and therapies known in the art can be readily combined with the nucleic acid molecules herein (e.g. dsNA molecules) and are hence contemplated herein.

### Methods of Treatment

In another aspect, the present invention relates to a method for the treatment of a subject in need of treatment for a disease or disorder associated with the abnormal expression of HES1, HES5, HEY1, HEY2, ID1, ID2, ID3, CDKN1B, or NOTCH1, comprising administering to the subject an amount of an inhibitor, which reduces or inhibits expression of HES1, HES5, HEY1, HEY2, ID1, ID2, ID3, CDKN1B, or NOTCH1.

In one embodiment, nucleic acid molecules may be used to down-regulate or inhibit the expression of HES1, HES5, HEY1, HEY2, ID1, ID2, ID3, CDKN1B, or NOTCH1 and/or HES1, HES5, HEY1, HEY2, ID1, ID2, ID3, CDKN1B, or NOTCH1 proteins arising from HES1, HES5, HEY1, HEY2, ID1, ID2, ID3, CDKN1B, or NOTCH1 and/or haplotype polymorphisms that are associated with a disease or condition, (e.g., neurodegeneration). Analysis of HES1, HES5, HEY1, HEY2, ID1, ID2, ID3, CDKN1B, or NOTCH1 and/or HES1, HES5, HEY1, HEY2, ID1, ID2, ID3, CDKN1B, or NOTCH1 genes, and/or protein or RNA levels can be used to identify subjects with such polymorphisms or those subjects who are at risk of developing traits, conditions, or diseases described herein. These subjects are amenable to treatment, for example, treatment with nucleic acid molecules disclosed herein and any other composition useful in treating diseases related to HES1, HES5, HEY1, HEY2, ID1, ID2, ID3, CDKN1B, or NOTCH1 and/or HES1, HES5, HEY1, HEY2, ID1, ID2, ID3, CDKN1B, or NOTCH1 gene expression. As such, analysis of HES1, HES5, HEY1, HEY2, ID1, ID2, ID3, CDKN1B, or NOTCH1 and/or protein or RNA levels can be used to determine treatment type and the course of therapy in treating a subject. Monitoring of protein or RNA levels can be used to predict treatment outcome and to determine the efficacy of compounds and compositions that modulate the level and/or activity of certain genes and/or proteins associated with a trait, condition, or disease.

The invention provides a method of inhibiting the expression of any one of the target genes selected from the group consisting of a gene transcribed into mRNA set forth in any one of SEQ ID NOS:1-11 by at least 40%, preferably by 50%, 60% or 70%, more preferably by 75%, 80% or 90% as compared to a control comprising contacting an mRNA transcript of the target gene of the present invention with one or more of the compounds of the invention.

In one embodiment the oligoribonucleotide inhibits one or more of the target genes disclosed herein, whereby the inhibition is selected from the group comprising inhibition of gene function, inhibition of polypeptide and inhibition of mRNA expression.

In one embodiment the compound inhibits the target polypeptide, whereby the inhibition is selected from the group comprising inhibition of function (which is examined by, for example, an enzymatic assay or a binding assay with a known interactor of the native gene/polypeptide, inter alia), inhibition of protein (which is examined by, for example, Western blotting, ELISA or immuno-precipitation, inter alia) and inhibition of mRNA expression (which is examined by, for example, Northern blotting, quantitative RT-PCR, in-situ hybridization or microarray hybridization, inter alia).

In one embodiment the compound is down-regulating a mammalian polypeptide, whereby the down-regulation is selected from the group comprising down-regulation of function (which is examined by, for example, an enzymatic assay or a binding assay with a known interactor of the native gene/polypeptide, inter alia), down-regulation of protein (which is examined by, for example, Western blotting, ELISA or immuno-precipitation, inter alia) and down-regulation of mRNA expression (which is examined by, for example, Northern blotting, quantitative RT-PCR, in-situ hybridization or microarray hybridization, inter alia).

In additional embodiments the invention provides a method of treating a patient suffering from a disease accompanied by an elevated level of a mammalian gene elected from the group consisting of a gene transcribed into mRNA set forth in any one of SEQ ID NOS:1-11, the method comprising administering to the patient a compound or composition of the invention in a therapeutically effective dose thereby treating the patient.

Methods, molecules and compositions which inhibit a mammalian gene or polypeptide of the present invention are discussed herein at length, and any of said molecules and/or compositions are beneficially employed in the treatment of a patient suffering from any of said conditions. It is to be explicitly understood that known compounds are excluded from the present invention. Novel methods of treatment using known compounds and compositions fall within the scope of the present invention.

The method of the invention includes administering a therapeutically effective amount of one or more compounds which down-regulate expression of a hearing loss associated gene. By “exposure to a toxic agent” is meant that the toxic agent is made available to, or comes into contact with, a mammal. A toxic agent can be toxic to the nervous system. Exposure to a toxic agent can occur by direct administration, e.g., by ingestion or administration of a food, medicinal, or therapeutic agent, e.g., a chemotherapeutic agent, by accidental contamination, or by environmental exposure, e.g., aerial or aqueous exposure.

Further provided is a process of preparing a pharmaceutical composition, which comprises:

providing one or more double stranded molecule disclosed herein; and

admixing said molecule with a pharmaceutically acceptable carrier.

In a preferred embodiment, the molecule used in the preparation of a pharmaceutical composition is admixed with a carrier in a pharmaceutically effective dose. In a particular embodiment the compound of the present invention is conjugated to a steroid or to a lipid or to another suitable molecule e.g. to cholesterol.

Provided are compositions and methods for inhibition of HEST, HES5, HEY1, HEY2, ID1, ID2, ID3, CDKN1B, or NOTCH1 expression by using small nucleic acid molecules as provided herein, such as short interfering nucleic acid (siNA), interfering RNA (RNAi), short interfering RNA (siRNA), double-stranded RNA (dsRNA), micro-RNA (miRNA), and short hairpin RNA (shRNA) molecules capable of down-regulating HES1, HES5, HEY1, HEY2, ID1, ID2, ID3, CDKN1B, or NOTCH1 gene expression, or of mediating RNA interference against HES1, HES5, HEY1, HEY2, ID1, ID2, ID3, CDKN1B, or NOTCH1 gene expression. The composition and methods disclosed herein are also useful in treating various conditions or diseases, such as, e.g. ear and vestibular sensory system disorders, disease and injury.

The nucleic acid molecules disclosed herein individually, or in combination or in conjunction with other drugs, can be used for preventing or treating diseases, traits, conditions and/or disorders associated with HES1, HES5, HEY1, HEY2, ID1, ID2, ID3, CDKN1B, or NOTCH1, such as diseases, disorders and injuries described herein.

The nucleic acid molecules disclosed herein are able to down-regulate the expression of HES1, HES5, HEY1, HEY2, ID1, ID2, ID3, CDKN1B, or NOTCH1 in a sequence specific manner. The nucleic acid molecules may include a sense strand and an antisense strand which include contiguous nucleotides that are at least partially complementary (antisense) to a portion of HES1, HES5, HEY1, HEY2, ID1, ID2, ID3, CDKN1B, or NOTCH1 mRNA.

In some embodiments, dsRNA specific for HES1, HES5, HEY1, HEY2, ID1, ID2, ID3, CDKN1B, or NOTCH1 can be used in conjunction with other therapeutic agents and/or dsRNA specific for other molecular targets, such as, without being limited to various proapoptotic genes.

A method for treating or preventing HES1, HES5, HEY1, HEY2, ID1, ID2, ID3. CDKN1B, or NOTCH1 associated disease or condition in a subject or organism may include contacting the subject or organism with a nucleic acid molecule as provided herein under conditions suitable to down-regulate the expression of the gene in the subject or organism.

A method for treating or preventing an ear disorder in a subject or organism may include contacting the subject or organism with a nucleic acid molecule under conditions suitable to down-regulate the expression of the HES1, HES5, HEY1, HEY2, ID1, ID2, ID3, CDKN1B, or NOTCH1 gene in the subject or organism.

In preferred embodiments the subject being treated is a warm-blooded animal and, in particular, mammals including human.

The methods disclosed herein comprise administering to the subject one or more inhibitory compounds which down-regulate expression of HES1, HES5, HEY1, HEY2, ID1, ID2, ID3, CDKN1B, or NOTCH1; and in particular siRNA in a therapeutically effective dose so as to thereby treat the subject.

The molecules disclosed herein down-regulate the expression of HES1, HES5, HEY1, HEY2, ID1, ID2, ID3, CDKN1B, or NOTCH1, particularly to novel double stranded RNA compounds (dsRNAs), in the treatment of diseases or conditions in which down-regulation of the expression of HES1, HES5, HEY1, HEY2, ID1, ID2, ID3, CDKN1B, or NOTCH1 is beneficial.

Methods, molecules and compositions which down-regulate HES1, HES5, HEY1, HEY2, ID1, ID2, ID3, CDKN1B, or NOTCH1 are discussed herein at length, and any of said molecules and/or compositions may be beneficially employed in the treatment of a subject suffering from any of said conditions. Sense strand and antisense strand oligonucleotide sequences useful in generating dsRNA are set forth in SEQ ID NOS:23-26912. Preferred oligonucleotide sequences useful in the preparation of dsRNA that down-regulate expression of HES1 are set forth in SEQ ID NOS:26667-26690 and 26691-26706; of HES5 are set forth in SEQ ID NOS:26707-26724 and 26725-26732; of HEY1 are set forth in SEQ ID NOS:26733-26760 and 26761-2677; of HEY2 are set forth in SEQ ID NOS:26779-26784 and 26785-26788; of ID1 are set forth in SEQ ID NOS:26789-26808 and 26809-26816; of ID2 are set forth in SEQ ID NOS:26817-26824 and 26825-26832; of ID3 are set forth in SEQ ID NOS:26833-26850 and 26851-26866; of CDKN1B are set forth in SEQ ID NOS:26867-26886 and 26887-26900 or NOTCH1 are set forth in SEQ ID NOS:26901-26910 and 26911-26912. In preferred embodiments the subject being treated is a warm-blooded animal and, in particular, mammals including human.

The methods disclosed herein comprise administering to the subject one or more inhibitory compounds which down-regulate expression of the genes of Table 1A (SEQ ID NOS:1-11); and in particular siRNA in a therapeutically effective dose so as to thereby treat the subject.

“Treatment” refers to both therapeutic treatment and prophylactic or preventative measures, wherein the object is to prevent a disorder or reduce the symptoms of a disorder, such as hearing disorder or impairment (or balance impairment), or to prevent or reduce cell death associated with a hearing loss-associated disease as listed herein. Those in need of treatment include those already experiencing the disease or condition, those prone to having the disease or condition, and those in which the disease or condition is to be prevented. The compounds of the invention are administered before, during or subsequent to the onset of the disease or condition.

Without being bound by theory, the hearing impairment may be due to apoptotic inner ear hair cell damage or loss, wherein the damage or loss is caused by infection, mechanical injury, loud sound, aging, or, in particular, chemical-induced ototoxicity. Ototoxins include therapeutic drugs including antineoplastic agents, salicylates, quinines, and aminoglycoside antibiotics, contaminants in foods or medicinals, and environmental or industrial pollutants. Typically, treatment is performed to prevent or reduce ototoxicity, especially resulting from or expected to result from administration of therapeutic drugs. Preferably a therapeutically effective composition is given immediately after the exposure to prevent or reduce the ototoxic effect. More preferably, treatment is provided prophylactically, either by administration of the composition prior to or concomitantly with the ototoxic pharmaceutical or the exposure to the ototoxin.

By “ototoxin” in the context of the present invention is meant a substance that through its chemical action injures, impairs or inhibits the activity of the sound receptors component of the nervous system related to hearing, which in turn impairs hearing (and/or balance). In the context of the present invention, ototoxicity includes a deleterious effect on the inner ear hair cells. Ototoxic agents that cause hearing impairments include, but are not limited to, neoplastic agents such as vincristine, vinblastine, cisplatin and cisplatin-like compounds, taxol and taxol-like compounds, dideoxy-compounds, e.g., dideoxyinosine; alcohol; metals; industrial toxins involved in occupational or environmental exposure; contaminants of food or medicinals; and over-doses of vitamins or therapeutic drugs, e.g., antibiotics such as penicillin or chloramphenicol, and megadoses of vitamins A, D, or B6, salicylates, quinines and loop diuretics. By “exposure to an ototoxic agent” is meant that the ototoxic agent is made available to, or comes into contact with, a mammal. Exposure to an ototoxic agent can occur by direct administration, e.g., by ingestion or administration of a food, medicinal, or therapeutic agent, e.g., a chemotherapeutic agent, by accidental contamination, or by environmental exposure, e.g., aerial or aqueous exposure.

Hearing impairment relevant to the invention may be due to end-organ lesions involving inner ear hair cells, e.g., acoustic trauma, viral endolymphatic labyrinthitis, Meniere's disease. Hearing impairments include tinnitus, which is a perception of sound in the absence of an acoustic stimulus, and may be intermittent or continuous, wherein there is diagnosed a sensorineural loss. Hearing loss may be due to bacterial or viral infection, such as in herpes zoster oticus, purulent labyrinthitis arising from acute otitis media, purulent meningitis, Chronic otitis media, sudden deafness including that of viral origin, e.g., viral endolymphatic labyrinthitis caused by viruses including mumps, measles, influenza, chicken pox, mononucleosis and adenoviruses. The hearing loss can be congenital, such as that caused by rubella, anoxia during birth, bleeding into the inner ear due to trauma during delivery, ototoxic drugs administered to the mother, erythroblastosis fetalis, and hereditary conditions including Waardenburg's syndrome and Hurler's syndrome.

The hearing loss can be noise-induced, generally due to a noise greater than 85 decibels (db) that damages the inner ear. In a particular aspect, the hearing loss is caused by an ototoxic drug that effects the auditory portion of the inner ear, particularly inner ear hair cells. Incorporated herein by reference are chapters 196, 197, 198 and 199 of The Merck Manual of Diagnosis and Therapy, 14th Edition, (1982), Merck Sharp & Dome Research Laboratories, N.J. and corresponding chapters in the most recent 16th edition, including Chapters 207 and 210) relating to description and diagnosis of hearing and balance impairments.

In one embodiment, provided is a method for treating a mammal having or prone to a hearing (or balance) impairment or treating a mammal prophylactically in conditions where inhibition of the genes of the invention is beneficial. The method would prevent or reduce the occurrence or severity of a hearing (or balance) impairment that would result from inner ear cell injury, loss, or degeneration, in particular caused by an ototoxic agent. The method includes administering a therapeutically effective amount of one or more compounds which down-regulate expression of HEST, HES5, HEY1, HEY2, ID1, ID2, ID3, CDKN1B, or NOTCH1, particularly the novel siRNAs of the present invention.

It is the object to provide a method and compositions for treating a mammal, to prevent, reduce, or treat a hearing impairment, disorder or imbalance, optionally an ototoxin-induced hearing condition, by administering to a mammal in need of such treatment a composition of the invention. One embodiment of the invention is a method for treating a hearing disorder or impairment wherein the ototoxicity results from administration of a therapeutically effective amount of an ototoxic pharmaceutical drug. Typical ototoxic drugs are chemotherapeutic agents, e.g. antineoplastic agents, and antibiotics. Other possible candidates include loop-diuretics, quinines or a quinine-like compound, and salicylate or salicylate-like compounds.

The methods and compositions disclosed herein are also effective when the ototoxic compound is an antibiotic, preferably an aminoglycoside antibiotic. Ototoxic aminoglycoside antibiotics include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. Particular antibiotics include neomycin B, kanamycin A, kanamycin B, gentamicin C1, gentamicin C1a, and gentamicin C2. The methods and compositions are also effective when the ototoxic compound is a neoplastic agent such as vincristine, vinblastine, cisplatin and cisplatin-like compounds and taxol and taxol-like compounds. The methods and compositions are also effective in the treatment of acoustic trauma or mechanical trauma, preferably acoustic or mechanical trauma that leads to inner ear hair cell loss or outer ear hair cell loss. Acoustic trauma to be treated in the present invention may be caused by a single exposure to an extremely loud sound of above 120-140 decibels, or following long-term exposure to everyday loud sounds above 85 decibels. The compositions of the present invention are also effective as a preventive treatment in patients expecting an acoustic trauma. Mechanical inner ear trauma to be treated in the present invention is for example the inner ear trauma following an operation for insertion of an electronic device in the inner ear. The molecules and compositions disclosed herein prevent or minimize the damage to inner ear hair cells associated with this operation.

In some embodiments the molecules and compositions provided herein are co-administered with an ototoxin. For example, an improved method is provided for treatment of infection of a mammal by administration of an aminoglycoside antibiotic, the improvement comprising administering a therapeutically effective amount of one or more compounds (particularly novel siRNAs) which down-regulate expression of HES1, HES5, HEY1, HEY2, ID1, ID2, ID3, CDKN1B, or NOTCH1, to the patient in need of such treatment to reduce or prevent ototoxin-induced hearing impairment associated with the antibiotic. The compounds which down-regulate expression of HES1, HES5, HEY1, HEY2, ID1, ID2, ID3, CDKN1B, or NOTCH1, particularly novel siRNAs are preferably administered locally within the inner ear.

In yet another embodiment an improved method for treatment of cancer in a mammal by administration of a chemotherapeutic compound is provided, wherein the improvement comprises administering a therapeutically effective amount of a composition of the invention to the patient in need of such treatment to reduce or prevent ototoxin-induced hearing impairment associated with the chemotherapeutic drug. The compounds which reduce or prevent the ototoxin-induced hearing impairment, e.g. the dsRNA molecules disclosed herein, inter alia are preferably administered directly to the cochlea as naked dsRNA in a vehicle such as PBS or other physiological solutions, but may alternatively be administered with a delivery vehicle as described above.

In another embodiment the methods of treatment are applied to treatment of hearing impairment resulting from the administration of a chemotherapeutic agent in order to treat its ototoxic side-effect. Ototoxic chemotherapeutic agents amenable to the methods of the invention include, but are not limited to an antineoplastic agent, including cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas. Cisplatin-like compounds include carboplatin (Paraplatin®), tetraplatin, oxaliplatin, aroplatin and transplatin inter alia.

In another embodiment the methods are applied to hearing impairments resulting from the administration of quinine and its synthetic substitutes, typically used in the treatment of malaria, to treat its ototoxic side-effect.

In another embodiment the methods are applied to hearing impairments resulting from administration of a diuretic to treat its ototoxic side-effect. Diuretics, particularly “loop” diuretics, i.e. those that act primarily in the Loop of Henle, are candidate ototoxins. Illustrative examples, not limited to the invention method, include furosemide, ethacrylic acid, and mercurials. Diuretics are typically used to prevent or eliminate edema. Diuretics are also used in nonedematous states for example hypertension, hypercalcemia, idiopathic hypercalciuria, and nephrogenic diabetes insipidus.

In some embodiments combination therapy is preferred. Combination therapy is achieved by administering two or more agents (i.e. two or more dsRNA or at least one dsRNA and at least one another therapeutic agent) each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within one or several hours of each other or within one or several days of each other or within several weeks of each other. In some cases even longer intervals are possible. The two or more agents used in combination therapy may or may not be present within the patient's body at the same time. Combination therapy includes two or more administrations of one or more of the agents used in the combination. For example, if dsRNA1 and dsRNA2 (i.e. wherein dsRNA1 targets gene 1 and dsRNA2 targets gene 2) are used in a combination, one could administer them sequentially in any combination one or more times, e.g., in the order dsRNA1-dsRNA2, dsRNA2-dsRNA1, dsRNA1-dsRNA2-dsRNA1, dsRNA2-dsRNA1-dsRNA2, dsRNA1-dsRNA1-dsRNA2, dsRNA1-dsRNA2-dsRNA2 etc.

### Hearing Regeneration

Sensory progenitor cells can develop as either hair cells or supporting cells. Ablation studies indicate that removal of a hair cell changes the fate of a surrounding cell from a supporting to a hair cell. This response suggests that hair cells generate inhibitory signals that prevent neighboring cells from developing as hair cells. This type of interaction is consistent with the effects of Notch-mediated lateral inhibition. Consistent with this hypothesis, two Notch ligands, Jag2 and delta 1 (Dll1) are rapidly upregulated in a subset of Atoh1-positive cells. The expression of these ligands leads to activation of Notch 1 and the increased transcription of two Notch pathway target genes, Hes1 and Hes5 in cells that will develop as supporting cells. Deletion of any of the genes in this pathway leads to an overproduction of hair cells, suggesting that Notch signalling has a role in diverting progenitor cells from the hair cell fate. The mechanism of this diversion has been examined using cells in Kolliker's organ. First, co-transfection of Kolliker's organ cells with Atoh1 and Hes1 was sufficient to inhibit hair cell formation, suggesting that Atoh1 transcription is a target of HES1 in the ear. Second, transient activation of ATOH1 in patches of Kolliker's organ cells leads to activation of the Notch signalling pathway within those cells and to the inhibition of ATOH1 and hair cell fate in a subset of those cells.

Mitotic regeneration has never been shown in the postnatal mammalian cochlea. However, recent results indicate that the forced expression of Math1 (Atoh1) in cells of the inner ear of mammals can lead to their differentiation into hair cells. Unlike regeneration in birds, this differentiation appears to occur without concomitant supporting cell proliferation. Effective therapeutic strategies in humans are likely to require the generation of new supporting cells as well as new hair cells, so it is important to test the ability of mammalian supporting cells to divide and differentiate at postnatal times. White et al demonstrate that prospectively identified, post-mitotic, postnatal supporting cells are capable of proliferation and subsequent trans-differentiation into hair cells. In addition, the age-dependent change in the ability of supporting cells to down-regulate p27^{Kip1} suggests a mechanistic explanation for the failure of supporting cell proliferation in response to the loss of hair cells, both in culture and in vivo.

Coordination of cell cycle exit and cellular differentiation has a key role in cell and tissue patterning. In some systems, the activity of bHLH genes such as Atoh1 must be actively inhibited to prevent premature differentiation. One family of factors that has a role in this inhibition is the Id (inhibitors of differentiation) family. These are HLH molecules that inhibit bHLH activity through competition for a common dimer partner, E47. Prior to hair cell formation, three Id genes—Id1, Id2 and Id3—are broadly expressed throughout the floor of the cochlear duct including the domain of Atoh1 expression. As development continues, Id expression is specifically down regulated in developing hair cells, suggesting that loss of Id function relieves an inhibition of Atoh1 activity in those cells. Consistent with this hypothesis, prolonged expression of Id3 in sensory progenitors inhibits hair cell formation, suggesting that down regulation of the Id family is a key step in hair cell development.

Zine et al. (J. Neurosci. 2001 21(13):4712-20.) demonstrate that Hes1 and Hes5 activities are important for repressing the commitment of progenitor cells to IHCs and OHCs fates, respectively, likely by antagonizing Math1. This negative regulation is critical for the correct number of hair cells to be produced and for the establishment of the normal cochlear mosaic of a single row of IHCs and three rows of OHCs. In the vestibular system, Hes1 and Hes5 also act as negative regulators of hair cell differentiation within the utricle and saccule epithelia. It is possible that simultaneous down-regulation of both of Hes1 and Hes5 in the cochlea might be used to stimulate the replacement of lost auditory hair cells. Such studies may have a significant therapeutic value, because loss of auditory hair cells through disease, trauma, and aging is a common cause of hearing loss and/or deafness.

Details of certain indications in which the compounds disclosed herein are useful as therapeutics are described herein.

The invention has been described in an illustrative manner, and it is to be understood that the terminology used is intended to be in the nature of words of description rather than of limitation.

Obviously, many modifications and variations are possible in light of the above teachings. It is, therefore, to be understood that within the scope of the appended claims, the invention can be practiced otherwise than as specifically described.

Throughout this application, various publications, including United States Patents, are referenced by author and year and patents by number. The disclosures of these publications and patents and patent applications in their entireties are hereby incorporated by reference into this application in order to more fully describe the state of the art to which this invention pertains.

The present invention is illustrated in detail below with reference to examples, but is not to be construed as being limited thereto.

Citation of any document herein is not intended as an admission that such document is pertinent prior art, or considered material to the patentability of any claim of the present invention. Any statement as to content or a date of any document is based on the information available to applicant at the time of filing and does not constitute an admission as to the correctness of such a statement.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out as in standard PCR Protocols: A Guide To Methods And Applications, Academic Press, San Diego, Calif. (1990). In situ PCR in combination with Flow Cytometry (FACS) can be used for detection of cells containing specific DNA and mRNA sequences (Testoni et al., Blood 1996, 87:3822.) Methods of performing RT-PCR are well known in the art.

### Example 1

### In Vitro Testing of dsRNA Molecules

About 1.5−2×10⁵ tested cells (HeLa cells and/or 293T cells for siRNA targeting human genes and NRK52 (normal rat kidney proximal tubule cells) cells and/or NMuMG cells (mouse mammary epithelial cell line) for siRNA targeting the rat/mouse gene) were seeded per well in 6 wells plate (70-80% confluent).

24 hours later, cells were transfected with dsRNA molecules using the Lipofectamine™ 2000 reagent (Invitrogen) at final concentrations of 5 nM or 20 nM. The cells were incubated at 37° C. in a CO₂ incubator for 72 h.

As positive control for transfection, PTEN-Cy3 labeled dsRNA molecules were used. GFP dsRNA molecules were used as negative control for siRNA activity.

At 72 h after transfection, cells were harvested and RNA was extracted from cells. Transfection efficiency was tested by fluorescent microscopy.

The percent of inhibition of gene expression using specific preferred siRNA structures was determined using qPCR analysis of a target gene in cells expressing the endogenous gene.

### Body Fluid/Cell Stability Assay

The modified compounds disclosed herein are tested for duplex stability in human, rat or mouse plasma or human, rat or mouse serum (to test in model system), or CSF (cerebrospinal fluid; human, mouse or rat) or human cell extract, as follows:

For example: dsRNA molecules at final concentration of 7 uM are incubated at 37° C. in 100% human serum (Sigma Cat#H4522). (siRNA stock 100 uM diluted in human serum 1:14.29 or human tissue extract from various tissue types.). Five ul (5 ul) are added to 15 ul 1.5×TBE-loading buffer at different time points (for example 0, 30 min, 1 h, 3 h, 6 h, 8 h, 10 h, 16 h and 24 h). Samples are immediately frozen in liquid nitrogen and are kept at −20° C.

Each sample is loaded onto a non-denaturing 20% acrylamide gel, prepared according to methods known in the art. The oligos are visualized with ethidium bromide under UV light.

### Exonuclease Stability Assay

To study the stabilization effect of 3′ non-nucleotide moieties on a nucleic acid molecule the sense strand, the antisense strand and the annealed dsRNA duplex are incubated in cytosolic extracts prepared from different cell types.

Extract: HCT116 cytosolic extract (12 mg/ml).

Extract buffer: 25 mM Hepes pH-7.3 at 37oC; 8 mM MgCl; 150 mM NaCl with 1 mM DTT was added fresh immediately before use.

Method: 3.5 ml of test dsRNA (100 mM), were mixed with 46.5 ml contain 120 mg of HCT116 cytosolic extract. The 46.5 ml consists of 12 ml of HCT116 extract, and 34.5 ml of the extract buffer supplemented with DTT and protease inhibitors cocktail/100 (Calbiochem, setIII-539134). The final concentration of the siRNA in the incubation tube is 7 mM. The sample is incubated at 37oC, and at the indicated time point 5 ml are moved to fresh tube, mixed with 15 ml of 1×TBE-50% Glycerol loading buffer, and snap frozen in Liquid N2. The final concentration of the siRNA in the loading buffer is 1.75 mM (21 ng siRNA/ml). For analyses by native PAGE and EtBr staining 50 ng are loaded per lane. For Northern analyses 1 ng of tested siRNA are loaded per lane.

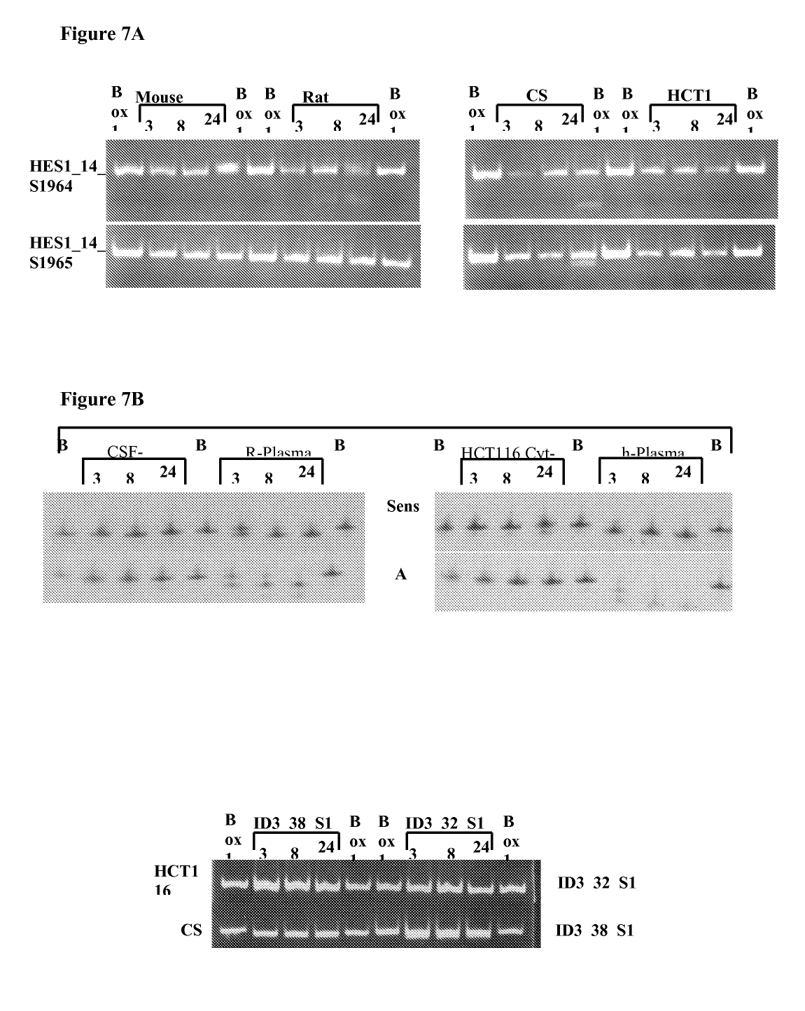

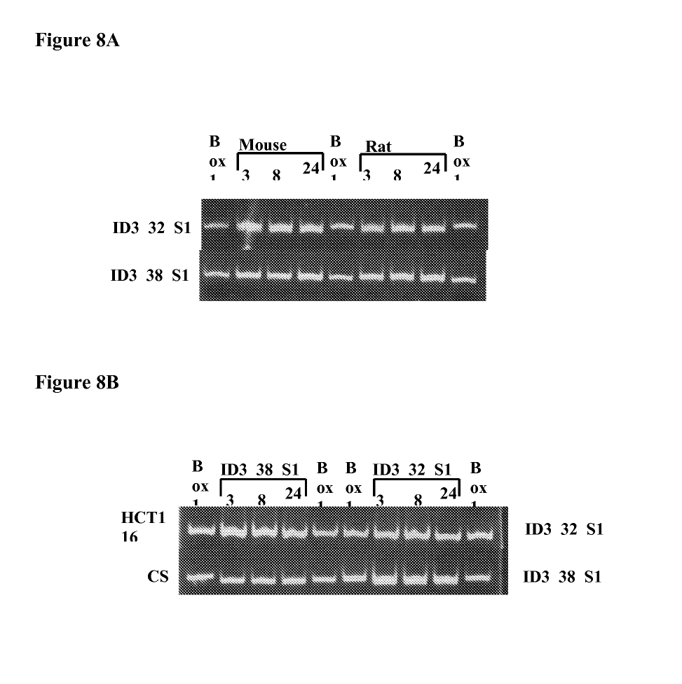

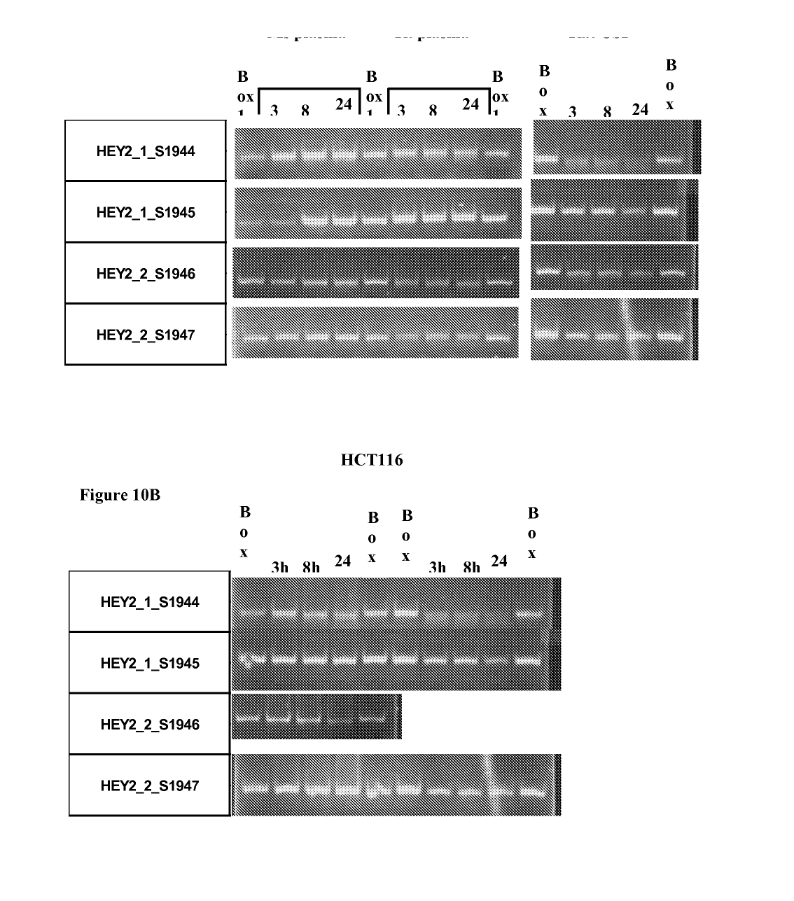

FIGS. 7A, 7B, 8A, 8B, 9A, 10A and 10B show plasma and or cell extract stability of dsRNA disclosed herein.

Innate Immune Response to dsRNA Molecules:

Fresh human blood (at RT) is mixed at 1:1 ratio with sterile 0.9% NaCl at RT, and gently loaded (1:2 ratio) on Ficoll (Lymphoprep, Axis-Shield cat# 1114547). Samples are centrifuged at RT (22OC, 800 g) in a swinging centrifuge for 30 minutes, washed with RPMI1640 medium and centrifuged (RT, 250 g) for 10 minutes. Cells are counted and seeded at final concentration of 1.5×106 cell/ml in growth medium (RPMI1640+10% FBS+2 mM L-glutamine+1% Pen-Strep) and incubated for 1 hour at 37° C. before dsRNA treatment. Cells are exposed to the test dsRNAs at different concentrations using the Lipofectamine™2000 reagent (Invitrogen) according to manufacturer's instructions and incubated at 37° C. in a 5% CO₂ incubator for 24 hours.

As a positive control for IFN response, cells are treated with either poly(I:C), a synthetic analog of double strand RNA (dsRNA) which is a TLR3 ligand (InvivoGen Cat# tlrl-pic) at final concentrations of 0.25-5.0 μg/mL or to Thiazolaquinolone (CL075), a TLR 7/8 ligand (InvivoGen Cat# tlrl-c75) at final concentrations of 0.075-2 μg/mL. Cell treated with Lipofectamine™2000 reagent are used as negative (reference) control for IFN response.

At about 24 hours following incubation, cells are collected and supernatant is transferred to new tubes. Samples are frozen immediately in liquid nitrogen and secretion of IL-6 and TNF-α cytokines was tested using IL-6, DuoSet ELISA kit (R&D System DY2060), and TNF-α, DuoSet ELISA kit (R&D System DY210), according to manufacturer's instructions. RNA is extracted from the cell pellets and mRNA levels of human genes IFIT1 (interferon-induced protein with tetratricopeptide repeats 1) and MX1 (myxovirus (influenza virus) resistance 1, interferon-inducible protein p78) were measured by qPCR. Measured mRNA quantities are normalized to the mRNA quantity of the reference gene peptidylprolyl isomerase A (cyclophilin A; CycloA). Induction of IFN-signaling is evaluated by comparing the quantity of mRNA from IFIT1 and MX1 genes from treated cells, relative to their quantities non-treated cells. The qPCR results are those that passed QC standards, i.e. the value of the standard curve slope was in the interval [−4, −3], R2 >0.99, no primer dimers. Results that do not pass the QC requirements are disqualified from analysis.

In general, the dsRNAs having specific sequences that were selected for in vitro testing were specific for human and a second species such as rat or rabbit genes. The dsRNA were tested for activity to Human (Hu), mouse (Ms), rat (Rt), chinchilla (Chn) and or guinea-pig (GP) target gene. For example, activity in chinchilla was tested by cloning the chinchilla target gene (i.e. CDKN1B) and expressing in a 293 or HeLa cell line. Similar results are obtained using siRNAs having these RNA sequences and modified as described herein.

Table B below shows modification patterns of dsRNA nucleic acid and summarizes the in vitro results obtained for some of the nucleic acid molecules with various modifications. The siRNAs used in the experiments are all 19-mers. The in-vitro activity in Table B is demonstrated as the % residual target mRNA relative to control. The S500 dsRNA molecules have the following modification: Alternating 2′-O-methyl (Me) sugar modified ribonucleotides are present in the first, third, fifth, seventh, ninth, eleventh, thirteenth, fifteenth, seventeenth and nineteenth positions of the antisense strand, whereby the very same modification, i.e. a 2′-O-Me sugar modified ribonucleotides are present in the second, fourth, sixth, eighth, tenth, twelfth, fourteenth, sixteenth and eighteenth positions of the sense strand.

The S129 AND s505 dsRNA molecules have the following modifications: 2′-O-methyl sugar modified ribonucleotides in positions 1,3,5,7,9,11,13,15,17,19 (_S129) or in positions 2,4,6,8,11,13,15,17,19 (_S505) and a sense strand with the following modifications: L-DNA nucleotide at position 18; a DNA nucleotide at position 15 (_S215), and optionally inverted abasic nucleotide, a mirror nucleotide or C6-amine phosphate at the 5′ end.

TABLE B

| | | | activity at 20 nM (% residual target | | | | |
|---|---|---|---|---|---|---|---|
| | | Antisense strand | mRNA relative to control) | | | | |
| Name | Sense strand 5 -> 3 | 5 -> 3 | HUM | Rt | Ms | Chn | GP |
| CDKN1B_1_S500 | rC; mC; rA; mU; rA; mU; rU; | mU; rU; mU; rU; mA; rG; | 63, | | | | |
| | mG; rG; mG; rC; mC; rA; | mU; rG; mG; rC; mC; rC; | 100 | | | | |
| | mC; rU; mA; rA; mA; rA | mA; rA; mU; rA; mU; | | | | | |
| | | rG; mG | | | | | |
| CDKN1B_2_S500 | rG; mG; rU; mG; rC; mU; rU; | mU; rU; mC; rA; mA; rA; | 70, 86 | | | | |
| | mG; rG; mG; rA; mG; rU; | mA; rC; mU; rC; mC; rC; | | | | | |
| | mU; rU; mU; rG; mA; rA | mA; rA; mG; rC; mA; | | | | | |
| | | rC; mC | | | | | |
| CDKN1B_3_S500 | rC; mG; rC; mA; rU; mU; rU; | mU; rU; mU; rG; mG; rG; | 36, 29, | | | | |
| | mG; rG; mU; rG; mG; rA; | mU; rC; mC; rA; mC; rC; | 25 | | | | |
| | mC; rC; mC; rA; mA; rA | mA; rA; mA; rU; mG; | | | | | |
| | | rC; mG | | | | | |
| CDKN1B_4_S500 | rG; mC; rA; mA; rU; mU; rA; | mU; rA; mA; rG; mG; rA; | 29, 25, | 27, 11, | | 43, 36, 14, | 29, 50, |
| | mG; rG; mU; rU; mU; rU; | mA; rA; mA; rA; mC; rC; | 41, 54 | 26 | | 17 | 24, 28 |
| | mU; rC; mC; rU; mU; rA | mU; rA; mA; rU; mU; | | | | | |
| | | rG; mC | | | | | |
| CDKN1B_5_S500 | rG; mC; rU; mG; rA; mU; rA; | mU; rU; mU; rU; mA; rA; | 29, 100 | | | | |
| | mC; rU; mU; rC; mA; rU; | mA; rU; mG; rA; mA; rG; | | | | | |
| | mU; rU; mA; rA; mA; rA | mU; rA; mU; rC; mA; | | | | | |
| | | rG; mC | | | | | |
| CDKN1B_6_S500 | rC; mA; rC; mU; rG; mA; rA; | mU; rU; mA; rG; mU; rA; | 41 | | | | |
| | mA; rA; mA; rU; mU; rA; | mU; rA; mA; rU; mU; rU; | | | | | |
| | mU; rA; mC; rU; mA; rA | mU; rU; mC; rA; mG; | | | | | |
| | | rU; mG | | | | | |
| CDKN1B_7_S500 | rC; mG; rA; mA; rG; mA; rC; | mU; rU; mU; rA; mC; rG; | | | 59, 22 | 45 | |
| | mG; rU; mC; rA; mA; rA; | mU; rU; mU; rG; mA; rC; | | | | | |
| | mC; rG; mU; rA; mA; rA | mG; rU; mC; rU; mU; | | | | | |
| | | rC; mG | | | | | |
| CDKN1B_8_S500 | rC; mC; rG; mA; rA; mG; rA; | mU; rU; mA; rC; mG; rU; | | | | 61 | 47 |
| | mC; rG; mU; rC; mA; rA; | mU; rU; mG; rA; mC; rG; | | | | | |
| | mA; rC; mG; rU; mA; rA | mU; rC; mU; rU; mC; | | | | | |
| | | rG; mG | | | | | |
| CDKN1B_9_S500 | rG; mG; rA; mA; rU; mU; rU; | mU; rU; mC; rU; mG; rA; | 5, 31, 28, | | | 47, 15, | 38, |
| | mC; rG; mA; rU; mU; rU; | mA; rA; mA; rU; mC; rG; | 48 | | | 23 | 49, |
| | mU; rC; mA; rG; mA; rA | mA; rA; mA; rU; mU; | | | | | 23 |
| | | rC; mC | | | | | |
| CDKN1B_10_S500 | rC; mA; rU; mU; rG; mU; rA; | mA; rU; mA; rC; mA; rC; | 37, | 30, 34, | | 45, 16, | 29, 37, |
| | mC; rU; mA; rC; mC; rU; | mA; rG; mG; rU; mA; rG; | 70, 20 | 44 | | 17 | 32 |
| | mG; rU; mG; rU; mA; rU | mU; rA; mC; rA; mA; | | | | | |
| | | rU; mG | | | | | |
| CDKN1B_11_S500 | rG; mG; rU; mU; rU; mU; rU; | mA; rA; mG; rC; mA; rA; | 33, 18, | 12, 17 | | 100, | 41, 28 |
| | mC; rC; mU; rU; mA; rU; | mA; rU; mA; rA; mG; rG; | 60 | | | 11, 13 | |
| | mU; rU; mG; rC; mU; rU | mA; rA; mA; rA; mA; | | | | | |
| | | rC; mC | | | | | |
| CDKN1B_4_S2018 | rG; mC; rA; rA; rU; mU; rA; | mU; rA; rA; rG; rG; rA; | | | | | |
| | rG; rG; rU; rU; rU; rU; | rA2p; rA; rA; rA; rC; | | | | | |
| | rU; mC; rC; rU; mU; rA; | rC; mU; rA; rA; rU; mU; | | | | | |
| | zc3p | rG; rC; zc3p; zc3p | | | | | |
| | | $ | | | | | |
| CDKN1B_4_S2019 | rG; mC; rA; rA; rU; mU; rA; | mU; rA; rA; rG; rG; rA; | | | | | |
| | rG; rG; rU; mU; mU; mU; | rA2p; rA; rA; rA; rC; | | | | | |
| | rU; rC; mC; rU; mU; rA; | rC; mU; rA; rA; rU; mU; | | | | | |
| | zc3p | rG; rC; zc3p; zc3p | | | | | |
| | | $ | | | | | |
| CDKN1B_4_S2075 | zidB; rG; mC; rA; rA; rU; | mU; rA; rA; rG; rG; rA; | | | | | |
| | mU; rA; rG; rG; rU; rU; | rA2p; rA; rA; rA; rC; | | | | | |
| | rU; rU; rU; mC; rC; rU; mU; | rC; mU; rA; rA; rU; mU; | | | | | |
| | rA; zc3p | rG; rC; zc3p; zc3p | | | | | |
| | | $ | | | | | |
| CDKN1B_4_S2076 | zc6Np; rG; mC; rA; rA; rU; | mU; rA; rA; rG; rG; rA; | | | | | |
| | mU; rA; rG; rG; rU; rU; | rA2p; rA; rA; rA; rC; | | | | | |
| | rU; rU; rU; mC; rC; rU; | rC; mU; rA; rA; rU; mU; | | | | | |
| | mU; rA; zc3p | rG; rC; zc3p; zc3p | | | | | |
| | | $ | | | | | |
| CDKN1B_31_S2020 | mC; rA; rG; rC; rG; rC; rA; | mU; rG; rA; rA; rA; rU; | | | | | |
| | rA; rG; rU; rG; rG; rA; | rU2p; rC; mC; rA; mC; | | | | | |
| | rA; rU2p; rU2p; rU2p; | rU; mU; rG; mC; rG; rC; | | | | | |
| | rC2p; rA2p; zc3p | mU; rG; zc3p; zc3p | | | | | |
| | | $ | | | | | |
| CDKN1B_31_S2021 | mC; rA; rG; rC; rG; rC; rA; | mU; rG; rA; rA; rA; rU; | | | | | |
| | rA; rG; mU; rG; rG; rA; | rU2p; rC; mC; rA; mC; | | | | | |
| | rA; rU; rU; rU; mC; rA; | rU; mU; rG; mC; rG; rC; | | | | | |
| | zc3p | mU; rG; zc3p; zc3p | | | | | |
| | | $ | | | | | |
| CDKN1B_31_S2022 | mC; rA; rG; rC; rG; rC; rA; | mU; rG; rA; rA; rA; rU; | | | | | |
| | rA; rG; rU; rG; rG; rA; | rU2p; rC; mC; rA; rC; | | | | | |
| | rA; rU2p; rU2p; rU2p; | rU; mU; rG; mC; rG; rC; | | | | | |
| | rC2p; rA2p; zc3p | mU; rG; zc3p; zc3p | | | | | |
| | | $ | | | | | |
| CDKN1B_31_S2023 | mC; rA; rG; rC; rG; rC; rA; | mU; rG; rA; rA; rA; rU; | | | | | |
| | rA; rG; mU; rG; rG; rA; | rU2p; rC; mC; rA; rC; | | | | | |
| | rA; rU; rU; rU; mC; rA; | rU; mU; rG; mC; rG; rC; | | | | | |
| | zc3p | mU; rG; zc3p; zc3p | | | | | |
| | | $ | | | | | |
| CDKN1B_31_S2074 | zidB; mC; rA; rG; rC; rG; | mU; rG; rA; rA; rA; rU; | | | | | |
| | rC; rA; rA; rG; rU; rG; | rU2p; rC; mC; rA; rC; | | | | | |
| | rG; rA; rA; rU2p; rU2p; | rU; mU; rG; mC; rG; rC; | | | | | |
| | rU2p; rC2p; rA2p; zc3p | mU; rG; zc3p; zc3p | | | | | |
| | | $ | | | | | |
| ID3_1_S500 | rC; mG; rG; mA; rG; mU; rG; | mU; rU; mC; rA; mC; rA; | | | | 79 48 | 72 |
| | mA; rA; mG; rG; mA; rC; | mG; rU; mC; rC; mU; rU; | | | | at | |
| | mU; rG; mU; rG; mA; rA | mC; rA; mC; rU; mC; | | | | 50 nM) | |
| | | rC; mG | | | | | |
| ID3_2_S500 | rC; mG; rG; mA; rG; mC; rU; | mU; rU; mG; rG; mA; rG; | | | | 55 (5 nM), | 47, |
| | mU; rG; mU; rG; mA; rU; | mA; rU; mC; rA; mC; rA; | | | | 86 at | 93, |
| | mC; rU; mC; rC; mA; rA | mA; rG; mC; rU; mC; | | | | 50 nM) | 86 |
| | | rC; mG | | | | | |
| ID3_5_S500 | rA; mG; rG; mA; rG; mU; rG; | mU; rU; mC; rA; mC; rA; | | | | | 91- |
| | mA; rA; mG; rG; mA; rC; | mG; rU; mC; rC; mU; rU; | | | | | HeLa |
| | mU; rG; mU; rG; mA; rA | mC; rA; mC; rU; mC; | | | | | (17, |
| | | rC; mU | | | | | 14 |
| | | | | | | | RAT1) |
| ID3_9_500 | rA; mG; rC; mU; rU; mA; rG; | mA; rU; mU; rU; mC; rC; | 52, 35 | 52, | | 30, 33- | 35, 31- |
| | mC; rC; mA; rG; mG; rU; | mA; rC; mC; rU; mG; rG; | | 73 | | HeLa | HeLa |
| | mG; rG; mA; rA; mA; rU | mC; rU; mA; rA; mG; | | | | (59- | (10, |
| | | rC; mU | | | | SIRS) | 15 |
| | | | | | | | RAT1) |
| ID3_10_500 | rA; mC; rG; mA; rC; mA; rU; | mU; rA; mG; rC; mA; rG; | 100, 43 | 33 | | 28, 50- | 43- |
| | mG; rA; mA; rC; mC; rA; | mU; rG; mG; rU; mU; rC; | | | | HeLa | HeLa |
| | mC; rU; mG; rC; mU; rA | mA; rU; mG; rU; mC; | | | | (65- | (58%, |
| | | rG; mU | | | | SIRS) | 57%- |
| | | | | | | | SIRS) |
| ID3_11_500 | rG; mA; rC; mA; rU; mG; rA; | mA; rG; mU; rA; mG; rC; | 47, 43, | 56, | | 33, 34- | 43- |
| | mA; rC; mC; rA; mC; rU; | mA; rG; mU; rG; mG; rU; | 59 | 44 | | HeLa | HeLa |
| | mG; rC; mU; rA; mC; rU | mU; rC; mA; rU; mG; | | | | (32- | (6, 6- |
| | | rU; mC | | | | SIRS) | RAT1) |
| ID3_15_S129 | rG; rU; rU; rA; rA; rC; rA; | mU; rA; mU; rU; mA; rU; | | 100 | | | |
| | rU; rU; rU; rU; rG; rC; | mG; rC; mA; rA; mA; rA; | | | | | |
| | rA; rU; rA; rA; LdT; rA | mU; rG; mU; rU; mA; | | | | | |
| | $ | rA; mC$ | | | | | |
| ID3_15_S505 | rG; rU; rU; rA; rA; rC; rA; | rU; mA; rU; mU; rA; mU; | | 100 | | | |
| | rU; rU; rU; rU; rG; rC; | rG; mC; rA; rA; mA; rA; | | | | | |
| | rA; rU; rA; rA; LdT; rA | mU; rG; mU; rU; mA; | | | | | |
| | $ | rA; mC$ | | | | | |
| ID3_16_S129 | rC; rA; rG; rC; rU; rU; rA; | mU; rU; mU; rC; mC; rA; | | 100 | | | 34 |
| | rG; rC; rC; rA; rG; rG; | mC; rC; mU; rG; mG; rC; | | | | | |
| | rU; rG; rG; rA; LdA; rA | mU; rA; mA; rG; mC; | | | | | |
| | $ | rU; mG$ | | | | | |
| ID3_16_S505 | rC; rA; rG; rC; rU; rU; rA; | rU; mU; rU; mC; rC; mA; | | 100 | | | 51 |
| | rG; rC; rC; rA; rG; rG; | rC; mC; rU; rG; mG; rC; | | | | | |
| | rU; rG; rG; rA; LdA; rA | mU; rA; mA; rG; mC; | | | | | |
| | $ | rU; mG$ | | | | | |
| ID3_18_S129 | rA; rG; rG; rA; rA; rG; rG; | mU; rA; mC; rA; mG; rA; | No | | | | |
| | rU; rG; rA; rC; rU; rU; | mA; rA; mG; rU; mC; rA; | data | | | | |
| | rU; rC; rU; rG; LdT; rA | mC; rC; mU; rU; mC; | | | | | |
| | $ | rC; mU$ | | | | | |
| ID3_18_S505 | rA; rG; rG; rA; rA; rG; rG; | rU; mA; rC; mA; rG; mA; | No | | | | |
| | rU; rG; rA; rC; rU; rU; | rA; mA; rG; rU; mC; rA; | data | | | | |
| | rU; rC; rU; rG; LdT; rA | mC; rC; mU; rU; mC; | | | | | |
| | $ | rC; mU$ | | | | | |
| ID3_19_S129 | rU; rG; rA; rA; rC; rU; rC; | mU; rA; mC; rU; mC; rU; | No | | | | |
| | rU; rA; rU; rA; rA; rU; | mA; rU; mU; rA; mU; rA; | data | | | | |
| | rA; rG; rA; rG; LdT; rA | mG; rA; mG; rU; mU; | | | | | |
| | $ | rC; mA$ | | | | | |
| ID3_19_S505 | rU; rG; rA; rA; rC; rU; rC; | rU; mA; rC; mU; rC; mU; | No | | | | |
| | rU; rA; rU; rA; rA; rU; | rA; mU; rU; rA; mU; rA; | data | | | | |
| | rA; rG; rA; rG; LdT; rA | mG; rA; mG; rU; mU; | | | | | |
| | $ | rC; mA$ | | | | | |
| ID3_21_S129 | rC; rA; rG; rG; rA; rA; rG; | mA; rC; mA; rG; mA; rA; | No | | | | |
| | rG; rU; rG; rA; rC; rU; | mA; rG; mU; rC; mA; rC; | data | | | | |
| | rU; rU; rC; rU; LdG; rU | mC; rU; mU; rC; mC; | | | | | |
| | $ | rU; mG$ | | | | | |
| ID3_21_S505 | rC; rA; rG; rG; rA; rA; rG; | rA; mC; rA; mG; rA; mA; | No | | | | |
| | rG; rU; rG; rA; rC; rU; | rA; mG; rU; rC; mA; rC; | data | | | | |
| | rU; rU; rC; rU; LdG; rU | mC; rU; mU; rC; mC; | | | | | |
| | $ | rU; mG$ | | | | | |
| ID3_22_S529 | rC; rU; rC; rU; rA; rU; rA; | mU; rA; mU; rA; mU; rA; | No | | | | |
| | rA; rU; rA; rG; rA; rG; | mC; rU; mC; rU; mA; rU; | data | | | | |
| | rU; rA; rU; rA; LdT; rA | mU; rA; mU; rA; mG; | | | | | |
| | $ | rA; mG$ | | | | | |
| ID3_22_S505 | rC; rU; rC; rU; rA; rU; rA; | rU; mA; rU; mA; rU; mA; | No | | | | |
| | rA; rU; rA; rG; rA; rG; | rC; mU; rC; rU; mA; rU; | data | | | | |
| | rU; rA; rU; rA; LdT; rA | mU; rA; mU; rA; mG; | | | | | |
| | $ | rA; mG$ | | | | | |
| HES1_9_S129 | rG; rA; rU; rG; rA; rC; rA; | mU; rA; mA; rA; mA; rA; | 60, 59 | 74, | | | 32 ± 8 |
| | rU; rU; rU; rC; rG; rU; | mA; rC; mG; rA; mA; rA; | | 28 | | | |
| | rU; rU; rU; rU; LdT; rA | mU; rG; mU; rC; mA; | | | | | |
| | $ | rU; mC$ | | | | | |
| HES1_9_S505 | rG; rA; rU; rG; rA; rC; rA; | rU; mA; rA; mA; rA; mA; | 61, 81 | 67, | | | 32 ± 19 |
| | rU; rU; rU; rC; rG; rU; | rA; mC; rG; rA; mA; rA; | | 25, | | | |
| | rU; rU; rU; rU; LdT; rA | mU; rG; mU; rC; mA; | | | | | |
| | $ | rU; mC$ | | | | | |
| HES1_10_S129 | rG; rU; rA; rU; rU; rA; rA; | mA; rU; mG; rG; mU; rC; | 53, 41 | 46, | | | 26, |
| | rG; rU; rG; rA; rC; rU; | mA; rG; mU; rC; mA; rC; | | 11, | | | 37 |
| | rG; rA; rC; rC; LdA; rU | mU; rU; mA; rA; mU; | | | | | |
| | $ | rA; mC$ | | | | | |
| HES1_10_S505 | rG; rU; rA; rU; rU; rA; rA; | rA; mU; rG; mG; rU; mC; | 72, 68 | 17, | | | 19, |
| | rG; rU; rG; rA; rC; rU; | rA; mG; rU; rC; mA; rC; | | 13 | | | 46 |
| | rG; rA; rC; rC; LdA; rU | mU; rU; mA; rA; mU; | | | | | |
| | $ | rA; mC$ | | | | | |
| HES1_11_S129 | rG; rA; rA; rA; rA; rC; rA; | mA; rU; mC; rC; mA; rA; | 76, 73 | 48, | | | 79, |
| | rC; rU; rG; rA; rU; rU; | mA; rA; mU; rC; mA; rG; | | 65 | | | 79 |
| | rU; rU; rG; rG; LdA; rU | mU; rG; mU; rU; mU; | | | | | |
| | $ | rU; mC$ | | | | | |
| HES1_11_S505 | rG; rA; rA; rA; rA; rC; rA; | rA; mU; rC; mC; rA; mA; | 95, | 23, | | | 89, |
| | rC; rU; rG; rA; rU; rU; | rA; mA; rU; rC; mA; rG; | 101 | 29 | | | 124 |
| | rU; rU; rG; rG; LdA; rU | mU; rG; mU; rU; mU; | | | | | |
| | $ | rU; mC$ | | | | | |
| HES1_14_S709 | rC; rA; rG; rC; rG; rA; rG; | rU; rU; rC; rG; rU; rU; | | | | | |
| | rU; rG; rC; rA; rU; rG; | rC; rA; rU; rG; rC; rA; | | | | | |
| | rA; rA; rC; rG; rA; rA; | rC; rU; rC; rG; rC; | | | | | |
| | zdT; zdT$ | rU; rG; zdT; zdT$ | | | | | |
| HES1_14_S1964 | zidB; mC; rA; rG; rC; rG; | mU; rU; mC; rG; rU; rU; | | | | | |
| | rA; rG; rU; rG; rC; rA; | rC2p; rA; mU; rG; mC; | | | | | |
| | rU; rG; rA; rA2p; rC2p; | rA; rC; mU; mC; rG; rC; | | | | | |
| | rG2p; rA2p; rA2p; zc3p | mU; rG; zc3p; zc3p | | | | | |
| | | $ | | | | | |
| HES1_14_S1965 | zidB; mC; rA; rG; mC; rG; | mU; rU; mC; rG; rU; rU; | | | | | |
| | rA; rG; mU; rG; mC; rA; | rC2p; rA; mU; rG; mC; | | | | | |
| | mU; rG; rA; rA; mC; rG; rA; | rA; rC; mU; mC; rG; rC; | | | | | |
| | rA; zc3p | mU; rG; zc3p; zc3p | | | | | |
| | | $ | | | | | |
| HES1_14_S2057 | zidB; mC; rA; rG; rC; rG; | mU; rU; mC; rG; rU; rU; | | | | | |
| | rA; rG; rU; rG; rC; rA; | rC; rA; mU; rG; mC; rA; | | | | | |
| | rU; rG; rA; rA2p; rC2p; | rC; rU; mC; rG; rC; | | | | | |
| | rG2p; rA2p; rA2p; zc3p | mU; rG; zc3p; zc3p$ | | | | | |
| HES1_22_S709 | rC; rA; rG; rU; rG; rU; rC; | rU; rG; rG; rU; rG; rU; | | | | | |
| | rA; rA; rC; rA; rC; rG; | rC; rG; rU; rG; rU; rU; | | | | | |
| | rA; rC; rA; rC; rC; rA; | rG; rA; rC; rA; rC; | | | | | |
| | zdT; zdT$ | rU; rG; zdT; zdT$ | | | | | |
| HES1_24_S709 | rG; rG; rC; rG; rG; rA; rC; | rU; rC; rU; rC; rC; rA; | | | | | |
| | rU; rC; rC; rA; rU; rG; | rC; rA; rU; rG; rG; rA; | | | | | |
| | rU; rG; rG; rA; rG; rA; | rG; rU; rC; rC; rG; | | | | | |
| | zdT; zdT$ | rC; rC; zdT; zdT$ | | | | | |
| HES1_26_S709 | rC; rA; rG; rU; rG; rA; rA; | rU; rU; rC; rC; rG; rG; | | | | | |
| | rG; rC; rA; rC; rC; rU; | rA; rG; rG; rU; rG; rC; | | | | | |
| | rC; rC; rG; rG; rA; rA; | rU; rU; rC; rA; rC; | | | | | |
| | zdT; zdT$ | rU; rG; zdT; zdT$ | | | | | |
| HES1_27_S709 | rC; rA; rU; rG; rG; rA; rG; | rU; rC; rU; rU; rC; rG; | | | | | |
| | rA; rA; rA; rA; rG; rA; | rU; rC; rU; rU; rU; rU; | | | | | |
| | rC; rG; rA; rA; rG; rA; | rC; rU; rC; rC; rA; | | | | | |
| | zdT; zdT$ | rU; rG; zdT; zdT$ | | | | | |
| HES1_28_ S709 | rC; rG; rG; rA; rU; rA; rA; | rU; rC; rU; rG; rU; rC; | | | | | |
| | rA; rC; rC; rA; rA; rA; | rU; rU; rU; rG; rG; rU; | | | | | |
| | rG; rA; rC; rA; rG; rA; | rU; rU; rA; rU; rC; | | | | | |
| | zdT; zdT$ | rC; rG; zdT; zdT$ | | | | | |
| HES1_30_S2037 | zidB; mC; rA; rG; rC; mU; | rA; rU; mC; mU; rC; rC | | | | | |
| | rG; rA; mU; rA; mU; rA; | 2p; rA; rU; mU; rA; mU; | | | | | |
| | rA; mU; rG; rG; rA; rG; rA; | rA; rU; rC; mA; rG; rC; | | | | | |
| | rU; zc3p | mU; rG; zc3p; zc3p | | | | | |
| | | $ | | | | | |
| HES1_30_S2038 | zidB; mC; rA; rG; rC; mU; | rA; rU; mC; rU; mC; rC | | | | | |
| | rG; rA; mU; rA; mU; rA; | 2p; rA; rU; mU; rA; mU; | | | | | |
| | rA; mU; rG; rG; rA; rG; rA; | rA; rU; rC; mA; rG; rC; | | | | | |
| | rU; zc3p | mU; rG; zc3p; zc3p | | | | | |
| | | $ | | | | | |
| HES1_30_S2039 | zidB; mC; rA; rG; rC; mU; | rA; mU; mC; mU; rC; rC | | | | | |
| | rG; rA; mU; rA; mU; rA; | 2p; rA; rU; mU; rA; mU; | | | | | |
| | rA; mU; rG; rG; rA; rG; rA; | rA; rU; mC; rA; rG; mC; | | | | | |
| | rU; zc3p | mU; rG; zc3p; zc3p | | | | | |
| | | $ | | | | | |
| HES1_30_S2040 | zidB; mC; rA; rG; rC; mU; | rA; rU; mC; mU; rC; rC | | | | | |
| | rG; rA; mU; rA; mU; rA; | 2p; rA; rU; mU; rA; mU; | | | | | |
| | rA; mU; rG; rG2p; rA2p; | rA; rU; rC; mA; rG; rC; | | | | | |
| | rG2p; rA2p; rU2p; zc3p | mU; rG; zc3p; zc3p | | | | | |
| | | $ | | | | | |
| HES1_30_S2041 | zidB; mC; rA; rG; rC; mU; | rA; rU; mC; rU; mC; rC | | | | | |
| | rG; rA; mU; rA; mU; rA; | 2p; rA; rU; mU; rA; mU; | | | | | |
| | rA; mU; rG; rG2p; rA2p; | rA; rU; rC; mA; rG; rC; | | | | | |
| | rG2p; rA2p; rU2p; zc3p | mU; rG; zc3p; zc3p | | | | | |
| | | $ | | | | | |
| HES1_30_S2042 | zidB; mC; rA; rG; rC; mU; | rA; mU; mC; mU; rC; rC | | | | | |
| | rG; rA; mU; rA; mU; rA; | 2p; rA; rU; mU; rA; mU; | | | | | |
| | rA; mU; rG; rG2p; rA2p; | rA; rU; mC; rA; rG; mC; | | | | | |
| | rG2p; rA2p; rU2p; zc3p | mU; rG; zc3p; zc3p | | | | | |
| | | $ | | | | | |
| HES1_33_S709 | rA; rG; rU; rG; rC; rA; rU; | rU; rU; rC; rA; rC; rC; | | | | | |
| | rG; rA; rA; rC; rG; rA; | rU; rC; rG; rU; rU; rC; | | | | | |
| | rG; rG; rU; rG; rA; rA; | rA; rU; rG; rC; rA; | | | | | |
| | zdT; zdT$ | rC; rU; zdT; zdT$ | | | | | |
| HES1_34_S2056 | zc6Np; rC; rG; rG; rA; rC; | rU; rC; rU; rG; rU; rC; | | | | | |
| | rA; rA; rA; rC; rC; rA; | rU; rU; rU; rG; rG; rU; | | | | | |
| | rA; rA; rG; rA; rC; rA; | rU; rU; rG; rU; rC; | | | | | |
| | rG; rA; zdT; zdT$ | rC; rG; zdT; zdT$ | | | | | |
| HES1_36_S2086 | zidB; rC; rA; rG; rC; rG; | rA; rU; mC; rG; rU; rU; | | | | | |
| | rA; rG; rU; rG; rC; rA; | rC; rA; mU; rG; mC; rA; | | | | | |
| | rU; rG; rA; rA2p; rC2p; | rC; rU; mC; rG; rC; | | | | | |
| | rG2p; rA2p; rU2p; zc3p | rU; rG; zc3p; zc3p | | | | | |
| HES5_8_S73 | rG; mG; rG; mU; rU; mC; rU; | mU; rA; mC; rA; mA; rA; | 72, 71 | | 59 | 96 | 80% |
| | mA; rU; mG; rA; mU; rA; | mU; rA; mU; rC; mA; rU; | | | | | (5 nM) |
| | mU; rU; mU; rG; mU; rA$ | mA; rG; mA; rA; mC; | | | | | |
| | | rC; mC$ | | | | | |
| HES5_8_S500 | rG; mG; rG; mU; rU; mC; rU; | mU; rA; mC; rA; mA; rA; | 61, | | 40 | 8, 8 | 9, 7 |
| | mA; rU; mG; rA; mU; rA; | mU; rA; mU; rC; mA; rU; | 65 | | | | |
| | mU; rU; mU; rG; mU; rA | mA; rG; mA; rA; mC; | | | | | |
| | | rC; mC | | | | | |
| HES5_10_S500 | rC; mU; rG; mU; rA; mG; rA; | mU; rG; mA; rA; mG; rA; | 18, | | | | 4, 12 |
| | mG; rG; mA; rC; mU; rU; | mA; rA; mG; rU; mC; rC; | 31, 55 | | | | |
| | mU; rC; mU; rU; mC; rA | mU; rC; mU; rA; mC; | | | | | |
| | | rA; mG | | | | | |
| HES5_11_S500 | rC; mC; rG; mU; rG; mU; rU; | mU; rU; mG; rU; mC; rC; | | | | 6, 10 | |
| | mG; rU; mU; rU; mG; rA; | mU; rC; mA; rA; mA; rC; | | | | | |
| | mG; rG; mA; rC; mA; rA | mA; rA; mC; rA; mC; | | | | | |
| | | rG; mG | | | | | |
| HES5_12_S500 | rG; mC; rA; mC; rU; mU; rU; | mU; rU; mC; rA; mC; rA; | | | | 36, 69 | |
| | mG; rC; mC; rU; mU; rU; | mA; rA; mA; rG; mG; rC; | | | | | |
| | mU; rG; mU; rG; mA; rA | mA; rA; mA; rG; mU; | | | | | |
| | | rG; mC | | | | | |
| HES5_13_S500 | rA; mG; rG; mU; rG; mU; rA; | mU; rU; mC; rC; mU; rA; | | | | 28 | |
| | mU; rC; mC; rU; mC; rA; | mU; rG; mA; rG; mG; rA; | | | | | |
| | mU; rA; mG; rG; mA; rA | mU; rA; mC; rA; mC; | | | | | |
| | | rC; mU | | | | | |
| HES5_14_S500 | rA; mG; rG; mU; rG; mU; rA; | mU; rU; mC; rC; mU; rA; | | | | | 19, |
| | mU; rC; mU; rU; mC; rA; | mU; rG; mA; rA; mG; rA; | | | | | 34 |
| | mU; rA; mG; rG; mA; rA | mU; rA; mC; rA; mC; | | | | | |
| | | rC; mU | | | | | |
| HES5_8_S211 | rG; rG; rG; rU; rU; rC; rU; | mU; rA; mC; rA; mA; rA; | | | | 45 | 52 |
| | rA; rU; rG; rA; rU; rA; | mU; rA; mU; rC; mA; rU; | | | | | |
| | rU; dT; rU; rG; LdT; rA | mA; rG; mA; rA; mC; | | | | | |
| | $ | rC; mC$ | | | | | |
| HES5_8_S215 | rG; rG; rG; rU; rU; rC; rU; | mU; rA; mC; rA; mA; rA; | | | | 37 | 58 |
| | rA; rU; rG; rA; rU; rA; | mU; rA; mU; rC; mA; rU; | | | | | |
| | rU; dT; LdT; rG; LdT; r | mA; rG; mA; rA; mC; | | | | | |
| | A$ | rC; mC$ | | | | | |
| HES5_8_S219 | iB; rG; rG; rG; rU; rU; rC; | mU; rA; mC; rA; mA; rA; | | | | 14 | 61 |
| | rU; rA; rU; rG; rA; rU; | mU; rA; mU; rC; mA; rU; | | | | | |
| | rA; rU; dT; rU; rG; LdT; | mA; rG; mA; rA; mC; | | | | | |
| | rA$ | rC; mC$ | | | | | |
| HES5_8_S220_M1 | LdT; rG; rG; rG; rU; rU; | mU; rA; mC; rA; mA; rA; | | | | 30 | 76 |
| | rC; rU; rA; rU; rG; rA; rU; | mU; rA; mU; rC; mA; rU; | | | | | |
| | rA; rU; dT; LdT; rG; LdT; | mA; rG; mA; rA; mC; | | | | | |
| | rA$ | rC; mC$ | | | | | |
| HES5_8_S221 | c6Np; rG; rG; rG; rU; rU; | mU; rA; mC; rA; mA; rA; | | | | 23 | 63 |
| | rC; rU; rA; rU; rG; rA; | mU; rA; mU; rC; mA; rU; | | | | | |
| | rU; rA; rU; dT; rU; rG; LdT; | mA; rG; mA; rA; mC; | | | | | |
| | rA$ | rC; mC$ | | | | | |
| HES5_8_S222 | c6Np; rG; rG; rG; rU; rU; | mU; rA; mC; rA; mA; rA; | | | | 21 | 81 |
| | rC; rU; rA; rU; rG; rA; | mU; rA; mU; rC; mA; rU; | | | | | |
| | rU; rA; rU; dT; LdT; rG; | mA; rG; mA; rA; mC; | | | | | |
| | LdT; rA$ | rC; mC$ | | | | | |
| HES5_8_S226_M1 | LdT; rG; rG; rG; rU; rU; | rU; mA; rC; mA; rA; mA; | | | | 82 | 99 |
| | rC; rU; rA; rU; rG; rA; rU; | rU; mA; rU; rC; mA; rU; | | | | | |
| | rA; rU; dT; LdT; rG; LdT; | mA; rG; mA; rA; mC; | | | | | |
| | rA$ | rC; mC$ | | | | | |
| HES5_8_S227 | c6Np; rG; rG; rG; rU; rU; | rU; mA; rC; mA; rA; mA; | | | | 24 | 95 |
| | rC; rU; rA; rU; rG; rA; | rU; mA; rU; rC; mA; rU; | | | | | |
| | rU; rA; rU; dT; rU; rG; LdT; | mA; rG; mA; rA; mC; | | | | | |
| | rA$ | rC; mC$ | | | | | |
| HES5_8_S228 | c6Np; rG; rG; rG; rU; rU; | rU; mA; rC; mA; rA; mA; | | | | 42 | 83 |
| | rC; rU; rA; rU; rG; rA; | rU; mA; rU; rC; mA; rU; | | | | | |
| | rU; rA; rU; dT; LdT; rG; | mA; rG; mA; rA; mC; | | | | | |
| | LdT; rA$ | rC; mC$ | | | | | |
| HES5_8_S234 | rG; rG; rG; rU; rU; rC; rU; | rU; mA; rC; mA; rA; mA; | | | | 139 | 87 |
| | rA; rU; rG; rA; rU; rA; | rU; mA; rU; rC; mA; rU; | | | | | |
| | rU; dT; LdT; rG; LdT; | mA; rG; mA; rA; mC; | | | | | |
| | rA$ | rC; mC$ | | | | | |
| HES5_8_S387_M1 | LdT; rG; rG; rG; rU; rU; | mU; rA; mC; rA; mA; rA; | | | | 23 | 56 |
| | rC; rU; rA; rU; rG; rA; rU; | mU; rA; mU; rC; mA; rU; | | | | | |
| | rA; rU; dT; rU; rG; LdT; | mA; rG; mA; rA; mC; | | | | | |
| | rA$ | rC; mC$ | | | | | |
| HESS_8_S225 | iB; rG; rG; rG; rU; rU; rC; | rU; mA; rC; mA; rA; mA; | | | | 38 | 46 |
| | rU; rA; rU; rG; rA; rU; | rU; mA; rU; rC; mA; rU; | | | | | (5 nM) |
| | rA; rU; dT; rU; rG; LdT; | mA; rG; mA; rA; mC; | | | | | |
| | rA$ | rC; mC$ | | | | | |
| HES5_8_S526 | iB; rG; rG; rG; rU; rU; rC; | rU; mA; rC; mA; rA; mA; | | | | 77 | 69 |
| | rU; rA; rU; rG; rA; rU; | rU; mA; rU; rC; mA; rU; | | | | | |
| | rA; rU; dT; LdT; rG; LdT; | mA; rG; mA; rA; mC; | | | | | |
| | rA$ | rC; mC$ | | | | | |
| HES5_8_S527_M1 | LdT; rG; rG; rG; rU; rU; | rU; mA; rC; mA; rA; mA; | | | | 28 | 70 |
| | rC; rU; rA; rU; rG; rA; rU; | rU; mA; rU; rC; mA; rU; | | | | | |
| | rA; rU; dT; rU; rG; LdT; | mA; rG; mA; rA; mC; | | | | | |
| | rA$ | rC; mC$ | | | | | |
| HES5_8_S233 | rG; rG; rG; rU; rU; rC; rU; | rU; mA; rC; mA; rA; mA; | | | | 18 | 89 |
| | rA; rU; rG; rA; rU; rA; | rU; mA; rU; rC; mA; rU; | | | | | |
| | rU; dT; rU; rG; LdT; rA | mA; rG; mA; rA; mC; | | | | | |
| | $ | rC; mC$ | | | | | |
| HES5_8_S459 | iB; rG; rG; rG; rU; rU; rC; | mU; rA; mC; rA; mA; rA; | | | | 17 | 54 |
| | rU; rA; rU; rG; rA; rU; | mU; rA; mU; rC; mA; rU; | | | | | |
| | rA; rU; dT; LdT; rG; LdT; | mA; rG; mA; rA; mC; | | | | | |
| | rA$ | rC; mC$ | | | | | |
| NOTCH1_1_S709 | rC; rC; rU; rU; rC; rU; rA; | rU; rG; rA; rC; rA; rC; | | | | | |
| | rC; rU; rG; rC; rG; rA; | rU; rC; rG; rC; rA; rG; | | | | | |
| | rG; rU; rG; rU; rC; rA; | rU; rA; rG; rA; rA; | | | | | |
| | zdT; zdT$ | rG; rG; zdT; zdT$ | | | | | |
| NOTCH1_2_S709 | rG; rC; rU; rA; rC; rA; rA; | rU; rC; rA; rC; rA; rC; | | | | | |
| | rC; rU; rG; rC; rG; rU; | rA; rC; rG; rC; rA; rG; | | | | | |
| | rG; rU; rG; rU; rG; rA; | rU; rU; rG; rU; rA; | | | | | |
| | zdT; zdT$ | rG; rC; zdT; zdT$ | | | | | |
| NOTCH1_3_S709 | rU; rC; rC; rU; rU; rC; rU; | rU; rA; rC; rA; rC; rU; | | | | | |
| | rA; rC; rU; rG; rC; rG; | rC; rG; rC; rA; rG; rU; | | | | | |
| | rA; rG; rU; rG; rU; rA; | rA; rG; rA; rA; rG; | | | | | |
| | zdT; zdT$ | rG; rA; zdT; zdT$ | | | | | |
| NOTCH1_4_S709 | rC; rU; rC; rC; rU; rU; rC; | rU; rC; rA; rC; rU; rC; | | | | | |
| | rU; rA; rC; rU; rG; rC; | rG; rC; rA; rG; rU; rA; | | | | | |
| | rG; rA; rG; rU; rG; rA; | rG; rA; rA; rG; rG; | | | | | |
| | zdT; zdT$ | rA; rG; zdT; zdT$ | | | | | |
| NOTCH1_5_S709 | rC; rA; rG; rC; rG; rC; rA; | rU; rA; rU; rG; rU; rU; | | | | | |
| | rG; rA; rU; rG; rC; rC; | rG; rG; rC; rA; rU; rC; | | | | | |
| | rA; rA; rC; rA; rU; rA; | rU; rG; rC; rG; rC; | | | | | |
| | zdT; zdT$ | rU; rG; zdT; zdT$ | | | | | |
| NOTCH1_6_S709 | rA; rC; rA; rA; rC; rU; rG; | rU; rU; rG; rA; rC; rA; | | | | | |
| | rC; rG; rU; rG; rU; rG; | rC; rA; rC; rA; rC; rG; | | | | | |
| | rU; rG; rU; rC; rA; rA; | rC; rA; rG; rU; rU; | | | | | |
| | zdT; zdT$ | rG; rU; zdT; zdT$ | | | | | |
| NOTCH1_7_S709 | rG; rG; rG; rA; rC; rA; rA; | rU; rG; rA; rU; rG; rU; | | | | | |
| | rA; rC; rU; rG; rU; rG; | rC; rA; rC; rA; rG; rU; | | | | | |
| | rA; rC; rA; rU; rC; rA; | rU; rU; rG; rU; rC; | | | | | |
| | zdT; zdT$ | rC; rC; zdT; zdT$ | | | | | |
| ID3_38_S1952 | zidB; mC; rG; rA; rC; rA; | p; yrA; mU; rA; rG; mC; | 55 | | | | |
| | rU; rG; rA; rA; rC; rC; | rA; rG; mU; rG; rG; rU; | | | | | |
| | rA; rC; rU; rG2p; rG2p; | rU; mC; rA; mU; rG; | | | | | |
| | rU2p; rA2p; yrU2p; zc3p | rU; mC; rG; zc3p; zc3p | | | | | |
| ID3_32_S1953 | zidB; mU; rG; mU; rG; rC; | p; mU; rG; rU; rU; rC; | 23 | | | | |
| | rU; rG; rC; rC; rU; rG; | mC; rG; rA; mC; rA; rG; | | | | | |
| | rU; rC; rG; rG2p; rA2p; | rG; mC; rA; rG; mC; rA; | | | | | |
| | rA2p; rC2p; rA2p; zc3p | mC; rA; zc3p; zc3p | | | | | |
| HEY2_1_S1929 | zidB; rG; rG; rG; rA; rG; | mU; rG; mU; rA; rA; rU; | | | | | |
| | mC; rG; rA; rG; rA; rA; | rU2p; rG; mU; rU; mC; | | | | | |
| | mC; rA; rA; rU; mU; rA; mC; | rU; mC; rG; mC; rU; m | | | | | |
| | rA; zc3p | C; rC; mC; zc3p; zc3p | | | | | |
| HEY2_2_S1970 | zidB; rG; rG; rG; mU; rA; | mU; rU; mC; rA; rA; rA; | | | | | |
| | rA; rA; rG; rG; rC; mU; | rG2p; mU; rA; rG; mC; | | | | | |
| | rA; mC; rU; rU; mU; rG; rA; | rC; mU; rU; mU; rA; mC; | | | | | |
| | rA; zc3p | rC; mC; zc3p; zc3p | | | | | |
| NOTCH1_2_S2082 | zidB; rG; rC; rU; rA; rC; | mU; rC; mA; rC; mA; rC; | | | | | |
| | rA; rA; rC; rU; rG; rC; | rA2p; rC; mG; rC; mA; | | | | | |
| | rG; rU; rG; mU; rG; mU; rG; | rG; rU; rU; rG; rU; mA; | | | | | |
| | rA; zc3p | rG; rC; zc3p; zc3p | | | | | |
| NOTCH1_2_S2083 | zidB; rG; rC; rU; rA; rC; | mU; rC; rA; rC; rA; rC; | | | | | |
| | rA; rA; rC; rU; rG; rC; | rA2p; rC; rG; rC; rA; | | | | | |
| | rG; rU; rG; mU; rG; mU; rG; | rG; rU; rU; rG; rU; mA; | | | | | |
| | rA; zc3p | rG; rC; zc3p; zc3p | | | | | |
| NOTCH1_2_S2084 | zidB; rG; rC; rU; rA; rC; | mU; rC; mA; rC; mA; rC; | | | | | |
| | rA; rA; rC; rU; rG; rC; | rA2p; rC; mG; rC; mA | | | | | |
| | rG; rU; rG; rU2p; rG2p; | ; rG; rU; rU; rG; rU; mA; | | | | | |
| | rU2p; rG2p; rA2p; zc3p | rG; rC; zc3p; zc3p | | | | | |
| NOTCH1_2_S2085 | zidB; rG; rC; rU; rA; rC; | mU; rC; rA; rC; rA; rC; | | | | | |
| | rA; rA; rC; rU; rG; rC; | rA2p; rC; rG; rC; rA; | | | | | |
| | rG; rU; rG; rU2p; rG2p; | rG; rU; rU; rG; rU; mA; | | | | | |
| | rU2p; rG2p; rA2p; zc3p | rG; rC; zc3p; zc3p | | | | | |

Table C hereinbelow provides a legend of the modified ribonucleotides/unconventional moieties utilized in preparing the dsRNA molecules disclosed herein.

TABLE C
Legend

| Code | Modification |
|---|---|
| Nuc | |
| 5medG | 5-methyl-deoxyriboguanosine-3′-phosphate |
| c6Np | Amino modifier C6 (Glen Research 10-1906-xx) |
| dA | deoxyriboadenosine-3′-phosphate |
| dB | abasic deoxyribose-3′-phosphate |
| dC | deoxyribocytidine-3′-phosphate |
| dG | deoxyriboguanosine-3′-phosphate |
| dT | thymidine-3′-phosphate |
| dT$ | thymidine (no phosphate) |
| enaA$ | ethylene-bridged nucleic acid adenosine (no phosphate) |
| enaC | ethylene-bridged nucleic acid cytidine 3′ phosphate |
| enaG | ethylene-bridged nucleic acid guanosine 3′ phosphate |
| enaT | ethylene-bridged nucleic acid thymidine 3′ phosphate |
| iB | inverted deoxyabasic |
| LdA | L-deoxyriboadenosine-3′-phosphate (mirror image dA) |
| LdA$ | L-deoxyriboadenosine (no phosphate) (mirror image dA) |
| LdC | L-deoxyribocytidine-3′-phosphate (mirror image dC) |
| LdC$ | L-deoxyribocytidine (no phosphate) (mirror image dC) |
| LdG | L-deoxyriboguanosine-3′-phosphate (mirror image dG) |
| LdT | L-deoxyribothymidine-3′-phosphate (mirror image dT) |
| LdT$ | L-deoxyribothymidine (no phosphate) (mirror image dT) |
| mA | 2′-O-methyladenosine-3′-phosphate |
| mA$ | 2′-O-methyladenosine (no phosphate) |
| mC | 2′-O-methylcytidine-3′-phosphate |
| mC$ | 2′-O-methylcytidine (no 3′-phosphate) |
| mG | 2′-O-methylguanosine-3′-phosphate |
| mG$ | 2′-O-methylguanosine (no phosphate) |
| mU | 2′-O-methyluridine-3′-phosphate |
| mU$ | 2′-O-methyluridine (no phosphate) |
| rA | riboadenosine-3′-phosphate |
| rA$ | riboadenosine (no phosphate) |
| rA2p | riboadenosine-2′-phosphate |
| rC | ribocytidine-3′-phosphate |
| rC$ | ribocytidine (no phosphate) |
| rC2p | ribocytidine-2′-phosphate |
| rG | riboguanosine-3′-phosphate |
| rG2p | riboguanosine-2′-phosphate |
| rU | ribouridine-3′-phosphate |
| rU$ | ribouridine (no phosphate) |
| rU2p | ribouridine-2′-phosphate |
| P | 5′-phosphate |
| z | Prefix for Capping moiety |
| zc3p | C3Pi covalently attached |
| zc3p$ | C3OH covalently attached |
| $ | No terminal phosphate |

TABLE D
Activity (% residual mRNA) in HeLa cells using the psiCHECK
system on the AS-CM (Antisense complete match) sequence

| | siRNA dose | Exp 1 | Exp 2 | Exp 3 |
|---|---|---|---|---|
| Hes1 14 S709 | 100 nM | | | 5 |
| | 20 nM | 6 | 13 | |
| | 33.3 nM | | | 6 |
| | 11.1 nM | | | 5 |
| | 5 nM | 8 | 16 | |
| | 3.7 nM | | | 6 |
| | 1.25 nM | 13 | 12 | 7 |
| | 0.412 nM | | | 12 |
| | 0.3125 nM | 18 | 17 | |
| | 0.137 nM | | | 15 |
| | 0.078 nM | 24 | 19 | |
| | 0.046 nM | | | 20 |
| | 0.019 nM | 33 | 28 | |
| | 0.015 nM | | | 24 |
| | 0.0049 nM | 37 | 36 | 25 |
| | 0.0012 nM | 41 | 65 | |
| | 0.00031 nM | 44 | 47 | |
| | 0.000076 nM | 48 | 42 | |
| Hes1 14 S2057 | 20 nM | 10 | | |
| | 5 nM | 15 | | |
| | 1.25 nM | 20 | | |
| | 0.3125 nM | 30 | | |
| | 0.078 nM | 39 | | |
| | 0.019 nM | 47 | | |
| | 0.0049 nM | 51 | | |
| | 0.0012 nM | 52 | | |
| | 0.00031 nM | 54 | | |
| | 0.000076 nM | 57 | | |
| HES1 14 S1964 | 100 nM | | | 7 |
| | 33.3 nM | | | 8 |
| | 11.1 nM | | | 10 |
| | 3.7 nM | | | 14 |
| | 1.23 nM | | | 24 |
| | 0.412 nM | | | 38 |
| | 0.137 nM | | | 45 |
| | 0.046 nM | | | 57 |
| | 0.015 nM | | | 80 |
| | 0.005 nM | | | 72 |
| HES1 14 S2057 | 100 nM | | | 3 |
| | 33.3 nM | | | 4 |
| | 11.1 nM | | | 4 |
| | 3.7 nM | | | 5 |
| | 1.23 nM | | | 7 |
| | 0.412 nM | | | 12 |
| | 0.137 nM | | | 21 |
| | 0.046 nM | | | 36 |
| | 0.015 nM | | | 49 |
| | 0.005 nM | | | 60 |
| Hes1 36 S2086 | 100 nM | | | 3 |
| | 33.3 nM | | 11 | |
| | 20 nM | 9 | 13 | 4 |
| | 11.1 nM | | | 4 |
| | 5 nM | 11 | 14 | |
| | 3.7 nM | | | 4 |
| | 1.25 nM | 14 | 14 | |
| | 0.412 nM | | | 7 |
| | 0.3125 nM | 19 | 21 | |
| | 0.137 nM | | | 9 |
| | 0.078 nM | 23 | 30 | |
| | 0.046 nM | | | 14 |
| | 0.019 nM | 28 | 97 | 24 |
| | 0.0049 nM | 32 | 157 | 37 |
| | 0.0012 nM | 28 | 106 | |
| | 0.00031 nM | 39 | 158 | |
| | 0.000076 nM | 43 | | |

TABLE E
Activity (% residual mRNA) in rat REF52 cells by qPCR
Results are residual (% of Ctrl) rat HES1 gene

| | siRNA dose | Exp 1 | Exp 2 |
|---|---|---|---|
| HES1 14 S709 | 100 nM | 60 | |
| | 50 nM | 49 | 51 |
| | 25 nM | 62 | 62 |
| HES1 14 S2057 | 100 nM | 74 | |
| | 50 nM | 54 | |
| | 25 nM | 78 | |
| HES1 36 S2086 | 100 nM | 70 | |
| | 50 nM | 54 | |
| | 25 nM | 53 | |
| HES1 14 S1964 50 nM | 50 nM | | 88 |
| | 25 nM | | 90 |
| HES1 14 S1965 50 nM | 50 nM | | 101 |
| | 25 nM | | 196 |

TABLE F
Activity (% residual mRNA) in rat Rat-1 cells exogenously
expressing guinea pig HES1 gene by qPCR

| | siRNA dose | Exp 1 | Exp 2 | Exp 3 |
|---|---|---|---|---|
| HES1 14 S709 | 80 nM | | 19 | |
| | 50 nM | 23 | | |
| | 40 nM | | 23 | 23 |
| | 25 nM | 24 | | |
| | 20 nM | | 25 | 22 |
| | 5 nM | 40 | | 23 |
| | 1 nM | | | 48 |
| HES1 14 S2057 | 80 nM | | 30 | |
| | 50 nM | 21 | | |
| | 40 nM | | 26 | |
| | 25 nM | 35 | | |
| | 20 nM | | | |
| | 5 nM | 49 | 40 | |
| HES1 36 S2086 | 50 nM | 20 | | |
| | 25 nM | 35 | | |
| | 5 nM | 32 | | |
| Hes1 35 S2047 | 80 nM | | 29 | |
| | 40 nM | | 31 | 19 |
| | 20 nM | | 42 | 46 |
| | 5 nM | | | 32 |
| | 1 nM | | | 89 |

TABLE G
Activity (% residual mRNA): siCDKN1B_4
Antisense complete match psiCHECK

| | SiRNA | |
|---|---|---|
| | dose | KD |
| CDKN1B 4 S2018 | 100 nM | 8 |
| | 33.3 nM | 12 |
| | 11.1 nM | 9 |
| | 3.7 nM | 9 |
| | 1.23 nM | 11 |
| | 0.41 nM | 13 |
| | 0.137 nM | 30 |
| | 0.045 nM | 49 |
| | 0.015 nM | 61 |
| | 0.005 nM | 69 |
| CDKN1B 4 S2075 | 100 nM | 8 |
| | 33.3 nM | 7 |
| | 11.1 nM | 11 |
| | 3.7 nM | 9 |
| | 1.23 nM | 16 |
| | 0.41 nM | 16 |
| | 0.137 nM | 31 |
| | 0.045 nM | 50 |
| | 0.015 nM | 59 |
| | 0.005 nM | 60 |
| CDKN1B 4 S2076 | 100 nM | 6 |
| | 33.3 nM | 7 |
| | 11.1 nM | 5 |
| | 3.7 nM | 10 |
| | 1.23 nM | 20 |
| | 0.41 nM | 28 |
| | 0.137 nM | 43 |
| | 0.045 nM | 76 |
| | 0.015 nM | 72 |
| | 0.005 nM | 71 |

TABLE H
siCDKN1B_4 Sense complete match psiCHECK to show
off-target knock-down potential of the sense strands.

| | SiRNA | |
|---|---|---|
| | dose | KD |
| CDKN1B 4 S2018 | 100 nM | 13 |
| | 33.3 nM | 9 |
| | 11.1 nM | 14 |
| | 3.7 nM | 17 |
| | 1.23 nM | 22 |
| | 0.41 nM | 36 |
| | 0.137 nM | 66 |
| | 0.045 nM | 73 |
| | 0.015 nM | 83 |
| | 0.005 nM | 66 |
| CDKN1B 4 S2075 | 100 nM | 25 |
| | 33.3 nM | 23 |
| | 11.1 nM | 31 |
| | 3.7 nM | 39 |
| | 1.23 nM | 49 |
| | 0.41 nM | 53 |
| | 0.137 nM | 62 |
| | 0.045 nM | 78 |
| | 0.015 nM | 77 |
| | 0.005 nM | 77 |
| CDKN1B_4_S2076 | 100 nM | 44 |
| | 33.3 nM | 69 |
| | 11.1 nM | 71 |
| | 3.7 nM | 64 |
| | 1.23 nM | 69 |
| | 0.41 nM | 76 |
| | 0.137 nM | 71 |
| | 0.045 nM | 83 |
| | 0.015 nM | 89 |
| | 0.005 nM | 78 |

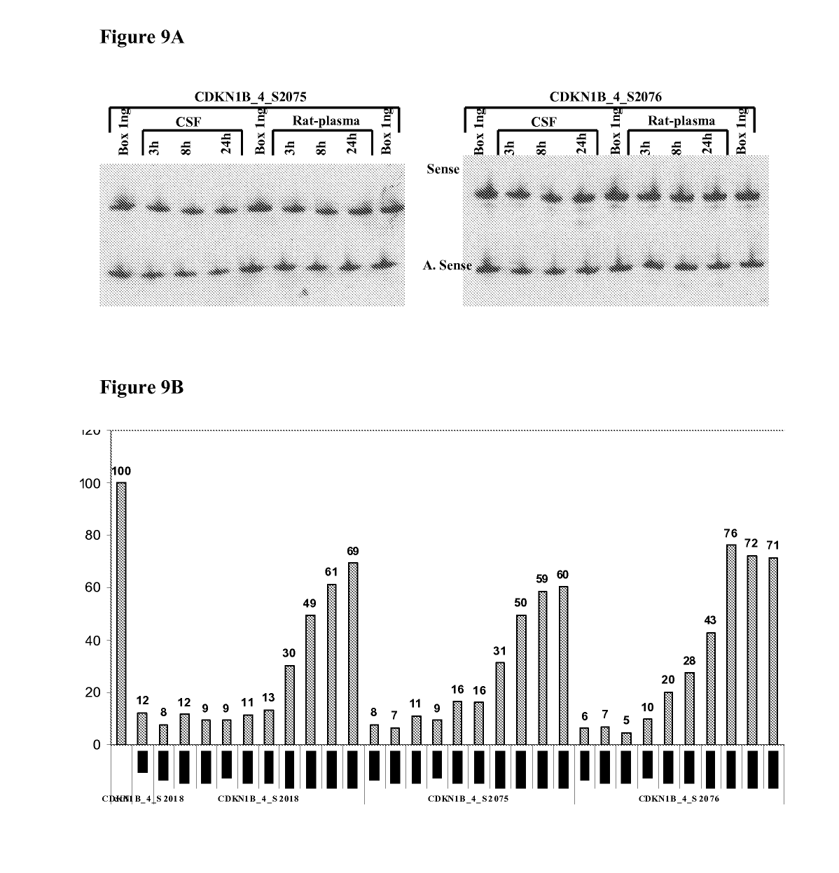

CDKN1B_{—}4_S2018, CDKN1B_{—}4_S2075 and CDKN1B_{—}4_S2076 are identical with respect to antisense strand and sense strand SEQ ID NOS (26895 and 26888), and with respect to antisense strand and sense strand modifications (antisense strand includes 2′OMe sugar modified ribonucleotides at positions 1, 13 and 17; a 2′5′ ribonucleotide in position 7 and a C3Pi-C3OH moiety covalently attached at the 3′ terminus; sense strand includes 2′OMe sugar modified ribonucleotides at positions 2, 6, 15 and 18 and a C3Pi moiety covalently attached at the 3′ terminus) and differ in that CDKN1B_{—}4_S2018 has no 5′ capping moiety on its sense strand while CDKN1B_{—}4_S2075 and CDKN1B_{—}4_S2076 have an inverted deoxyabasic capping moiety and a Amino C6 capping moiety, respectively, on its sense strand. Activity for all three is similar (Table G) but off target knock down potential for CDKN1B_{—}4_S2075 and CDKN1B_{—}4_S2076 is less than for CDKN1B_{—}4_S2018. Plasma stability of CDKN1B_{—}4CDKN1B_{—}4_S2075 and CDKN1B_{—}4_S2076 is shown in FIG. 9A. Knock-down activity is shown in FIG. 9B.

### Example 2

### Generation of Sequences for Active dsRNA Molecules to the Target Genes and Production of the siRNAs

Using proprietary algorithms and the known sequence of the mRNA of the target genes disclosed herein, the sequences of many potential dsRNA, i.e. siRNAs were generated. A key to the sequence listing is set forth hereinbelow:

SEQ ID NOS:23-26,912 set forth sense and antisense oligonucleotide sequences for generating dsRNA useful to down-regulate the expression of the following genes: HES1, HES5, HEY1, HEY2, ID1, ID2, ID3, CDKN1B or NOTCH1. Each sense and antisense oligonucleotide sequence is presented in 5′ to 3′ orientation.

Specifically, SEQ ID NOS:23-381 provide human 19 mer oligonucleotides; SEQ ID NOS:382-693 provide best 19-mer human-cross species oligonucleotides; SEQ ID NOS:694-1367 provide human 18 mer oligonucleotides; and SEQ ID NO:16-1495 provide best 18-mer human-cross species oligonucleotides useful in generating dsRNA to down-regulate HES1 expression; Table I includes certain preferred 19 mer oligonucleotides based on Structure A1, set forth in SEQ ID NOS: 26,667-26,690 and based on Structure A2, set forth in SEQ ID NOS:26,691-26,706 useful in generating dsRNA to down-regulate HES1 expression.

SEQ ID NOS:1496-1759 provide human 19 mer oligonucleotides; SEQ ID NOS:1760-2029 provide best 19-mer human-cross species oligonucleotides; SEQ ID NOS:2030-2575 provide human 18 mer oligonucleotides; and SEQ ID NOS:2576-2703 provide best 18-mer human-cross species oligonucleotides useful in generating dsRNA to down-regulate

HES5 expression; Table II includes certain preferred 19 mer oligonucleotides based on Structure A1, set forth in SEQ ID NOS: 26,707-26,724 and based on Structure A2, set forth in SEQ ID NOS:26,725-26,732 useful in generating dsRNA to down-regulate HES5 expression.

SEQ ID NOS:10534-11267 provide human 19 mer oligonucleotides; SEQ ID NOS:11268-11549 provide best 19-mer human-cross species oligonucleotides; SEQ ID NOS:11550-12903 provide human 18 mer oligonucleotides; and SEQ ID NOS:12904-13003 provide best 18-mer human-cross species oligonucleotides useful in generating dsRNA to down-regulate HEY1 expression; Table III includes certain preferred 19 mer oligonucleotides based on Structure A1, set forth in SEQ ID NOS: 26,733-26,760 and based on Structure A2, set forth in SEQ ID NOS: 26,761-26,778 useful in generating dsRNA to down-regulate HEY1 expression.

SEQ ID NOS:13004-14077 provide human 19 mer oligonucleotides; SEQ ID NOS:14078-14801 provide best 19-mer human-cross species oligonucleotides; SEQ ID NOS:14802-16389 provide best human 18 mer oligonucleotides; and SEQ ID NOS:16390-16621 provide best 18-mer human-cross species oligonucleotides useful in generating dsRNA to down-regulate HEY2 expression; Table IV includes certain preferred 19 mer oligonucleotides based on Structure A1, set forth in SEQ ID NOS: 26,779-26,784 and based on Structure A2, set forth in SEQ ID NOS:26,785-26,788 useful in generating dsRNA to down-regulate HEY2 expression.

SEQ ID NOS:2704-2941 provide human 19 mer oligonucleotides; SEQ ID NOS:2942-3025 provide best 19-mer human-cross species oligonucleotides; SEQ ID NOS:3026-3575 provide human 18 mer oligonucleotides; and SEQ ID NOS:3576-3633 provide best 18-mer human-cross species oligonucleotidesuseful in generating dsRNA to down-regulate ID1 expression; Table V includes certain preferred 19 mer oligonucleotides based on Structure A1, set forth in SEQ ID NOS:26,789-26,808 and based on Structure A2, set forth in SEQ ID NOS:26,809-26,816 useful in generating dsRNA to down-regulate ID1 expression.

SEQ ID NOS:3634-4107 provide human 19 mer oligonucleotides; SEQ ID NOS:4108-5053 provide best human-cross species oligonucleotides; SEQ ID NOS:5054-5751 provide human 18 mer oligonucleotides; and SEQ ID NOS:5752-6205 provide best 18-mer human-cross species oligonucleotides useful in generating dsRNA to down-regulate ID2 expression; Table VI includes certain preferred 19 mer oligonucleotides based on Structure A1, set forth in SEQ ID NOS:26,817-26,8240 and based on Structure A2, set forth in SEQ ID NOS:26,8251-26,832 useful in generating dsRNA to down-regulate ID2 expression.

SEQ ID NOS:6206-6531 provide human 19 mer oligonucleotides; SEQ ID NOS:6532-6671 provide best human-cross species oligonucleotides; SEQ ID NOS:6672-7353 provide human 18 mer oligonucleotides; and SEQ ID NOS:7354-7443 provide best 18-mer human-cross species oligonucleotides useful in generating dsRNA to down-regulate ID3 expression; Table VII includes certain preferred 19 mer oligonucleotides based on Structure A1, set forth in SEQ ID NOS:26,833-26,850 and based on Structure A2, set forth in SEQ ID NOS:26,851-26,866 useful in generating dsRNA to down-regulate ID3 expression.

SEQ ID NOS:7444-8185 provide human 19 mer oligonucleotides; SEQ ID NOS:8186-9007 provide best human-cross species oligonucleotides; SEQ ID NOS:9008-10233 provide human 18 mer oligonucleotides; and SEQ ID NOS:10234-10533 provide best 18-mer human-cross species oligonucleotides useful in generating dsRNA to down-regulate CDKN1B expression; Table VIII includes certain preferred 19 mer oligonucleotides based on Structure A1, set forth in SEQ ID NOS:26,867-26,886 and based on Structure A2, set forth in SEQ ID NOS:26,887-26,900 useful in generating dsRNA to down-regulate CDKN1B expression.

SEQ ID NOS:16622-18469 provide human 19 mer oligonucleotides; SEQ ID NOS:18470-18643 provide best human-cross species oligonucleotides; SEQ ID NOS:18644-26211 provide human 18 mer oligonucleotides; and SEQ ID NOS:26212-26666 provide best 18-mer human-cross species oligonucleotides useful in generating dsRNA to down-regulate NOTCH1 expression; Table IX includes certain preferred 19 mer oligonucleotides based on Structure A1, set forth in SEQ ID NOS:26,901-26,910 and based on Structure A2, set forth in SEQ ID NOS: 26,911-26,912 useful in generating dsRNA to down-regulate NOTCH1 expression.

The oligonucleotide sequences prioritized based on their score in the proprietary algorithm as the best predicted sequences for targeting the human gene expression.

“18+1” refers to a molecule that is 19 nucleotides in length and includes a mismatch to the mRNA target at position 1 of the antisense strand, according to Structure A2. In preferred embodiments the sense strand is fully complementary to the antisense strand. In some embodiments the sense strand is mismatched to the antisense strand in 1, 2, or 3 positions.

### Example 3

### On-Target and Off-Target Testing of Double Stranded RNA Molecules

The psiCHECK™ system enables evaluation of the guide strand (GS) (antisense) and the passenger strand (PS) (sense strand) to elicit targeted (on-target) and off-targeted effects, by monitoring the changes in expression levels of their target sequences. Four psiCHECK™-2-based (Promega) constructs were prepared for the evaluation of target activity and potential off-target activity of each test molecule GS and PS strands. In each of the constructs one copy or three copies of either the full target or the seed-target sequence, of test molecule PS or GS, was cloned into the multiple cloning site located downstream of the Renilla luciferase translational stop codon in the 3′-UTR region. The resulting vectors were termed:

GS-CM (guide strand, complete-match) vector containing one copy of the full target sequence (nucleotide sequence fully complementary to the whole 19-base sequence of the GS of the test molecule);

PS-CM (passenger strand, complete-match) vector containing one copy of the full target sequence (nucleotide sequence fully complementary to the whole 19-base sequence of the PS of the test molecule);

GS-SM (guide strand, seed-match) vector containing one copy or three copies of the seed region target sequence (sequence complementary to nucleotides 1-8 of the GS of the test molecule);

PS-SM (passenger strand, seed-match) vector containing one copy of the seed region target sequence (sequence complementary to nucleotides 1-8 of the PS of the test molecule).

### Nomenclature:

guide strand: strand of siRNA that enters the RISC complex and guides cleavage/silencing of the complementary RNA sequence

seed sequence:Nucleotides 2-8 from the 5′ end of the guide strand.

cm (complete match): DNA fragment fully complementary to the guide strand of siRNA. This DNA fragment is cloned in 3′UTR of a reporter gene and serves as a target for the straightforward RNA silencing.

sum (seed match): 19-mer DNA fragment with nucleotides ns 12-18 fully complementary to the ns 2-8 of the guide strand of siRNA. This DNA fragment is cloned in 3′UTR of a reporter gene and serves as a target for the “off-target” silencing.
X1: A single copy of cm or sum cloned in 3′UTR of a reporter gene.
X3 Three copies of cm or sum cloned in 3′UTR of a reporter gene, separated with 4 nucleotides one from another.

### Example 4

### The Effect of Target Gene dsRNA Treatment on Carboplatin-Induced Hair Cell Death in the Cochlea of Chinchilla

Eight Chinchillas are pre-treated by direct administration of HES1, HES5, HEY1, HEY2, ID1, ID2, ID3, CDKN1B, or NOTCH1 siRNA in saline (a compound as disclosed in Tables 2A-10D.) to the left ear of each animal. Saline is administered to the right ear of each animal as placebo. Two days following the administration of the siRNA, the animals are treated with carboplatin (75 mg/kg iP). After sacrifice of the chinchillas (two weeks post carboplatin treatment) the % of dead cells of inner hair cells (IRC) and outer hair cells (ONC) is calculated in the left ear (siRNA treated) and in the right ear (saline treated). Since the effect of the siRNA is similar across dose, the data is pooled from the 3 doses. As was previously shown, carboplatin preferentially damages the inner hair cells in the chinchilla at the 75 mg/kg dose while the outer hair cells remain intact. The dsRNA compounds provided herein reduce ototoxin-induced (e.g. carboplatin-induced) inner hair cells loss in the cochlea.

### Example 5

### The Effect of dsRNA Treatment on Acoustic-Induced Hair Cell Death in the Cochlea of Chinchilla

The activity of the dsRNA molecules of the present invention in an acoustic trauma model is studied in chinchilla. A group of 7 animals undergo acoustic trauma by exposing them to an octave band of noise centered at 4 kHz for 2.5 h at 105 dB. The left ear of the noise-exposed chinchillas is pre-treated (48 h before the acoustic trauma) with about 30 μg of siRNA in ˜10 μL of saline; the right ear is pre-treated with vehicle (saline). The compound action potential (CAP) is a convenient and reliable electrophysiological method for measuring the neural activity transmitted from the cochlea. The CAP is recorded by placing an electrode near the base of the cochlea in order to detect the local field potential that is generated when a sound stimulus, such as click or tone burst, is abruptly turned on. The functional status of each ear is assessed at about 2.5 weeks after the acoustic trauma. Specifically, the mean threshold of the compound action potential recorded from the round window is determined 2.5 weeks after the acoustic trauma in order to determine if the thresholds in the dsRNA-treated ear were lower (better) than the untreated (saline) ear. In addition, the amount of inner and outer hair cell loss is determined in the siRNA-treated and the control ear. The results indicate that dsRNAs provided herein, are capable of reducing acoustic trauma-induced ONC loss in the cochlea.

### Example 6

### The Effect of dsRNA Treatment on Cisplatin-Induced Hair Cell Death in the Cochlea of Rats

Male Wistar Rats are tested for basal auditory brainstem response (ABR) thresholds for signals of clicks, 8, 16 and 32 kHz prior to cisplatin treatment. Following the basal auditory brainstem response testing, cisplatin is administered as an intraperitoneal infusion of 12 mg/kg over 30 minutes. Treated ears receive either 1 ug/4 microliter of dsRNA disclosed herein in PBS (applied directly to the round window membrane). Control ears are treated either with non-related GFP dsRNA or PBS. The dsRNA molecules are administered between 3-5 days prior to cisplatin administration in order to permit protective effect on the cochlea.

The auditory brainstem response (ABR) testing is repeated 3 days after cisplatin administration. The auditory brainstem response thresholds are compared between pretreatment and post treatment and the shift in thresholds is measured. Higher shift in thresholds following cisplatin treatment is indicative for more severe hair cells loss in the cochlea. After the repeat of auditory brainstem response testing, animals are sacrificed and cochleae are removed and processed for scanning electron microscopy (SEM) to quantify outer hair cell (ONC) loss in the hook region (high frequency region). The % outer hair cell loss is calculated by dividing the number of missing or severely damaged cells by the total number of outer hair cells in the field of the photograph. The results indicate that dsRNAs compounds disclosed herein provide a protective effect to the cochlea when administered prior to ototoxin (e.g. cisplatin) administration.

### Example 7

### Additional Hearing Loss Models

### a) Hearing Regeneration (Plasticity) Model in Guinea-Pig

Deafening is induced by systemically treating albino guinea pigs with a single is injection of kanamycin (450-500 mg/kg) followed by a single iv (jugular) injection of ethacrynic acid (EA). This pharmacological deafening eliminates bilaterally all hair cells approximately after 1-2 days and leaves the supporting cells differentiated. Therapeutic nucleic acids are applied to the middle ear by transtympanic injection (TT) or into the external auditory canal or eardrum by ear drops (ErD).

dsRNAs which target HES1, HES5, HEY1, HEY2, ID1, ID2, ID3, CDKN1B, or NOTCH1 are applied as described above.

The efficacy of the dsRNAs are examined as follows:

1) Cochleae/s are morphologically analyzed as whole-mounts stained for myosin VIIa (hair cell marker) and phalloidin.

2) BrdU incorporation is measured as an indicator of proliferation rate of hair cells.

### B) Noise Induced Acute Hearing Loss Model in Guinea Pig

Noise can cause hearing damage with temporary or permanent sensorineural hearing loss (SNHL) and tinnitus. SNHL and tinnitus can occur singular or in combination. In humans, noise induced hearing loss (NIHL) is demonstrated by a threshold shift in the pure tone audiogram, in recruitment, in pathological results of supra-threshold hearing tests and in amplitude decline of oto-acoustic emissions. Hearing damage is induced by exposure to continuous noise or impulsive noise. In addition the possibility of impulse noise traumata or explosion trauma should be taken into consideration. Exposure to impulse noise can result in a more severe lesion of the inner ear than exposure to continuous noise. Important criteria for the development of noise damage are sound pressure level (SPL), level increase velocity, exposure time, as well as individual susceptibility (“the vulnerable inner ear”). Noise exposure usually leads to an elevation of threshold which may be later resolved in part, such that the temporary component is called “temporary threshold shift” (TTS). If there isn't complete restitution in the recovery phase after TTS, this may result in permanent inner ear damage (permanent threshold shift=PTS). Very high sound intensity may lead to immediate cellular death and mechanical rupture of structures in the inner ear and PTS.

In this model, a bilateral lesion is induced with noise exposure; Guinea pigs are exposed to 117 dB SPL broadband noises for 6 hours.

In a pilot study according to this model, dsRNAs which target HES1, HES5, HEY1, HEY2, ID1, ID2, ID3, CDKN1B, or NOTCH1 are employed in this model with similar results.

### Example 8

### dsRNA in Supporting Cells of a Deaf Animal

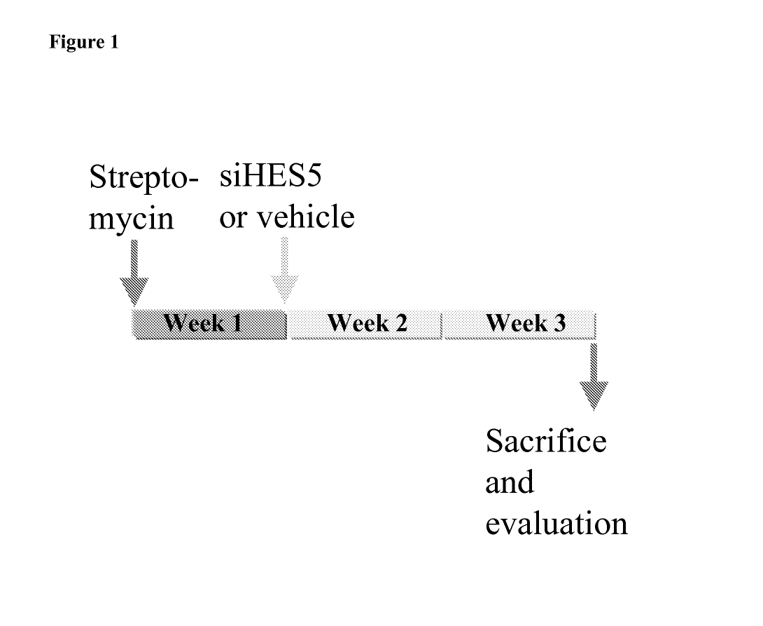

The experimental design was essentially as depicted in FIG. 1. The aminoglycoside, streptomycin was used to induce inner ear hair loss in mice. Streptomycin (200 mg/ml) was injected into the posterior semi-circular canal (PSC) and seven days later dsRNA to HES5 (2 ug/ug) was injected into PSC. Inner ear hair cell regeneration was evaluated at two weeks after dsRNA injection.

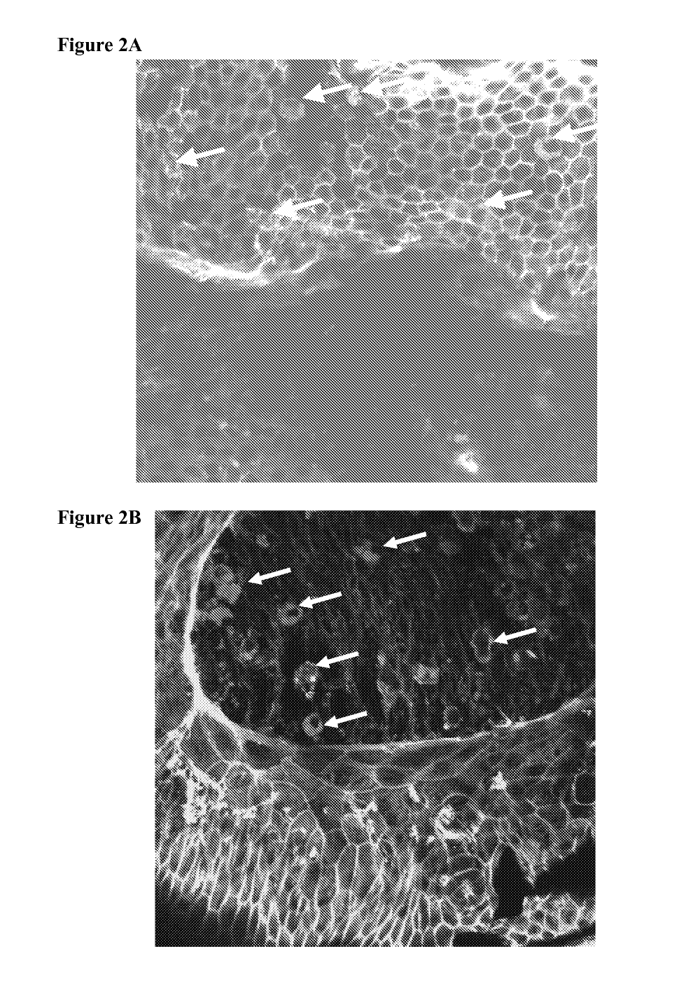

Preliminary experiments were performed to show that dsRNA can be delivered to the target cells in the PSC by applying Cy3 labeled dsRNA to the PSC. dsRNA is delivered to normal vestibular sensory epithelium upon local injection into the PSC. (figure not shown). More importantly, dsRNA is delivered to AG-damaged vestibular sensory epithelium upon local injection into PSC (FIGS. 2A-2B).

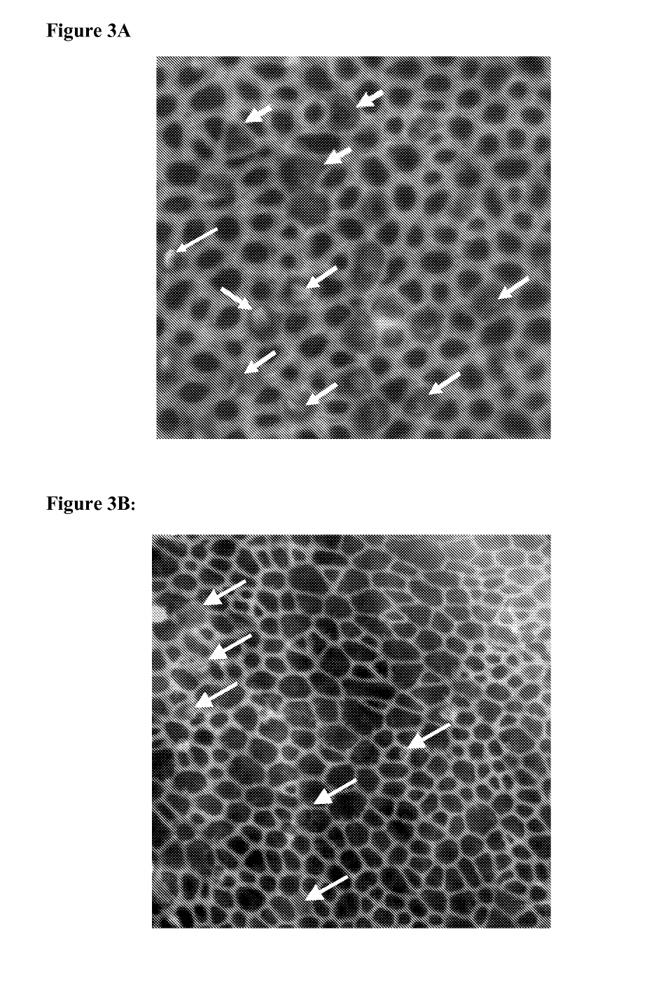

FIGS. 3A-3B show that treatment with dsRNA to HES5 increases the amount of hair cells in AG-damaged vestibular epithelia.

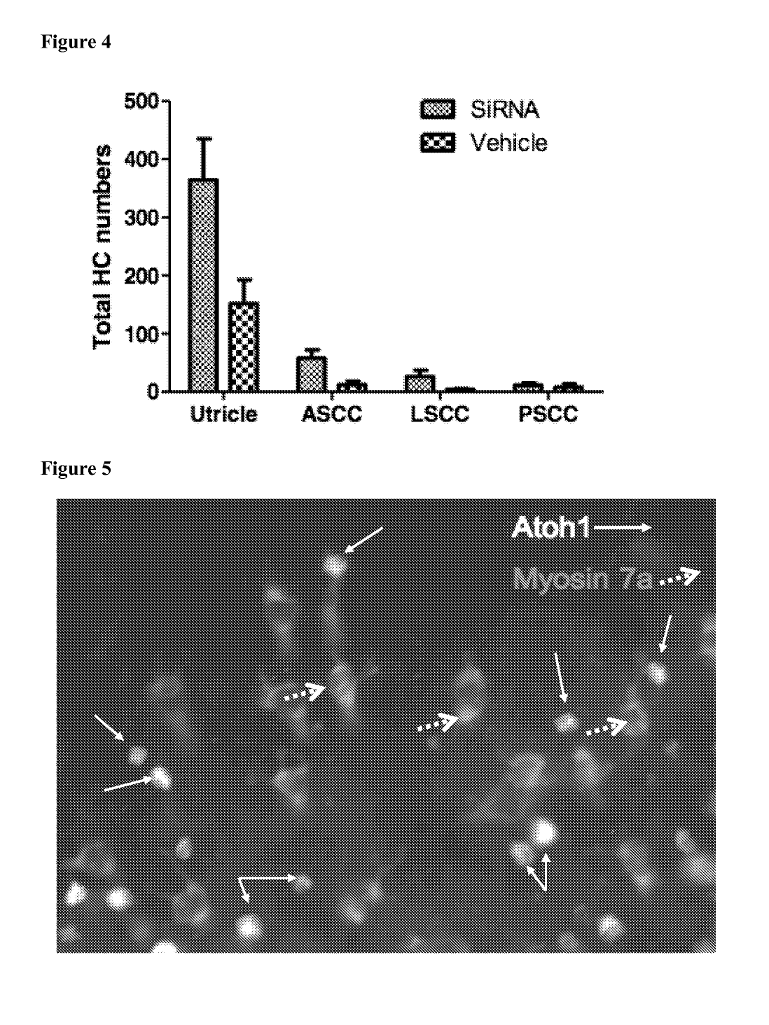

FIG. 4 shows that treatment with dsRNA to HES5 facilitates hair cell regeneration. The y-axis shows hair cell counts. The columns are labeled as follows: ASCC—anterior semicircular canal, LSCC—lateral semicircular canal, PSCC—posterior semicircular canal.

Treatment with dsRNA to HES5 increases the amount of vestibular hair cells (Atoh1-positive) as identified using double anti-Atoh1 and anti-Myosin VIIa staining (FIG. 5).

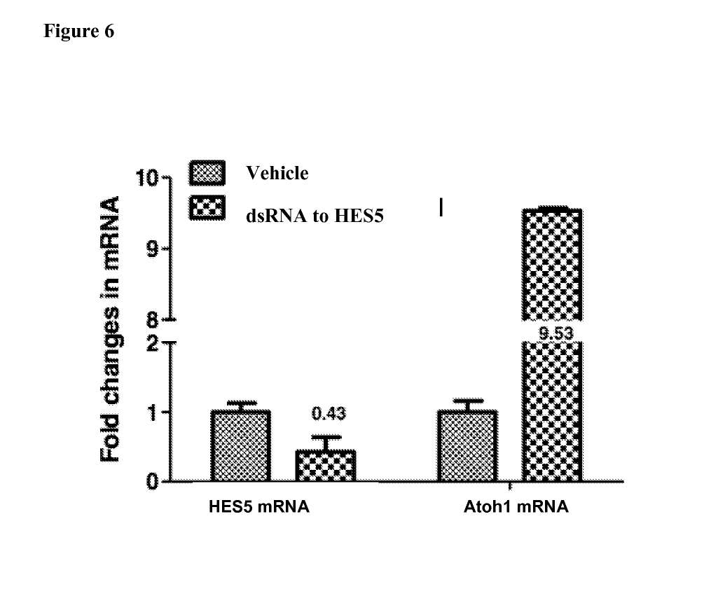

FIG. 6 shows that treatment with dsRNA to HES5 down-regulates HES5 and up-regulates Atoh1 expression.

### CONCLUSIONS

1. siRNA given through posterior semicircular canalostomy can affect vestibular sensory epithelium in both normal and AG damaged mice vestibule.

2. Vestibular hair cell regeneration at 3 weeks after AG damage was facilitated by Hes5 siRNA administration.

The sequence of the Hes5 dsRNA used was as follows:

sense strand:

(SEQ ID NO: 26728)

GGGUUCUAUGAUAUUUGUA; structure:
rG;mG;rG;mU;rU;mC;rU;mA;rU;mG;rA;mU;rA;mU;rU;mU;
rG;mU;rA
Antisense strand:

(SEQ ID NO: 26732)

UACAAAUAUCAUAGAACCC; structure:
mU;rA;mC;rA;mA;rA;mU;rA;mU;rC;mA;rU;mA;rG;mA;rA;
mC;rC;mC

3. Atoh1 positive cell numbers at 2 weeks after AG damage are higher in Hes5 dsRNA treated mice.

4. Expression of Hes5 mRNA was further down regulated in the siRNA treated group.

5. Expression of Atoh1 mRNA was further up-regulated in the siRNA treated group.

### Example 9

### The Effect of Target Gene siRNA Treatment on Noise-Induced Death of Otic Sensory Cells of the Inner Ear

### Model System:

Exposure of guinea pigs to one-octave-band noise centered at 6 kHz, at 130 dB SPL for 2 hours (Futon et al, NeuroReport 19:277-281, 2008)

### Experimental Groups

Adult Hartley albino guinea pigs (age 3 months), with normal Preyer's reflex, are exposed to noise and randomized to the following groups: noise control group without treatment (n=6), noise control animals with vehicle (n=6), animals treated with dsRNA compound provided herein which down-regulates expression of the HES1 gene at a dose of 1 μg (n=6); animals treated with dsRNA compound provided herein which down-regulates expression of HES1 gene at a dose of 5 μg (n=6); animals treated with dsRNA compound provided herein which down-regulates expression of HES1 gene at a dose of 10 μg (n=6); animals treated with dsRNA compound provided herein which down-regulates expression of HES1 gene at a dose of 50 μg (n=6); 4 groups of noise control animals with control dsRNA compound which down-regulates expression of EGFP gene (each n=6), at a dose of 1 μg, 5 μg, 10 μg and 50 μg respectively.

Treatment is performed 1 h before noise exposure and once daily for 3 days thereafter.

The following exemplary vehicles are used in this experiment: PBS, artificial perilymph solution.

The dsRNA test compound and the dsRNA control compound are formulated for administration in the vehicle of the experiment.

Vehicle, dsRNA test compound or dsRNA control compound, is injected intraperitoneally or by bolus injection. All animals are sacrificed after functional evaluation with a lethal dose of anaesthetic: three animals for each group at day 1 for immunolabeling and the remaining animals at day 21, of which three are further processed for scanning electron microscopy (SEM).

### Noise Exposure

Acoustic trauma is induced by a continuous pure tone of 6 kHz generated by a waveform generator (for example: Generator LAG-120B, Leader Electronics Corp, Yokohama, Japan), and amplified by an audio amplifier (for example: A-207R, Pioneer Electronics, Long Beach, Calif., USA). All animals, under anaesthetic, are exposed for 40 min to a 6 kHz, 120 db SPL (Sound Pressure Level) sound presented in an open field (for example: dome tweeter TW340×0, Audax, Chateau de Loir, France).

### Electrophysiological Measurements of Auditory Function

Auditory brainstem responses (ARB) are measured before noise exposure and 1 h, 3 days, 7 days and 21 days after noise exposure. Animals are mildly anaesthetized and placed in a soundproof room. Three electrodes are subcutaneously inserted into the right mastoid (active), vertex (reference) and left mastoid (ground). A computer-controlled data acquisition system, for example TDT System 3 (Tucker-Davis Technologies, Alachu, Fla., USA) data acquisition system with real-time digital signal processing is used to record the ABR and to generate the auditory stimulus. Tone bursts of pure tones ranging from 2 to 24 kHz (rise/fall time, 1 ms; total duration, 10 ms; repetition rate, 20/s) is presented monaurally in an open field. Responses are filtered (0.3-3 kHz), digitized and averaged across 500 discrete samples at each frequency-level combination.

### Morphological Studies: Scanning Electron Microscopy

SEM analysis is performed, e.g. as described in Sergi B, et al. Protective properties of idebenone in noise-induced hearing loss in the guinea pig. NeuroReport 2006; 17:857-861. Briefly, the cochlea (n=3) of three animals for each group is perfused with 2.5% glutaraldehyde in 0.1 M phosphate buffer and post-fixed overnight and then incubated for 2 h in 2% osmium tetroxide cacodylate buffer. After micro-dissection, the cochlea is dehydrated with increasing concentrations of ethanol from 30 to 100% and dried in the critical point and finally coated with gold. Each specimen is viewed and photographed by means of, e.g. a Zeiss Supra 50 Field Emission SEM apparatus (Carl Zeiss Inc., Gottingen, Germany). Quantitative EM observations of the surface morphology of the organ of Corti are performed by determining the number of hair cells in 20 segments (1 mm length of basilar membrane each). A hair cell is counted as missing if the stereociliary bundle is absent or the stereocillia of the bunch are completely fused. Results of hair cell counts are expressed as the percentage of remaining hair cells in each row of inner hair cells and outer hair cells over the entire length of cochlea.

Terminal Deoxynucleotidyl Transferase Mediated dUTP Nick End Labeling Assay

The cochlea (n=3) of three animals for each group are stained by using TUNEL (terminal deoxynucleotidyl transferase-mediated dUTP nick end labeling) assay (for example, Molecular Probes, Inc., Carslbad, Calif., USA) as described in B Sergi et al. Protective properties of idebenone in noise-induced hearing loss in the guinea pig. NeuroReport (2006) 17:857-861. Briefly, the cochlea are fixed with 10% formaldehyde in 0.1 M phosphate-buffered saline (PBS), pH 7.3. After micro-dissection, surface preparations of the organ of Corti are incubated in ice-cold 70% (v/v) ethanol overnight and then in freshly prepared DNA labeling solution containing 10 μl of reaction buffer, 0.75 μl of TdT enzyme, 8.0 μl of BrdUTP and 31.25 μl of dH₂O for 16 h at room temperature. The tissues are then stained with Alexa Fluor 488 dye-labelled anti BrdU antibody—contained in the TUNEL assay kit (e.g., Molecular Probes Inc., Carlsbad, Calif., USA) (5 μl of antibody plus 95 μl of the Corti are double stained with propidium iodide (5 μg/ml in 10 mM PBS) for 20 min at room temperature. After rinsing in PBS, the organs of Corti are mounted on slides containing an anti-fade medium (for example, Prolong Gold, Molecular Probes, Inc.). Specimens are observed using confocal laser scanning microscopy (e.g., Leica TCS—SP2, Leica Inc., Wetzlar, Germany).

### Results

The results obtained in this model indicate that dsRNAs provided herein:
(a) attenuated noise-induced threshold shift;
(b) decreased noise-induced outer hair cell loss; provided protection against noise-induced hearing loss (NIHL).

This model is useful for testing the efficacy of dsRNA molecules that have the potential to down-regulate, for example HEST, HES5, HEY1, HEY2, ID1, ID2, ID3, CDKN1B, or NOTCH1 genes

Although the above examples have illustrated particular ways of carrying out embodiments of the invention, in practice persons skilled in the art will appreciate alternative ways of carrying out embodiments of the invention, which are not shown explicitly herein. It should be understood that the present disclosure is to be considered as an exemplification of the principles of this invention and is not intended to limit the invention to the embodiments illustrated.

Those skilled in the art will recognize, or be able to ascertain using no more than routine experimentation, equivalents of the specific embodiments of the invention described herein. Such equivalents are intended to be encompassed by the following claims.

1-65. (canceled)

66. A method of treating a hearing disorder/hearing loss in a subject, wherein the etiology or progression of the hearing disorder/hearing loss is associated with expression of a HES1 gene, comprising administering to the subject a therapeutically effective amount of an inhibitor of the HES1 gene, thereby treating the hearing disorder/hearing loss in the subject.

67. A method of treating a disease or disorder of the vestibular system in a subject, wherein the etiology or progression of the disease or disorder is associated with expression of a HES5 gene, the method comprising administering to the subject a therapeutically effective amount of an inhibitor of the HES5 gene, thereby treating the balance impairment in the subject.

68. The method of claim 66, wherein the inhibitor of the HES1 gene, promotes regeneration of hair cells in a cochlea of the subject.

69. The method of claim 67, wherein the inhibitor of the HES5 gene, promotes regeneration of hair cells in a vestibular epithelia of the subject.

70. A double-stranded nucleic acid molecule having a structure (A2) set forth below:

| | | |
|---|---|---|
| (A2) | 5′ N¹-(N)x-Z 3′ | (antisense strand) |
| | 3′ Z′-N²-(N′)y-z″ 5′ | (sense strand) |

wherein each of N², N and N′ is an unmodified ribonucleotide, a modified ribonucleotide, or an unconventional moiety;
wherein each of (N)x and (N′)y is an oligonucleotide in which each consecutive N or N′ is joined to the adjacent N or N′ by a covalent bond;
wherein each of x and y is independently an integer between 17 and 39;
wherein the sequence of (N′)y has complementarity to the sequence of (N)x and (N)x has complementarity to a consecutive sequence in a target mRNA set forth in SEQ ID NO: 1-11;
wherein N¹ is covalently bound to (N)x and is mismatched to the target mRNA or is a complementary deoxyribonucleotide moiety to the target mRNA;
wherein N¹ is a moiety selected from the group consisting of a natural or a modified uridine, deoxyribouridine, ribothymidine, deoxyribothymidine, adenosine or deoxyadenosine;
wherein z″ may be present or absent, but if present is a capping moiety covalently attached at the 5′ terminus of N²—(N′)y; and
wherein each of Z and Z′ is independently present or absent, but if present is independently 1-5 consecutive nucleotides, 1-5 consecutive non-nucleotide moieties or a combination thereof covalently attached at the 3′ terminus of the strand in which it is present.

71. The double-stranded nucleic acid molecule of claim 70, wherein x=y=18.

72. The double-stranded nucleic acid molecule of claim 71, wherein (N)x has complementarity to a consecutive sequence in a target mRNA set forth in SEQ ID NO:1 (HES1).

73. The double-stranded nucleic acid molecule of claim 72, wherein (N)x is SEQ ID NO:1162 and (N′)y is SEQ ID NO:825.

74. The double-stranded nucleic acid molecule of claim 72, comprising a nucleic acid described as HES1_{—}14 (SEQ ID NOS:26681 and 26669) or HES1_{—}36 (SEQ ID NOS:26690 and 26678).

75. The double-stranded nucleic acid molecule of claim 71, wherein (N)x has complementarity to a consecutive sequence in a target mRNA set forth in SEQ ID NO:2 (HES5).

76. The double-stranded nucleic acid molecule of claim 71, wherein (N)x has complementarity to a consecutive sequence in a target mRNA set forth in SEQ ID NO:10 (HEY2).

77. The double-stranded nucleic acid molecule of claim 76, wherein (N)x is SEQ ID NO:15649 and (N′)y is SEQ ID NO:14855.

78. The double-stranded nucleic acid molecule of claim 76, comprising a nucleic acid described as HEY2_{—}2 (SEQ ID NOS:26783 and 26669).

79. A composition comprising the double-stranded nucleic acid molecule of claim 70; and a pharmaceutically acceptable carrier.

80. A double-stranded nucleic acid molecule having the structure (A1):

| | | |
|---|---|---|
| (A1) | 5′ (N)x-Z 3′ | (antisense strand) |
| | 3′ Z′-(N′)y-z″ 5′ | (sense strand) |

wherein each N and N′ is an unmodified ribonucleotide, a modified ribonucleotide, or an unconventional moiety;
wherein each of (N)x and (N′)y is an oligonucleotide in which each consecutive N or N′ is joined to the next N or N′ by a covalent bond;
wherein each of Z and Z′ is independently present or absent, but if present independently comprises 1-5 consecutive nucleotides, 1-5 consecutive non-nucleotide moieties or a combination thereof covalently attached at the 3′ terminus of the strand in which it is present;
wherein z″ may be present or absent, but if present is a capping moiety covalently attached at the 5′ terminus of (N′)y;
wherein each of x and y is independently an integer from 18 to 40; and
wherein the sequence of (N′)y has complementarity to the sequence of (N)x and (N)x has complementarity to a consecutive sequence in a target mRNA set forth in SEQ ID NO: 1-11.

81. The double-stranded nucleic acid molecule of claim 80, wherein x=y=19.

82. The double-stranded nucleic acid molecule of claim 81, wherein (N)x has complementarity to a consecutive sequence in a target mRNA set forth in SEQ ID NO:1 (HES1), and wherein (N)x and (N′)y are sequence pairs set forth in any one of SEQ ID NOS:23-693 and 26691-26706.

83. The double-stranded nucleic acid molecule of claim 81, wherein (N)x has complementarity to a consecutive sequence in a target mRNA set forth in SEQ ID NO:2 (HES5), and wherein (N)x and (N′)y are sequence pairs set forth in any one of SEQ ID NOS:1496-2029 and 26725-26732 (HES5).

84. The double-stranded nucleic acid molecule of claim 81, wherein (N)x has complementarity to a consecutive sequence in a target mRNA set forth in SEQ ID NO:10 (HEY2), and wherein (N)x and (N′)y are sequence pairs set forth in any one of SEQ ID NOS:13004-14801 and 26785-26788.

85. The double-stranded nucleic acid molecule of claim 83, comprising a nucleic acid described as HES5_{—}8 (SEQ ID NOS:26732 and 26728).

86. A composition comprising the double-stranded nucleic acid molecule of claim 80; and a pharmaceutically acceptable carrier.
